(19) United States
(12) Patent Application Publication
(10) Pub. No.: US 2025/0340622 A1
(43) Pub. Date: Nov. 6, 2025

(54) PREVENTION OF RESPIRATORY SYNCYTIAL VIRUS LOWER RESPIRATORY TRACT INFECTION WITH NIRSEVIMAB

(71) Applicants: MEDIMMUNE LIMITED, Cambridge (GB); SANOFI PASTEUR INC., Swiftwater, PA (US)

(72) Inventors: Tonya Luana VILLAFANA, Wilmington, DE (US); Amanda LEACH, Wilmington, DE (US); Mark Thomas ESSER, Gaithersburg, MD (US); Deidre Elaine WILKINS, Gaithersburg, MD (US); Ulrika WÄHLBY HAMRÉN, Södertälje (SE)

(21) Appl. No.: 18/691,361

(22) PCT Filed: Sep. 12, 2022
(86) PCT No.: PCT/US2022/076311

§ 371 Date: Mar. 12, 2024

Related U.S. Application Data
(60) Provisional application No. 63/261,117, filed on Sep. 13, 2021.
(60) Provisional application No. 63/266,594, filed on Jan. 10, 2022.
(60) Provisional application No. 63/363,633, filed on Apr. 26, 2022.
(60) Provisional application No. 63/364,554, filed on May 11, 2022.

(51) Int. Cl. C07K 16/10; A61K 39/00; A61P 31/14

(52) U.S. Cl. CPC C07K 16/1027; A61P 31/14; A61K 2039/505; A61K 2039/54; A61K 2039/545

(57) ABSTRACT

The present disclosure provides methods of preventing, reducing the risk, or mitigating RSV lower respiratory tract infection by administering nirsevimab.

This application is a U.S. national stage entry under 35 U.S.C. § 371 of International Application No. PCT/US2022/076311, filed Sep. 12, 2022, which designates the U.S. and claims the benefit of U.S. Provisional Application Ser. No. 63/364,554, filed May 11, 2022; 63/363,633, filed Apr. 26, 2022; 63/266,594, filed Jan. 10, 2022; and 63/261,117, filed Sep. 13, 2021, each of which is incorporated by reference herein.

This application relates to preventing infection or other disease associated with Respiratory Syncytial Virus (RSV).

### SEQUENCE LISTING

This application contains a Sequence Listing titled 15826_0008-00304_SL.xml, which was created Oct. 6, 2022, and is 13,084 bytes in size. The Sequence Listing has been filed electronically in XML format and is hereby incorporated by reference in its entirety.

### BACKGROUND OF THE INVENTION

Respiratory syncytial virus (RSV) is a common cold virus belonging to the family of paramyxovirus. RSV is virulent and easily transmissible. It is the most common cause of lower respiratory tract infections (LRTI) among infants and young children, resulting in annual epidemics worldwide. All children are at risk for severe RSV LRTI. Ninety percent of children are infected with RSV in the first 2 years of life; and up to 40% of those will have LRTI with the initial episode. RSV LRTI, characterized predominantly as bronchiolitis or pneumonia, represents a serious illness with acute and even long-term consequences to the developing lungs in these young children (Blanken et al., N Engl J Med. (2013) 368 (19): 1791-9).

Severe LRTI episodes often lead to hospitalization. Major risk factors for hospitalization due to RSV infections are premature birth, chronic lung disease (CLD), congenital heart disease, compromised immunity, and age younger than 6 weeks in otherwise healthy children. However, a large percentage of the healthcare burden from RSV infections occurs outside the hospital as office visits and emergency department visits, especially in healthy infants.

Prevention of RSV illnesses in all infants is a major public health priority. Yet, despite more than 50 years of attempted vaccine development, there are no licensed vaccines. The only currently approved prophylaxis for RSV is palivizumab (Synagis®), a humanized monoclonal antibody targeting the fusion (F) protein of RSV. Palivizumab is only indicated for use in high-risk children: preterm infants 35 weeks or less gestational age (GA), children with CLD of prematurity, and children with hemodynamically significant congenital heart disease (CHD). In addition, further restrictions have been implemented by local or national recommending bodies on the use of palivizumab. For example, in the U.S., as per the American Academy of Pediatrics (AAP) guidelines, palivizumab is not recommended for healthy preterm infants 29 weeks or more GA.

There is a serious unmet medical need for protection against RSV in healthy preterm and term infants. Phase 3 data are available from a study evaluating maternal immunization using an RSV fusion (F) protein nanoparticle. However, that study failed to meet the primary endpoint with sufficient precision; the reported efficacy against medically significant RSV LRTI was 39.4% (97.52% CI, −1.0 to 63.7) (Madhi et al., N Engl J Med. (2020) 383:426-39). As there is no approved RSV prophylaxis for the broader population of healthy infants and no treatment for RSV, the current management for these patients when they acquire serious RSV illness is merely supportive care. Thus, there remains an urgent need for passive immunization of all infants to prevent or mitigate against RSV infections. Another consideration in preventing or mitigating against RSV infections in infants is the duration of RSV seasons. For example, an RSV season may last five months or longer, such as six, seven, eight, nine, or ten months. Palivizumab must be dosed monthly throughout the RSV season, which may limit accessibility. See IMpact-RSV Study Group. “Palivizumab, a humanized respiratory syncytial virus monoclonal antibody, reduces hospitalization from respiratory syncytial virus infection in high-risk infants.” Pediatrics 102.3 (1998): 531-537. Thus, there also remains an urgent need for passive immunization that is effective to prevent or mitigate against RSV infections in infants with a single dose. Similarly, there is a need for effective passive immunization with a single dose that can be administered outside the RSV season.

### SUMMARY OF THE INVENTION

The present disclosure provides, inter alia, a method of preventing RSV lower respiratory tract infection in an infant or pediatric subject in need thereof. In some embodiments, the method comprises administering to the subject a single dose of nirsevimab before the beginning of the RSV season, wherein the single dose is effective to prevent RSV LRTI for more than five months. In some embodiments, the method comprises administering to the subject a single dose of nirsevimab following the end of the last RSV season, wherein the single dose is effective to prevent RSV LRTI throughout the next RSV season.

In some embodiments, the method comprises administering to the subject a single dose of nirsevimab, wherein the administration occurs not more than once per RSV season. In some embodiments, the method comprises administering to the subject a single dose of nirsevimab, wherein the administration occurs not more than once per year. In some embodiments, the method comprises administering to the infant or pediatric subject a single dose of nirsevimab at a timepoint that is outside the RSV season, wherein the administration occurs not more than once per year. In some embodiments, the single dose of nirsevimab is administered intramuscularly.

In some embodiments, the method comprises administering to the infant or pediatric subject a single dose of nirsevimab per RSV season, wherein the administration is before the start of the season, e.g., one, two, three, or more months before. In some embodiments, the method comprises administering to the infant or pediatric subject a single dose of nirsevimab per RSV season that is sufficient to provide protection for more than five months, e.g., for at least six, seven, eight, nine, or ten months. In some embodiments, the single dose of nirsevimab is sufficient to provide protection for at least eleven or twelve months.

In some embodiments, the method comprises administering to the infant or pediatric subject a single dose of nirsevimab per RSV season at the beginning of the RSV season or during the RSV season, wherein the RSV season is longer than about five months, optionally wherein the RSV season is about six, about seven, about eight, about nine, or about ten months long. In some embodiments, the single dose of nirsevimab provides protection against RSV infection and/or against RSV disease for more than five months, preferably for at least six, seven, eight, nine, ten, eleven, or twelve months. In some embodiments, the single dose of nirsevimab is administered intramuscularly.

In some embodiments, the method comprises preventing very severe RSV infection in an infant or pediatric subject in need thereof. In some embodiments, the method comprises administering to the subject a single dose of nirsevimab, wherein the single dose is effective to prevent very severe RSV infection for more than five months.

In some embodiments, the method comprises preventing all-cause lower respiratory tract infection (LRTI) or all-cause LRTI hospitalization in an infant or pediatric subject in need thereof. In some embodiments, the method comprises administering to the subject a single dose of nirsevimab, wherein the single dose is effective to prevent all-cause LRTI or all-cause hospitalization for more than five months.

In some embodiments, the method comprises preventing RSV LRTI in a subject at high risk of developing an RSV infection (e.g., at high risk of developing RSV LRTI). In some embodiments, the method comprises administering to the subject a first dose of nirsevimab before or during the subject's first RSV season and a second dose of nirsevimab before or during the subject's second RSV season. In some embodiments, the amount of nirsevimab in the first dose is 50 mg if the subject weighs <5 kg at the time of administration and 100 mg if the subject weighs ≥5 kg at the time of administration. In some embodiments, the amount of nirsevimab in the second dose is 200 mg.

In some embodiments, the method comprises preventing RSV LRTI in a subject undergoing a heart surgery. In some embodiments, the method comprises administering to the subject a first dose of nirsevimab before the subject's first RSV season, wherein the amount of nirsevimab in the first dose is 50 mg if the subject weighs <5 kg at the time of administration of the first dose, or the amount of nirsevimab in the first dose is 100 mg if the subject weighs ≥5 kg at the time of administration of the first dose; and administering to the subject a second dose of nirsevimab after the heart surgery, wherein the amount of nirsevimab in the second dose is 50 mg if the subject weighs <5 kg at the time of administration of the second dose and the second dose is administered within 90 days of administration of the first dose, or the amount of nirsevimab in the second dose is 100 mg if the subject weighs ≥5 kg at the time of administration of the second dose and the second dose is administered within 90 days of administration of the first dose, or the amount of nirsevimab in the second dose is 50 mg if the second dose is administered more than 90 days after administration of the first dose. In some embodiments, the second dose is administered as soon as the subject is stable after the surgery. In some embodiments, the heart surgery is a cardiac surgery with cardiopulmonary bypass.

In some embodiments, the method comprises preventing RSV LRTI in a subject undergoing a heart surgery. In some embodiments, the method comprises administering to the subject a first dose of nirsevimab before the subject's second RSV season, wherein the amount of nirsevimab in the first dose is 200 mg; and administering to the subject a second dose of nirsevimab after the heart surgery, wherein the amount of nirsevimab in the second dose is 200 mg if the second dose is administered within 90 days of administration of the first dose, or the amount of nirsevimab in the second dose is 100 mg if the second dose is administered more than 90 days after administration of the first dose. In some embodiments, the second dose is administered as soon as the subject is stable after the surgery. In some embodiments, the heart surgery is a cardiac surgery with cardiopulmonary bypass.

In some embodiments, a dose of nirsevimab is administered at birth.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

The contents of references incorporated in their entirety herein are incorporated to the extent that they are not inconsistent with the present disclosure. In the event of an inconsistency, the present disclosure controls.

### BRIEF DESCRIPTION OF THE DRAWINGS

FIG. 1 is a diagram showing the design for the study described in Example 2. Blood samples for pharmacokinetics analyses were collected at screening or Day 1 pre-dose, on Days 31, 151, and 361, and from participants hospitalized for a respiratory infection through Day 361. Safety assessments were performed through Day 361. a: Dose levels were stratified by body weight at the time of dosing. In the nirsevimab group, participants received 50 mg (0.5 mL) nirsevimab if <5 kg or 100 mg (1.0 mL) nirsevimab if ≥5 kg. Participants in the placebo group received a corresponding volume of normal saline, i.e., 0.5 mL if <5 kg or 1.0 mL if ≥5 kg. b: In Japan, Day 15 visit was replaced by visit on Day 8 for blood sample collection (laboratory parameters). Blood samples were also collected at visits on Days 31 and 151 in Japan. In Europe, the Day 31 assessment for RSV serology was removed to limit the amount of blood drawn. Blood sample for pharmacokinetics analysis collected on Day 15 instead of Day 31. IM, intramuscular; LRTI, lower respiratory tract infection.

FIG. 2 is a graph showing the time to first medically attended RSV-associated LRTI in the intent-to-treat (ITT) population. A Kaplan-Meier curve from a time-to-event analysis shows an estimate of the proportion of participants who were free from a medically attended RSV-associated LRTI. The hazard ratio and corresponding 95% CIs were obtained from a stratified proportional-hazard model. Tick marks indicate censored data.

FIG. 3 is a graph showing the time to first medically attended RSV-associated LRTI in the ITT population from South Africa. A Kaplan-Meier curve from a time-to-event analysis shows an estimate of the proportion of participants who were free from a medically attended RSV-associated LRTI. The hazard ratio and corresponding 95% CIs were obtained from a proportional-hazard model. Tick marks indicate censored data.

FIG. 4 is a table showing subgroup analyses for incidence of medically attended RSV-associated LRTI through 150 days post-dose for the ITT population. a: the relative risk reduction and its 95% CI (mid-P adjusted) were estimated based on exact conditional method using PROC GENMOD with no strata. RRR: relative risk reduction.

FIG. 5 is a panel of graphs showing individual nirsevimab serum concentrations over time, including those participants who developed a medically attended LRTI in the first 150 days. Darker lines represent participants with a breakthrough case of medically attended RSV LRTI prior to Day 151. Day 151 is denoted by dashed vertical grey lines.

FIG. 6 provides further pooled efficacy data demonstrating nirsevimab's efficacy against various severities of medically attended lower respiratory tract infection and against hospitalization due to all-cause respiratory illness. *=Estimated based on Poisson regression with robust variance (including study as a covariate); not corrected for multiplicity; *=Included imputation of missing data; ^{#}=Defined as those cases requiring supplemental oxygen or intravenous fluids (exploratory endpoint); CI=confidence interval.

FIG. 7 provides further pooled efficacy data demonstrating nirsevimab's efficacy against medically attended LRTI across subgroups. CI=confidence interval; RRR=relative risk reduction.

FIG. 8 provides further pooled efficacy data demonstrating nirsevimab's efficacy against medically attended lower respiratory tract infection (LRTI) over 150 days. CI=confidence interval.

FIG. 9 shows pooled efficacy data regarding inpatient health care resource utilization for subjects administered nirsevimab compared to placebo. CPAP=continuous positive airway pressure; HFNC=high-flow nasal cannula; ICU=intensive care unit.

FIG. 10 shows pooled efficacy data regarding outpatient visits and antibiotic use for subjects administered nirsevimab compared to placebo. *=Estimated based on Poisson regression with log follow-up time as offset; *=Calculated as 100× total number of events/total follow-up time (5 months); CI=confidence interval; RRR=relative risk reduction.

FIG. 11A shows RSV neutralising antibody levels and fold-rise from baseline following immunisation with nirsevimab or placebo in Phase IIb. CI=confidence interval; LLOQ=lower limit of quantification; RSV=respiratory syncytial virus; RSV+=confirmed positive RSV infection; RSV-=no confirmed RSV infection or not tested.

FIG. 11B shows RSV neutralising antibody levels and fold-rise from baseline following immunisation with nirsevimab or placebo in Phase III (MELODY). CI=confidence interval; LLOQ=lower limit of quantification; RSV=respiratory syncytial virus; RSV+=confirmed positive RSV infection; RSV-=no confirmed RSV infection or not tested.

FIG. 12A shows geometric mean fold rise (GMFR) of RSV neutralising antibody levels from baseline through day 361 following immunisation with nirsevimab or placebo in Phase IIb. CI=confidence interval; with RSV=with confirmed RSV infection; without RSV=without confirmed RSV infection.

FIG. 12B shows geometric mean fold rise (GMFR) of RSV neutralising antibody levels from baseline through day 361 following immunisation with nirsevimab or placebo in Phase III (MELODY). CI=confidence interval; RSV=respiratory syncytial virus; with RSV=with confirmed RSV infection; without RSV=without confirmed RSV infection.

FIG. 13A shows baseline RSV neutralising antibody levels for patients in Phase IIb and Phase III (MELODY) trials. CI=confidence interval; GMC=geometric mean concentration; NH=Northern Hemisphere; SH=Southern Hemisphere; LLOQ=lower limit of quantification.

FIG. 13B compares baseline RSV neutralising antibody levels across age groups in Phase IIb and Phase III (MELODY) trials. CI=confidence interval; GMC=geometric mean concentration; LLOQ=lower limit of quantification.

FIG. 14 shows a pooled analysis of the efficacy of nirsevimab in reducing RSV LRTI across Phase IIb (Study 3) and Phase III MELODY studies. CI=confidence interval; LRTI=lower respiratory tract infection; MA=medically attended; RRR=relative risk reduction; RSV=respiratory syncytial virus; wkGA=weeks gestational age.

FIG. 15A shows Kaplan-Meier estimates for time to first medically attended RSV LRTI by study by exposure quartile bin. MA RSV LRTI=medically attended RSV lower respiratory tract infection; Q=quartile.

FIG. 15B shows a forest plot of predictors in the pooled final exposure-response model by exposure quartile bin, through day 151. AUC=area under the concentration time curve; CI=confidence interval; HR=hazard ratio.

FIG. 16 shows incidence of medically attended RSV LRTI of different severities through 150 days post dose (ITT population) for all subjects in MELODY trial. ITT population=all infants who underwent randomization; MA RSV LRTI=medically attended RSV lower respiratory tract infection; CI=confidence interval.

### DETAILED DESCRIPTION

The present disclosure provides prophylaxis of RSV infections using nirsevimab, using a single dose per season, wherein the dose may be administered outside of the RSV season. In some embodiments, the prophylactic methods herein prevent RSV-associated LRTI. In some embodiments, the prophylactic methods herein prevent RSV-associated hospitalization. The methods can be used to provide passive immunization of all infants entering their first RSV season and children at a high risk of developing RSV infections (e.g., children with CLD or CHD) entering their first or second RSV season. The present methods may provide a cost-effective opportunity to protect all infants from RSV disease through once-per-RSV-season or pre-RSV-season dosing that effectively mitigates or prevents RSV infection for five or more (e.g., six, seven, eight, nine, ten) months.

The present disclosure identifies an unexpectedly long duration of protective effects from a treatment with nirsevimab, which can persist beyond the length of an RSV season (typically five months in certain geographic areas but potentially longer in other, e.g., more tropical locations). Unexpectedly, the prophylactic protective effects of nirsevimab, as dosed according to the methods herein, have been shown herein to persist beyond 150 days after dosing, e.g., up to at least eight months, as compared to placebo. Due to this surprising advantage, the inventors have discovered that a single dose of nirsevimab may be used to provide protection (e.g., against LRTI or hospitalization associated with RSV) beyond a five month RSV season (the typical length of an RSV season in a temperate climate), and may provide protection throughout even a longer season such as what is observed in some tropical climates, e.g., for as long as at least eight months, or for as long as twelve months. Based on this unexpected period of protection, the inventors have determined that nirsevimab may be dosed before the start of a season (e.g., weeks to months beforehand), enabling the more effective coverage of a larger percentage of eligible patients and/or to allow protection throughout an RSV season in a region having a longer season (e.g., in a tropical or subtropical climate). Thus, a single dose of nirsevimab treatment may be administered before (i.e., at least two, three or more weeks before) the start of the RSV season, regardless of the length of the local RSV season. In some embodiments, a single dose of nirsevimab treatment may be administered one, two, three or more months before the start of the RSV season. In some embodiments, a single dose of nirsevimab treatment may be administered any time of the year, whether inside or outside the RSV season.

The beginning and end of an RSV season can be determined by healthcare professionals and epidemiologists for each geographical region, e.g., by historical trends known to a skilled practitioner, or by evaluating reports on the percentage of patients testing positive for RSV, among other surveillance methods. A typical RSV season in a temperate climate lasts up to about five months (e.g., three, four, or five months). In some circumstances, an RSV season can be longer (e.g., about six, seven, or eight, or more months long) due to local climate or local epidemiologist trends. In some circumstances, an RSV season can be altered in duration due to interruption caused by various factors, such as hygiene and social behavioral changes, e.g., due to hand sanitization, mask wearing and social distancing, for instance as occurred during the COVID-19 pandemic. In both the Northern Hemisphere and the Southern Hemisphere, the RSV season typically starts in the fall (autumn) and ends in the spring. In the United States, the Centers for Disease Control and Prevention analyzes data on RSV activity at the national, regional, and state levels, collected by a surveillance system called the National Respiratory and Enteric Virus Surveillance System (NREVSS). In Europe, the European Centre for Disease Prevention and Control (ECDC) analyzes virological data through The European Surveillance System (TESSy). Generally, the RSV season onset in the United States ranges from mid-September to mid-November, with a season peak ranging from late December to mid-February, and season offset ranging from mid-April to mid-May in all ten U.S. Department of Health and Human Services (HHS) regions, except Florida. Florida has an earlier RSV season onset and longer duration than most regions of the country. In much of the Southern Hemisphere, RSV seasons typically occur between May and September. In tropical or semitropical climates, RSV seasons are frequently associated with the rainy season.

The ability of an RSV prophylactic regimen to protect the greatest number of subjects from disease depends, in part, on the timing of the prophylactic delivery relative to the start of the RSV season. The beginning of the RSV season may depend on multiple factors, including geography and the climate. Therefore, the beginning of the RSV season is often determined by the positivity rate of RSV tests at the local, state, regional, or national level. As used herein, a “positive RSV test” indicates a subject has an RSV infection.

RSV infection (e.g., a positive RSV test) can be determined by diagnostic methods known in the art. See, e.g., Midgley et. al., Determining the Seasonality of Respiratory Syncytial Virus in the United States: The Impact of Increased Molecular Testing. J Infect Dis. 2017 Aug. 1. In some embodiments, an RSV test may be performed on an upper respiratory sample. In some embodiments, an RSV test may be performed on a lower respiratory sample. In some embodiments, RSV infection is determined by a polymerase chain reaction (PCR)-based method. In some embodiments, RSV infection is determined by an antigen-based method. In some embodiments, RSV infection is determined by virus isolation by culture. In some embodiments, RSV infection is determined by serology.

In some embodiments, the beginning of the RSV season is defined by the first two consecutive weeks during which the percentage of positive RSV tests exceeds a threshold. In some embodiments, the threshold is from 3%-13%, e.g., 3%, 4%, 5%, 6%, 7%, 8%, 9%, 10%, 11%, 12% or 13%.

In some embodiments, the beginning of the RSV season is defined by the first two consecutive weeks during which the percentage of PCR tests positive for RSV exceeds a threshold. In some embodiments, the threshold of positive PCR tests is from 3%-13%, e.g., 3%, 5%, 7%, 10%, or 13%. In some embodiments, the threshold of positive PCR tests is 3%. In some embodiments, the threshold of positive PCR tests is 5%. In some embodiments, the threshold of positive PCR tests is 7%. In some embodiments, the threshold of positive PCR tests is 10%. In some embodiments, the threshold of positive PCR tests is 13%.

In some embodiments, the beginning of the RSV season is defined by the first two consecutive weeks during which the percentage of antigen tests positive for RSV exceeds a threshold. In some embodiments, the threshold of positive antigen tests is from 3%-13%, e.g., 3%, 5%, 7%, 10%, or 13%. In some embodiments, the threshold of positive antigen tests is 3%. In some embodiments, the threshold of positive antigen tests is 5%. In some embodiments, the threshold of positive antigen tests is 7%. In some embodiments, the threshold of positive antigen tests is 10%. In some embodiments, the threshold of positive antigen tests is 13%.

Additional normalization processes for measuring threshold positivity rates are known in the art (e.g., moving epidemic model (MEM), retrospective slope 10 (RS10), 10-fold baseline (10FB)). In some embodiments, a moving average of weekly positive tests is used. In some embodiments, a five-week moving average of weekly positive tests is used. In some embodiments, the moving average is normalized to the season peak of 1000 positive RSV tests. In some embodiments, the beginning of the RSV season is the second of two consecutive weeks when the normalized five-week moving average between subsequent weeks increases by at least 10 normalized positive RSV tests per week, provided that the preceding week also met this threshold. In some embodiments, a four-week moving average is defined as the average number of positive tests in the preceding two weeks, current week, and following week. In some embodiments, the beginning of the RSV season is defined by a four-week moving average compared to a preseason baseline. In some embodiments, a preseason baseline is defined as the four-week moving average at week 29. In some embodiments, the beginning of the RSV season is defined as the first of two consecutive weeks when the four-week moving average of positive RSV tests is greater than about 8 times to about 13 times the four-week moving average at a preseason baseline.

In some embodiments, a subject is administered a single dose of nirsevimab before the beginning of the RSV season, wherein the beginning of the RSV season is defined by the first two consecutive weeks during which the average percentage of positive RSV tests over the two week period exceeds a threshold in the range of 3-13%. In some embodiments, a subject is administered a single dose of nirsevimab before the beginning of the RSV season, wherein the beginning of the RSV season is defined by the first two consecutive weeks during which the average percentage of positive RSV tests over the two week period exceeds 3%. In some embodiments, a subject is administered a single dose of nirsevimab before the beginning of the RSV season, wherein the beginning of the RSV season is defined by the first two consecutive weeks during which the average percentage of positive RSV tests over the two week period exceeds 5%. In some embodiments, a subject is administered a single dose of nirsevimab before the beginning of the RSV season, wherein the beginning of the RSV season is defined by the first two consecutive weeks during which the average percentage of positive RSV tests over the two week period exceeds 7%. In some embodiments, a subject is administered a single dose of nirsevimab before the beginning of the RSV season, wherein the beginning of the RSV season is defined by the first two consecutive weeks during which the average percentage of positive RSV tests over the two week period exceeds 10%. In some embodiments, a subject is administered a single dose of nirsevimab before the beginning of the RSV season, wherein the beginning of the RSV season is defined by the first two consecutive weeks during which the average percentage of positive RSV tests over the two week period exceeds 13%. In any of the embodiments of this paragraph, the percentage of positive RSV tests may be determined by a PCR test. In any of the embodiments of this paragraph, the percentage of positive RSV tests may be determined by an antigen test. In some embodiments, an antigen test comprises direct immunofluorescence. In some embodiments, an antigen test is a rapid antigen detection test, e.g., ID NOW™ RSV, Directigen™ RSV, Directigen™ EZ RSV, BinaxNOW™ RSV, BD Veritor™ RSV, Sofia® RSV.

In some embodiments, a subject is administered a single dose of nirsevimab before the beginning of the RSV season, wherein the beginning of the RSV season is defined by the first two consecutive weeks during which the average percentage of positive PCR test over the two week period exceeds a threshold such as 3%. In some embodiments, a subject is administered a single dose of nirsevimab before the beginning of the RSV season, wherein the beginning of the RSV season is defined by the first two consecutive weeks during which the average percentage of positive antigen test over the two week period exceeds 10%. In any of the embodiments of this paragraph, the percentage of positive RSV tests may be determined by an antigen test.

As used herein, administering nirsevimab to a subject “before” the beginning of the RSV season means administering nirsevimab to the subject at least two weeks before the beginning of the RSV season, e.g., as determined according to a method described herein or known in the art. In some embodiments, a subject administered a single dose of nirsevimab before the beginning of the RSV season receives nirsevimab about two weeks before the beginning of the RSV season. In some embodiments, a subject administered a single dose of nirsevimab before the beginning of the RSV season receives nirsevimab about three weeks before the beginning of the RSV season. In some embodiments, a subject administered a single dose of nirsevimab before the beginning of the RSV season receives nirsevimab about four weeks before the beginning of the RSV season. In some embodiments, a subject administered a single dose of nirsevimab before the beginning of the RSV season receives nirsevimab about 1 month before the beginning of the RSV season. In some embodiments, a subject administered a single dose of nirsevimab before the beginning of the RSV season receives nirsevimab about 2 months before the beginning of the RSV season. In some embodiments, a subject administered a single dose of nirsevimab before the beginning of the RSV season receives nirsevimab, about 3 months before the beginning of the RSV season. In some embodiments, a subject administered a single dose of nirsevimab before the beginning of the RSV season receives nirsevimab about 4 months before the beginning of the RSV season. Thus, a subject administered a single dose of nirsevimab before the beginning of the RSV season may receive nirsevimab about 4 months-2 weeks before the beginning of the RSV season

In some embodiments, the beginning of the RSV season is predicted according to the period following the end of the last RSV season (e.g., about 16-28 weeks following the end of the last RSV season), e.g., as determined by a positivity rate (e.g., as measured by PCR) dropping below 3-10%, e.g., 3%. In some embodiments, a next RSV season is predicted to begin about 16 weeks following the end of the last RSV season.

In some embodiments, a subject is administered a single dose of nirsevimab following the end of the last RSV season, at a time predicted to be before the beginning of the next RSV season (e.g., about 16-28 weeks following the end of the last RSV season). In some embodiments, a subject administered a single dose of nirsevimab following the end of the last RSV season receives nirsevimab about 16 weeks following the end of the last RSV season. In some embodiments, a subject administered a single dose of nirsevimab following the end of the last RSV season receives nirsevimab about 17 weeks following the end of the last RSV season. In some embodiments, a subject administered a single dose of nirsevimab following the end of the last RSV season receives nirsevimab about 18 weeks following the end of the last RSV season. In some embodiments, a subject administered a single dose of nirsevimab following the end of the last RSV season receives nirsevimab about 19 weeks following the end of the last RSV season. In some embodiments, a subject administered a single dose of nirsevimab following the end of the last RSV season receives nirsevimab about 20 weeks following the end of the last RSV season. In some embodiments, a subject administered a single dose of nirsevimab following the end of the last RSV season receives nirsevimab about 21 weeks following the end of the last RSV season. In some embodiments, a subject administered a single dose of nirsevimab following the end of the last RSV season receives nirsevimab about 22 weeks following the end of the last RSV season. In some embodiments, a subject administered a single dose of nirsevimab following the end of the last RSV season receives nirsevimab about 23 weeks following the end of the last RSV season. In some embodiments, a subject administered a single dose of nirsevimab following the end of the last RSV season receives nirsevimab about 24 weeks following the end of the last RSV season. In some embodiments, a subject administered a single dose of nirsevimab following the end of the last RSV season receives nirsevimab about 25 weeks following the end of the last RSV season. In some embodiments, a subject administered a single dose of nirsevimab following the end of the last RSV season receives nirsevimab about 26 weeks following the end of the last RSV season. In some embodiments, a subject administered a single dose of nirsevimab following the end of the last RSV season receives nirsevimab about 27 weeks following the end of the last RSV season. In some embodiments, a subject administered a single dose of nirsevimab following the end of the last RSV season receives nirsevimab about 28 weeks following the end of the last RSV season.

In some embodiments, the end to the RSV season is defined by the first week during which the average percentage of positive RSV tests over the one-week period is below a threshold. In some embodiments, the threshold is from 3%-13%, e.g., 3%, 4%, 5%, 6%, 7%, 8%, 9%, 10%, 11%, 12% or 13%. In some embodiments, the threshold of weekly positive RSV tests is 3%. In some embodiments, the threshold of weekly positive RSV tests is 5%. In some embodiments, the threshold of weekly positive RSV tests is 7%. In some embodiments, the threshold of weekly positive RSV tests is 10%. In some embodiments, the threshold of weekly positive RSV tests is 13%.

In some embodiments, the end of the RSV season is defined by the first week during which the average percentage of PCR tests positive for RSV over the one-week period is below a threshold. In some embodiments, the threshold is from 3% to 13%. In some embodiments, the threshold of positive PCR tests is 3%. In some embodiments, the threshold of positive PCR tests is 5%. In some embodiments, the threshold of positive PCR tests is 7%. In some embodiments, the threshold of positive PCR tests is 10%. In some embodiments, the threshold of positive PCR tests is 13%.

In some embodiments, the end of the RSV season is defined by the first week during which the average percentage of antigen tests positive for RSV over the one-week period is below a threshold. In some embodiments, the threshold of positive antigen tests is 3%. In some embodiments, the threshold of positive antigen tests is 5%. In some embodiments, the threshold of positive antigen tests is 7%. In some embodiments, the threshold of positive antigen tests is 10%. In some embodiments, the threshold of positive antigen tests is 13%.

Additional methods for determining the end of an RSV season are known in the art (retrospective slope 10 (RS10), 10-fold baseline (10FB)). In some embodiments, a moving average of weekly positive tests is used. In some embodiments, a five-week moving average of weekly positive tests is used. In some embodiments, the moving average is normalized to the season peak of 1000 positive RSV tests. In some embodiments, the end of the RSV season is the last week that a normalized five-week moving average exceeds an increase in 10 normalized positive RSV tests per week. In some embodiments, a four-week moving average is defined as the average number of positive tests in the preceding two weeks, current week, and following week. In some embodiments, the end of the RSV season is defined by a four-week moving average compared to a preseason baseline. In some embodiments, a preseason baseline is defined as the four-week moving average at week 29. In some embodiments, the end of the RSV season is defined as the last week when the four-week moving average of positive RSV tests is greater than about 8 times to about 13 times the four-week moving average at a preseason baseline.

In some embodiments, a subject is administered a single dose of nirsevimab following the end of the last RSV season, at a time predicted to be before the beginning of the next RSV season. In some embodiments, a subject is administered a single dose of nirsevimab before the beginning of the next RSV season, wherein the next RSV season is defined by a fixed time period following the end of the last RSV season, wherein the end of the last RSV season is defined by the first week during which the average percentage of positive RSV tests over the one-week period is below a threshold. In some embodiments, the threshold is from 3%-13%, e.g., 3%, 4%, 5%, 6%, 7%, 8%, 9%, 10%, 11%, 12% or 13%. In some embodiments, a subject is administered a single dose of nirsevimab before the beginning of the next RSV season, wherein the beginning of the next RSV season is defined by the end of the last RSV season, wherein the end of the last RSV season is the first week during which the average percentage of positive PCR tests over the one-week period is below 3%. In some embodiments, a subject is administered a single dose of nirsevimab before the beginning of the next RSV season, wherein the beginning of the next RSV season is defined by the end of the last RSV season, wherein the end of the last RSV season is the first week during which the average percentage of positive antigen tests over the one-week period is below 10%. In some embodiments, the subject is administered a single dose of nirsevimab about one week following the end of the last RSV season. In some embodiments, the subject is administered a single dose of nirsevimab about two weeks following the end of the last RSV season. In some embodiments, the subject is administered a single dose of nirsevimab about three weeks following the end of the last RSV season. In some embodiments, the subject is administered a single dose of nirsevimab about four weeks following the end of the last RSV season. In some embodiments, the subject is administered a single dose of nirsevimab about 1 month following the end of the last RSV season. In some embodiments, the subject is administered a single dose of nirsevimab about 2 months following the end of the last RSV season. In some embodiments, the subject is administered a single dose of nirsevimab about 3 months following the end of the last RSV season. In some embodiments, the subject is administered a single dose of nirsevimab about 4 months following the end of the last RSV season. In some embodiments, the subject is administered a single dose of nirsevimab about 5 months following the end of the last RSV season. In some embodiments, the subject is administered a single dose of nirsevimab about 6 months following the end of the last RSV season. In some embodiments, the subject is administered a single dose of nirsevimab about 7 months following the end of the last RSV season.

In some embodiments, the beginning of the RSV season is determined by the meteorological season. A meteorological season is dependent upon the regional temperature cycle. For example, in the Northern Hemisphere, meteorological fall (autumn) includes September, October, and November and meteorological spring includes March, April and May. In the Southern Hemisphere, meteorological fall (autumn) includes March, April, and May and meteorological spring includes September, October, and November. In some embodiments, the beginning of the RSV season is in the fall (autumn). In some embodiments, the end of the RSV season is determined by the meteorological season. In some embodiments, the end of the RSV season is in the spring. In some embodiments, a subject is administered nirsevimab after the end of an RSV season or before the start of the next RSV season as determined by meteorological season. In some embodiments, a subject is administered nirsevimab in the spring. In some embodiments, a subject is administered nirsevimab in late spring. In some embodiments, a subject is administered nirsevimab in the summer or early fall (autumn).

In some embodiments, a subject is administered a single dose of nirsevimab before the beginning of the RSV season, wherein the beginning of the RSV season is predicted by a local, state, regional, or national health authority (e.g., ECDC, CDC). In some embodiments, a subject is administered a single dose of nirsevimab after the end of the last RSV season, wherein the end of the RSV season is declared by the local, state, regional, or national health authority.

In some embodiments, a subject is administered a single dose of nirsevimab before the beginning of the RSV season, wherein the beginning of the RSV season is predicted by the NREVSS. In some embodiments, a subject is administered a single dose of nirsevimab after the end of the last RSV season, wherein the end of the RSV season is declared by the NREVSS.

In some embodiments, a subject is administered a single dose of nirsevimab before the beginning of the RSV season, wherein the beginning of the RSV season is predicted by TESSy. In some embodiments, a subject is administered a single dose of nirsevimab after the end of the last RSV season, wherein the end of the RSV season is declared by TESSy.

As used herein, “positivity rate” means the percentage of positive RSV tests over a defined time period. As used herein, “RSV status” or “event status” refers to a subject's test result from an RSV test. As used herein, a “positive RSV test” means a subject has an RSV infection.

As used herein, a “serious RSV disease” or “serious RSV infection” includes lower respiratory tract infection caused by RSV infection (RSV LRTI) or RSV-associated hospitalization. In some embodiments, RSV LRTI is characterized as bronchiolitis or pneumonia. As used herein, “serious RSV” is interchangeable with “severe RSV” and “very serious RSV” is interchangeable with “very severe RSV.”

In some embodiments, a “severe RSV infection” is characterized by at least one of the following: increased respiratory rate (≥60 breaths/min for <2-month-old; ≥50 breaths/min for 2-month old to 6-month old, ≥40 breaths/min for 6-month old to 24-month old); hypoxemia in room air (O₂≤95% at ≤1800 m; O₂≤92% at >1800 m); new-onset apnea; retractions; grunting; nasal flaring; acute hypoxic or ventilatory failure; dehydration due to respiratory distress requiring intravenous hydration; intercostal, subcostal, or supraventricular retractions. In some embodiments, severe RSV infection is characterized by hospitalization for medically attended RSV LRTI.

As used herein, a “very severe RSV infection” is characterized by hospitalization for medically attended RSV LRTI and requiring supplemental oxygen and/or intravenous fluids. In some embodiments, a “very severe RSV infection” is characterized by oxygen saturation (SaO₂)<90%.

As used herein, “prevention”, “protection”, and “providing protection” are used interchangeably. In some embodiments, protection from RSV means inhibiting or mitigating RSV disease. As used herein, “inhibition” includes both partial and full inhibition, e.g., including reduction of one or more symptoms of RSV disease and/or reducing a risk of the RSV disease. In some embodiments, protection from RSV means reducing the severity of symptoms caused by an RSV infection. In some embodiments, providing protection means inhibiting or mitigating lower respiratory tract infection caused by RSV infection (RSV-associated LRTI, particularly medically attended RSV-associated LRTI). In some embodiments, providing protection means inhibiting or mitigating RSV-associated hospitalization. In some embodiments, providing protection means inhibiting or mitigating the contraction of RSV disease (e.g., RSV LRTI). In some embodiments, protection means inhibiting or mitigating severe RSV infection (e.g., severe RSV LRTI, particularly medically attended severe RSV LRTI). In some embodiments, protection means inhibiting or mitigating very severe RSV infection (e.g., very severe RSV LRTI, particularly medically attended very severe RSV LRTI). In some embodiments, protection means reducing the risk of RSV disease. In some embodiments, protection means reducing the risk of lower respiratory tract infection caused by RSV infection (RSV-associated LRTI, particularly medically attended RSV-associated LRTI). In some embodiments, protection means reducing the risk of RSV-associated hospitalization. In some embodiments, protection means reducing the risk of the contraction of RSV. In some embodiments, protection means reducing the risk of severe RSV (e.g., severe RSV LRTI, particularly medically attended severe RSV LRTI). In some embodiments, protection means reducing the risk of very severe RSV (e.g., very severe RSV LRTI, particularly medically attended very severe RSV LRTI).

In some embodiments, protection means reducing the risk of all-cause LRTI, particularly medically attended all-cause LRTI. In some embodiments, protection means reducing the risk of all-cause LRTI hospitalizations.

Protection may be understood as relative to a subject who was not administered nirsevimab. In some embodiments, protection from RSV means inhibiting or mitigating RSV disease compared to a subject who was not administered nirsevimab. In some embodiments, protection from RSV means inhibiting or mitigating lower respiratory tract infection caused by RSV infection (RSV-associated LRTI, particularly medically attended RSV-associated LRTI) compared to a subject who was not administered nirsevimab. In some embodiments, protection from RSV means inhibiting or mitigating RSV-associated hospitalization compared to a subject who was not administered nirsevimab. In some embodiments, protection from RSV means inhibiting or mitigating the contraction of RSV compared to a subject who was not administered nirsevimab. In some embodiments, protection from RSV means inhibiting or mitigating severe RSV (e.g., severe RSV LRTI, particularly medically attended severe RSV LRTI) compared to a subject who was not administered nirsevimab. In some embodiments, protection from RSV means inhibiting or mitigating very severe RSV infection (e.g., very severe RSV LRTI, particularly medically attended very severe RSV LRTI) compared to a subject who was not administered nirsevimab.

In some embodiments, protection from RSV means reducing the risk of RSV disease compared to a subject who was not administered nirsevimab. In some embodiments, protection from RSV means reducing the risk of lower respiratory tract infection caused by RSV infection (RSV-associated LRTI, particularly medically attended RSV-associated LRTI) compared to a subject who was not administered nirsevimab. In some embodiments, protection from RSV means reducing the risk of RSV-associated hospitalization compared to a subject who was not administered nirsevimab. In some embodiments, protection from RSV means reducing the risk of the contraction of RSV compared to a subject who was not administered nirsevimab. In some embodiments, protection from RSV means reducing the risk of severe RSV (e.g., severe RSV LRTI, particularly medically attended severe RSV LRTI) compared to a subject who was not administered nirsevimab. In some embodiments, protection from RSV means reducing the risk of very severe RSV infection (e.g., very severe RSV LRTI, particularly medically attended very severe RSV LRTI) compared to a subject who was not administered nirsevimab.

As used herein, the terms “immunized” or “immunization,” in reference to the administration of nirsevimab, encompass passive immunization.

As used herein, “RSV-associated LRTI,” “RSV-confirmed LRTI,” and “RSV LRTI” are used interchangeably. In some embodiments, the RSV LRTI is medically attended RSV LRTI (MA RSV LRTI or RSV MALRTI).

As used herein, RSV lower respiratory tract disease (RSV LRTD) is synonymous with RSV LRTI. Similarly, medically attended RSV LRTD is synonymous with medically attended RSV LRTI. LRTD and LRTI are not necessarily synonymous outside of the context of RSV infection.

As used herein, “all-cause LRTI” and “all-cause medically attended LRTI,” refer to all cases of LRTI or medically attended LRTI, respectively, and include but are not limited to RSV LRTI (for all-cause LRTI) or MA RSV LRTI (for all-cause MA LRTI). As used herein, “all-cause respiratory illness” encompasses any case of respiratory illness, including but not limited to a respiratory illness caused by RSV infection. As used herein, “all-cause LRTI hospitalization” encompasses any LRTI hospitalization, including but not limited to hospitalization due to RSV LRTI.

As used herein, a timepoint “outside the RSV season” refers to a timepoint that is not within the RSV season, including the first bi-week (i.e., two consecutive weeks) in which a positivity rate is above a set threshold for determining the beginning of the season. In some embodiments, a time outside the RSV season is a time more than about two, three, or four weeks, or more than about one, two, three, four, five, six, or seven months before commencement of the RSV season. As used herein, “beginning” in reference to an RSV season means the “start” or “commencement” of the season, as these terms are used interchangeably herein. In some embodiments, the beginning of the RSV season is defined by a two-week period, e.g., in which a positivity rate exceeds a threshold. In some embodiments, the beginning of the RSV season is defined by a starting date. As used herein, “RSV season” can refer to a season of RSV-A, RSV-B, or both.

As used herein, “subtropical” and “semitropical” are used interchangeably.

As used herein, an “infant” subject is a human subject that is one year (12 months) of age or younger. As used herein, a “pediatric” subject is a human child subject that is older than one year of age. In some embodiments, a pediatric subject is a human subject that is older than one year (12 months) of age and up to or including 24 months of age.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B,” when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When a number is recited, either alone or as part of a numerical range, it should be understood that the numerical value can vary above and below the stated value by a variance that is reasonable for the value described, as recognized by one of skill in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context. As used herein, the term “approximately” or “about” as applied to a number of weeks means +/−3 days. As used herein, the term “approximately” or “about” as applied to a number of months means +/−2 weeks.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. Although a number of documents are cited herein, this citation does not constitute an admission that any of these documents forms part of the common general knowledge in the art. All publications and other references mentioned herein are incorporated by reference in their entirety.

### I. Nirsevimab and Pharmaceutical Compositions Thereof

In various embodiments, the antibody administered according to the methods and uses disclosed herein is nirsevimab. Nirsevimab (aka MEDI8897) is a recombinant human immunoglobulin G₁ kappa (IgGIK) monoclonal antibody (mAb) directed against the prefusion conformation of the RSV F protein. See, e.g., U.S. Pat. No. 10,689,437, which is hereby incorporated by reference in its entirety. The antibody binds both the F1 and F2 subunits of the F protein at a highly conserved epitope, locking RSV F in the prefusion conformation to block fusion and viral entry into the host cell. The heavy chain of nirsevimab has the following sequences, with the complementarity-determining regions (CDRs) boxed and the variable domain (V_{H}) italicized:

(SEQ ID NO: 1)

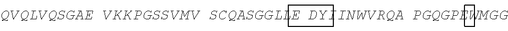

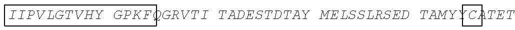

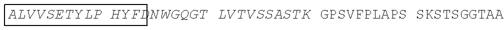

LGCLVKDYFP EPVTVSWNSG ALTSGVHTFP AVLQSSGLYS LSSVVTVPSS
SLGTQTYICN VNHKPSNTKV DKRVEPKSCD KTHTCPPCPA PELLGGPSVF
LFPPKPKDTL YITREPEVTC VVVDVSHEDP EVKENWYVDG VEVHNAKTKP
REEQYNSTYR VVSVLTVLHQ DWLNGKEYKC KVSNKALPAP IEKTISKAKG
QPREPQVYTL PPSREEMTKN QVSLTCLVKG FYPSDIAVEW ESNGQPENNY
KTTPPVLDSD GSFFLYSKLT VDKSRWQQGN VESCSVMHEA LHNHYTQKSL
SLSPGK

In some embodiments, nirsevimab has the heavy chain sequence of SEQ ID NO: 1. In some embodiments, nirsevimab has the heavy chain sequence of SEQ ID NO: 1 lacking the C-terminal lysine (K456) (SEQ ID NO: 11). In some embodiments, nirsevimab comprises a mixture of antibodies comprising the heavy chain sequence SEQ ID NO: 1 and antibodies comprising the heavy chain sequence SEQ ID NO: 11. In the above sequence, the amino acid sequence of the V_{H} is represented by SEQ ID NO: 2, and the amino acid sequences of the heavy chain CDRs (HCDRs) are represented by SEQ ID NOs: 3-5, respectively. The heavy chain contains a triple amino acid substitution (“YTE”), shown above with underlines and boldface. This YTE triple mutation (M252Y/S254T/T256E; EU numbering) was made relative to wildtype human IgGi in the fragment crystallizable (Fc) region. While the heavy chain of nirsevimab contains a triple amino acid substitution (“YTE”), which has been shown to increase antibody half-life by a few weeks, the increased duration of efficacy past five months was unanticipated.

The light chain of nirsevimab has the following sequences, with the CDRs boxed and the variable domain (V_{L}) italicized:

(SEQ ID NO: 6)

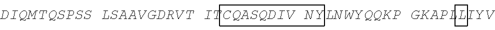

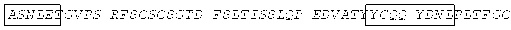

GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSEN RGEC

In the above sequences, the amino acid sequence of the V_{L} is represented by SEQ ID NO: 7, and the amino acid sequences of the light chain CDRs (LCDRs) are represented by SEQ ID NOs: 8-10, respectively.

Nirsevimab neutralizes RSV by binding the prefusion conformation of the RSV F protein at a site distinct from that bound by palivizumab. In preclinical studies, nirsevimab was >150-fold more potent than palivizumab in vitro and approximately 9-fold more potent than palivizumab in vivo in the cotton rat model (Zhu et al., Sci Transl Med. (2017) 9: eaaj. 1928).

The antibody is provided to subjects in need thereof as a pharmaceutical composition. The pharmaceutical composition may comprise a pharmaceutically acceptable carrier, diluent and/or excipient, formulated for intramuscular injection. In some embodiments, the composition is a sterile, preservative-free liquid solution containing a buffer agent (e.g., histidine), an amino acid (e.g., arginine or methionine), a polyol (e.g., sucrose), and a surfactant (e.g., polysorbate 80 or polysorbate 20). In further embodiments, the composition comprises 100 mg/mL nirsevimab, 30 mM histidine/histidine-HCl, 80 mM arginine-HCl, 120 mM sucrose, and 0.02%-0.04% (w/v) polysorbate 80, pH 6.0. Additional embodiments of compositions comprising nirsevimab are described in International Application No. PCT/US2018/020264, which is hereby incorporated by reference in its entirety.

In some embodiments of the pharmaceutical composition, nirsevimab is present at a concentration of about 25 mg/ml or greater (e.g., about 25 mg/ml to about 250 mg/ml). In some embodiments of the pharmaceutical composition, nirsevimab is present at a concentration of about 50 mg/ml or greater (e.g., about 50 mg/ml to about 250 mg/ml). In some embodiments of the pharmaceutical composition, nirsevimab is present at a concentration of about 50 mg/ml to about 200 mg/ml. In some embodiments, nirsevimab is present at a concentration of about 75 mg/ml or greater (e.g., about 75 mg/ml to about 250 mg/ml). In some embodiments, nirsevimab is present at a concentration of about 100 mg/ml or greater. In some embodiments, nirsevimab is present at a concentration of about 100 mg/ml to about 165 mg/ml. In some embodiments, nirsevimab is present at a concentration of about 100 mg/ml.

In some embodiments, the pharmaceutical composition has a pH ranging from about pH 5.5 to about pH 6.5. In some embodiments, the pH has a range of about pH 5.7 to about pH 6.3. In some embodiments, the pH has a range of about pH 5.7 to about pH 6.1. In some embodiments, the pH is about 5.8. In some embodiments, the pH is about 6.0.

In some embodiments of the pharmaceutical composition, a salt is present at a concentration of about 75 mM to about 100 mM. In some embodiments, the salt is present at a concentration of about 75 mM or about 80 mM. In some embodiments, the salt is arginine hydrochloride, for example at a concentration of about 75 mM to about 100 mM, optionally at a concentration of about 80 mM. In some embodiments of the pharmaceutical composition, a sugar (e.g., sucrose) is present at concentration of about 100 mM to about 140 mM, optionally at a concentration of about 120 mM.

In some embodiments of the pharmaceutical composition, the composition comprises one or more buffers. In some embodiments, the buffer comprises histidine hydrochloride. In some embodiments, the concentration of the buffer is about 10 mM to about 50 mM, optionally about 30 mM.

In some embodiments of the pharmaceutical composition, the composition comprises a surfactant. In some embodiments, the surfactant is a polysorbate, including for example, polysorbate-80. In some embodiments, the surfactant is present at a concentration of about 0.02%-0.04% (w/v). In an embodiment, the surfactant is present at a concentration of about 0.02%. In another embodiment, the surfactant is present at a concentration of about 0.04%.

In some embodiments, the pharmaceutical composition is provided in a single-unit vial or multi-unit vial. Each unit may contain 50, 100, or 200 mg of nirsevimab. In some embodiments, in a single-unit container (e.g., a vial or a pre-filled injector or syringe), the container contains a nominal fill volume of 0.5 mL of the aforementioned 100 mg/mL pharmaceutical composition. In other embodiments, in a single-unit container (e.g., a vial or a pre-filled injector or syringe), the container contains a nominal fill volume of 1 mL of the aforementioned 100 mg/mL pharmaceutical composition. In a multi-unit container, the container may contain a nominal fill volume of multiples of 0.5 mL of the aforementioned 100 mg/mL pharmaceutical composition. In other embodiments, the multi-unit container may contain a nominal fill volume of multiples of 0.5 mL and/or 1 mL of the aforementioned 100 mg/mL pharmaceutical composition.

In some embodiments, articles of manufacture (e.g., kits) are provided, comprising single-unit containers or multi-unit containers comprising pharmaceutical compositions of nirsevimab (e.g., the aforementioned 100 mg/mL composition). In some embodiments, articles of manufacture may further comprise instructions for use.

In some embodiments, a pharmaceutical composition comprising nirsevimab is for use in any method described herein. In some embodiments, nirsevimab is used in the manufacture of a medicament for use in any method described herein. In some embodiments, an article of manufacture comprising nirsevimab is used in any method described herein.

The subject may be injected intramuscularly or subcutaneously with one or more units of the composition depending on their weight. Intramuscular or subcutaneous administration may be inseparable or difficult to distinguish for certain small subjects (e.g., infants) and in such subjects, intramuscular and/or subcutaneous administration may be acceptable. For example, infants who weigh less than 5 kg may be injected intramuscularly or subcutaneously with one unit (50 mg per unit) of nirsevimab provided in such a pharmaceutical composition, while infants who weigh 5 kg or more may be injected intramuscularly or subcutaneously with 100 mg (e.g., two 50 mg units, one 100 mg unit) of nirsevimab provided in such a pharmaceutical composition. A pediatric subject who is older than one year of age and/or entering their second RSV season may be injected intramuscularly or subcutaneously with 200 mg (e.g., four 50 mg units, two 100 mg units, or one 200 mg unit) of nirsevimab. In some embodiments, the pediatric subject injected intramuscularly or subcutaneously with 200 mg of nirsevimab is at high risk of developing an RSV infection, such as RSV LRTI.

### II. Methods of Use

Nirsevimab may be injected (e.g., subcutaneously or, preferably, intramuscularly) into a human subject to prevent an RSV infection, particularly an RSV LRTI. In some embodiments, nirsevimab may be injected (e.g., subcutaneously or, preferably, intramuscularly) into an infant or pediatric subject to prevent an RSV infection, particularly an RSV LRTI. In some embodiments, nirsevimab may be injected into an infant or pediatric subject to reduce the risk of LRTI (e.g., bronchitis or pneumonia) in the subject, e.g., in a prophylaxis regimen. In some embodiments, nirsevimab may be injected into an infant or pediatric subject to reduce the risk of medically attended LRTI. In some embodiments, nirsevimab may be injected into an infant or pediatric subject to reduce the risk of severe RSV infection, particularly severe RSV LRTI. In some embodiments, nirsevimab may be injected into an infant or pediatric subject to reduce the risk of very severe RSV infection, particularly very severe RSV LRTI. In some embodiments, nirsevimab may be injected into an infant or pediatric subject to reduce the risk of hospitalization, e.g., in a prophylaxis regimen. In some embodiments, nirsevimab may be injected into an infant or pediatric subject to reduce the incidence of LRTI. In some embodiments, nirsevimab may be injected into an infant or pediatric subject to reduce the incidence of medically attended LRTI. In some embodiments, nirsevimab may be injected into an infant or pediatric subject to reduce the incidence of severe RSV infection, particularly severe RSV LRTI. In some embodiments, nirsevimab may be injected into an infant or pediatric subject to reduce the incidence of very severe RSV infection, particularly very severe RSV LRTI. In some embodiments, nirsevimab may be injected into an infant or pediatric subject to reduce the incidence of hospitalization.

In some embodiments, nirsevimab may be injected into an infant or pediatric subject in a method of obtaining protection from a respiratory syncytial virus (RSV) infection and/or RSV disease for more than five months. In some embodiments, nirsevimab may be injected into an infant or pediatric subject in a method of obtaining protection from a respiratory syncytial virus (RSV) infection and/or RSV disease for at least eight months, optionally about 12 months. In some embodiments, nirsevimab may be injected into an infant or pediatric subject in a method of obtaining protection from a respiratory syncytial virus (RSV) infection and/or RSV disease for at least one RSV season. In some embodiments, the RSV infection and/or RSV disease comprises RSV LRTI. In some embodiments, the RSV infection and/or RSV disease comprises medically attended RSV LRTI. In some embodiments, the RSV infection and/or RSV disease comprises hospitalization for RSV LRTI.

In various embodiments, a subject may be injected with a single dose (e.g., 50, 100, or 200 mg) of nirsevimab before an RSV season. In some embodiments, the RSV injection may be given any time of the year. In some embodiments, the injection may take place at or shortly after birth. In some embodiments, the injection may take place before the beginning of the RSV season. In some embodiments, a subject administered a single dose of nirsevimab before the beginning of the RSV season receives nirsevimab about two weeks before the beginning of the RSV season. In some embodiments, a subject administered a single dose of nirsevimab before the beginning of the RSV season receives nirsevimab about three weeks before the beginning of the RSV season. In some embodiments, a subject administered a single dose of nirsevimab before the beginning of the RSV season receives nirsevimab about four weeks before the beginning of the RSV season. In some embodiments, a subject administered a single dose of nirsevimab before the beginning of the RSV season receives nirsevimab about 1 month before the beginning of the RSV season. In some embodiments, a subject administered a single dose of nirsevimab before the beginning of the RSV season receives nirsevimab about 2 months before the beginning of the RSV season. In some embodiments, a subject administered a single dose of nirsevimab before the beginning of the RSV season receives nirsevimab about 3 months before the beginning of the RSV season. In some embodiments, a subject administered a single dose of nirsevimab before the beginning of the RSV season receives nirsevimab about 4 months before the beginning of the RSV season. Thus, a subject administered a single dose of nirsevimab before the beginning of the RSV season may receive nirsevimab about 4 months to about 2 weeks before the beginning of the RSV season.

In some embodiments, a subject is administered a single dose of nirsevimab following the end of the last RSV season, at a time predicted to be before the beginning of the next RSV season (e.g., about 16-28 weeks following the end of the last RSV season). In some embodiments, a subject administered a single dose of nirsevimab following the end of the last RSV season receives nirsevimab about 16 weeks following the end of the last RSV season. In some embodiments, a subject administered a single dose of nirsevimab following the end of the last RSV season receives nirsevimab about 17 weeks following the end of the last RSV season. In some embodiments, a subject administered a single dose of nirsevimab following the end of the last RSV season receives nirsevimab about 18 weeks following the end of the last RSV season. In some embodiments, a subject administered a single dose of nirsevimab following the end of the last RSV season receives nirsevimab about 19 weeks following the end of the last RSV season. In some embodiments, a subject administered a single dose of nirsevimab following the end of the last RSV season receives nirsevimab about 20 weeks following the end of the last RSV season. In some embodiments, a subject administered a single dose of nirsevimab following the end of the last RSV season receives nirsevimab about 21 weeks following the end of the last RSV season. In some embodiments, a subject administered a single dose of nirsevimab following the end of the last RSV season receives nirsevimab about 22 weeks following the end of the last RSV season. In some embodiments, a subject administered a single dose of nirsevimab following the end of the last RSV season receives nirsevimab about 23 weeks following the end of the last RSV season. In some embodiments, a subject administered a single dose of nirsevimab following the end of the last RSV season receives nirsevimab about 24 weeks following the end of the last RSV season. In some embodiments, a subject administered a single dose of nirsevimab following the end of the last RSV season receives nirsevimab about 25 weeks following the end of the last RSV season. In some embodiments, a subject administered a single dose of nirsevimab following the end of the last RSV season receives nirsevimab about 26 weeks following the end of the last RSV season. In some embodiments, a subject administered a single dose of nirsevimab following the end of the last RSV season receives nirsevimab about 27 weeks following the end of the last RSV season. In some embodiments, a subject administered a single dose of nirsevimab following the end of the last RSV season receives nirsevimab about 28 weeks following the end of the last RSV season.

In another embodiment, the injection may take place at the beginning of or during the RSV season when the RSV season is long (e.g., longer than five months, such as six, seven, eight, nine or ten months). In further embodiments, the injection need not be limited to the beginning of the RSV season or during the RSV season when the RSV season is long (e.g., longer than five months, such as six, seven, eight, nine or ten months). In some embodiments, even if the RSV season is longer than five months (e.g., six, seven, eight, nine, or ten months), a single dose of nirsevimab, given outside or within (e.g., during or at the beginning of) the RSV season, provides protection against RSV infection in a subject for at least the entire RSV season. In certain embodiments, a single dose of nirsevimab, given outside or within (e.g., during or at the beginning of) the RSV season, provides protection against RSV in a subject for more than one RSV season (e.g., for two RSV seasons) or for at least one RSV season extended by an interruption. In some embodiments, protection comprises preventing an RSV infection. In some embodiments, protection comprises reducing the risk of an RSV infection. In some embodiments, protection comprises preventing RSV-associated LRTI. In some embodiments, protection comprises reducing the risk of an RSV-associated LRTI. In some embodiments, protection comprises preventing RSV-associated hospitalization. In some embodiments, protection comprises reducing the risk of an RSV-associated hospitalization.

In some embodiments, nirsevimab may be administered to an infant or pediatric subject before their first RSV season. In some embodiments, nirsevimab may be administered for the first time to a subject before the subject's second or subsequent RSV season. In some embodiments, nirsevimab may be administered to a subject before the subject's second or subsequent RSV season. In such embodiments, nirsevimab may be administered for the first time. In some embodiments, nirsevimab may be administered a second time to a subject before the subject's second or subsequent RSV season.

In some embodiments, a subject is administered a first dose of 50 mg or 100 mg of nirsevimab before or during their first RSV season. In some embodiments, the subject is administered a 50 mg dose if the subject weighs less than 5 kg at the time of administration and a 100 mg dose if the subject weighs 5 kg or more at the time of administration. In some embodiments, the subject is administered a second dose of 200 mg of nirsevimab before or during their second RSV season.

In some embodiments, a subject who is at high risk of developing an RSV infection, such as RSV LRTI (a “high-risk subject” or “a subject vulnerable to severe RSV disease”), is administered a first dose of 50 mg or 100 mg of nirsevimab before or during their first RSV season. In some embodiments, the high-risk subject is administered a 50 mg dose if the subject weighs less than 5 kg at the time of administration and a 100 mg dose if the subject weighs 5 kg or more at the time of administration before or during their first RSV season. In some embodiments, the high-risk subject is administered a second dose of 200 mg of nirsevimab before or during their second RSV season. In some embodiments, the high-risk subject has premature birth, chronic lung disease (CLD), congenital heart disease (CHD), a suppressed or compromised immune system, an immunodeficiency, a neuromuscular disorder, Down's syndrome, a congenital airway anomaly, and/or cystic fibrosis.

In some embodiments, a single dose of nirsevimab may be administered to an infant or pediatric subject not more than once per RSV season. In such embodiments, a single dose of nirsevimab may be administered at the beginning of the RSV season or during the RSV season. In some embodiments, a single dose of nirsevimab may be administered to an infant or pediatric subject not more than once per year. In such embodiments, a single dose of nirsevimab may be administered at the beginning of the RSV season or during the RSV season. Accordingly, in some embodiments, nirsevimab may be administered to subjects born outside of the RSV season. In another embodiment, nirsevimab may be administered to subjects who are experiencing an interrupted RSV season. For example, an RSV season may be interrupted by hygiene or social behavioral changes, e.g., due to more frequent hand sanitization, mask wearing and social distancing, such as occurred during the COVID-19 pandemic. In such embodiments, the interrupted RSV season resumes when, for example, the hygiene or social behavioral changes ends. In some embodiments, nirsevimab may be administered to a subject before their first RSV season, wherein the subject's first RSV season occurs after the subject has entered the second year of life (i.e., is at least 1 year old). For example, a subject's first RSV season may not occur until the subject has entered their second year of life, for instance, due to a global pandemic, due to an interrupted RSV season, or due to the relocation of the subject to a different climate. In such embodiments, the first RSV season may occur during the subject's first year of life or may be delayed past the first year of life. In a further embodiment, nirsevimab may be administered to subjects experiencing an RSV season that is longer than five months (e.g., six, seven, eight, nine, or ten months). In such embodiments, a single dose (e.g., 50, 100, or 200 mg) of nirsevimab will suffice to provide protection against RSV infections (e.g., RSV-associated LRTI, hospitalization) in the immunized individual for at least one RSV season or for at least one RSV season extended by an interruption.

In some embodiments, nirsevimab is administered to a subject in an amount effective to yield serum AUC_{0-∞}, of greater than 10 day mg/mL, 11 day mg/mL, 12 day mg/mL, 13 day mg/mL, or 14 day mg/mL. In some embodiments, nirsevimab is administered to a subject in an amount effective to yield serum AUC_{0-∞}, of greater than 12.8 day·mg/mL.

In some embodiments, the subject exhibits serum AUC_{0-∞}, of greater than 10 day mg/mL, 11 day mg/mL, 12 day mg/mL, 13 day mg/mL, or 14 day mg/mL. In some embodiments, the subject exhibits serum AUC_{0-∞}, of greater than 12.8 day mg/mL.

In some embodiments, a single dose of nirsevimab is administered to a subject wherein the amount of nirsevimab in the single dose is effective to yield elevated RSV neutralizing antibody (Nab) levels in the subject after administration compared to the subject's RSV Nab levels at the time of administration. In some embodiments, the nirsevimab in the single dose is in an amount effective to yield elevated RSV Nab levels in the subject at 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, or 361 days after administration compared to the subject's RSV Nab levels at the time of administration. In some embodiments, the nirsevimab in the single dose is in an amount effective to yield elevated RSV Nab levels in the subject at 31, 91, 151, or 361 days after administration compared to the subject's RSV Nab levels at the time of administration. In some embodiments, the nirsevimab in the single dose is in an amount effective to yield elevated RSV Nab levels in the subject at about 5, about 6, about 7, about 8, about 9, about 10, about 11, or about 12 months after administration compared to the subject's RSV Nab levels at the time of administration. In some embodiments, the nirsevimab in the single dose is in an amount effective to yield RSV Nab levels in the subject at about 5, about 6, about 7, about 8, about 9, about 10, about 11, or about 12 months after administration that are >3-, 4-, 5-, 6-, 7-, 8-, 9-, 10-, 11-, 12-, 13-, 14-, 15-, 16-, 17-, 18-, 19-, 20-, 21-, 22-, 23-, 24-, or 25-fold higher than the subject's RSV Nab levels at the time of administration. In some embodiments, the nirsevimab in the single dose is in an amount effective to yield RSV Nab levels in the subject at about 12 months after administration that are >5-fold higher than the subject's RSV Nab levels at the time of administration. In some embodiments, the nirsevimab in the single dose is in an amount effective to yield RSV Nab levels in the subject at about 12 months after administration that are >7-fold higher than the subject's RSV Nab levels at the time of administration.

In some embodiments, the nirsevimab in the single dose is in an amount effective to yield elevated levels of RSV Nab in the subject at about 5, about 6, about 7, about 8, about 9, about 10, about 11, or about 12 months after administration compared to RSV Nab levels in a subject with a confirmed RSV infection and who was not administered nirsevimab. In some embodiments, the nirsevimab in the single dose is in an amount effective to yield RSV Nab levels in the subject at about 5, about 6, about 7, about 8, about 9, about 10, about 11, or about 12 months after administration that are >1-, 2-, 3-, 4-, or 5-fold higher than RSV Nab levels in a subject with a confirmed RSV infection and who was not administered nirsevimab. In some embodiments, the nirsevimab in the single dose is in an amount effective to yield RSV Nab levels in the subject at about 12 months after administration that are >3-fold higher than RSV Nab levels in a subject with a confirmed RSV infection and who was not administered nirsevimab.

In some embodiments, the subject is administered a single dose of nirsevimab comprising 50 mg, 100 mg, or 200 mg nirsevimab. In some embodiments, the subject exhibits elevated RSV Nab levels after administration of the single dose of nirsevimab compared to the subject's RSV Nab levels at the time of administration. In some embodiments, the subject exhibits elevated RSV Nab levels at 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, or 361 days after administration of the single dose of nirsevimab compared to the subject's RSV Nab levels at the time of administration. In some embodiments, the subject exhibits elevated RSV Nab levels at 31, 91, 151, or 361 days after administration of the single dose of nirsevimab compared to the subject's RSV Nab levels at the time of administration. In some embodiments, the subject exhibits elevated RSV Nab levels at about 5, about 6, about 7, about 8, about 9, about 10, about 11, or about 12 months after administration of the single dose of nirsevimab compared to the subject's RSV Nab levels at the time of administration. In some embodiments, the subject's RSV Nab levels at about 5, about 6, about 7, about 8, about 9, about 10, about 11, or about 12 months after administration of the single dose of nirsevimab are >3-, 4-, 5-, 6-, 7-, 8-, 9-, 10-, 11-, 12-, 13-, 14-, 15-, 16-, 17-, 18-, 19-, 20-, 21-, 22-, 23-, 24-, or 25-fold higher than the subject's RSV Nab levels at the time of administration. In some embodiments, the subject's RSV Nab levels at about 12 months after administration of the single dose of nirsevimab are >5-fold higher than the subject's RSV Nab levels at the time of administration. In some embodiments, the subject exhibits elevated levels of RSV Nab at about 12 months after administration of the single dose of nirsevimab that are >7-fold higher than the subject's RSV Nab levels at the time of administration. In some embodiments, the subject exhibits elevated levels of RSV Nab at about 5, about 6, about 7, about 8, about 9, about 10, about 11, or about 12 months after administration of the single dose of nirsevimab compared to RSV Nab levels in a subject with a confirmed RSV infection and who was not administered nirsevimab. In some embodiments, the subject exhibits elevated levels of RSV Nab at about 5, 6, 7, 8, 9, 10, 11, or 12 months after administration of the single dose of nirsevimab that are >1-, 2-, 3-, 4-, or 5-fold higher than a subject with a confirmed RSV infection who was not administered nirsevimab. In some embodiments, the subject exhibits elevated levels of RSV Nab at about 12 months after administration of the single dose of nirsevimab that are >3-fold higher than a subject with a confirmed RSV infection who was not administered nirsevimab.

A subject in need of nirsevimab treatment may be any subject susceptible to RSV infections. In some embodiments, the subject is an infant, e.g., an infant ≤3 months of age, >3 to ≤6 months of age, or >6 months of age (e.g., >6 months and ≤12 months). In some embodiments, the infant is a late preterm infant or a full-term (“term”) infant (e.g., born with a gestational age of ≥35 weeks), optionally wherein the infant is healthy. In some embodiments, the infant is a preterm infant born with a gestational age of ≥29 weeks, optionally wherein the infant is healthy. In some embodiments, the infant is born with a gestational age of <29 weeks.

In some embodiments, the subject is a child born prematurely who is in their first or second year of life, or a child who remains at risk of RSV infections beyond their second year of life. In some embodiments, the subject is <5 kg. In some embodiments, the subject is ≥5 kg.

In some embodiments, the subject is at a high risk of developing an RSV infection (e.g., RSV-associated LRTI). Levels of risks may be determined by healthcare professionals. See, e.g., guidelines provided by the American Academy of Pediatrics. For example, children with any of the following underlying conditions are considered at high risk:
children younger than 2 years old with chronic lung disease (CLD) or congenital heart disease (CHD);
children with suppressed immune systems; and
children who have neuromuscular disorders, including those who have difficulty swallowing or clearing mucus secretions and a gestational age of 35 or fewer weeks.

Premature babies are at an increased risk for CLD due to the immaturity of their lung at birth and lung injury resulting from treatments such as use of a mechanical ventilator and/or use of a high concentration of oxygen. Infants with CLD are at a particularly high risk of morbidity due to RSV infection. In some embodiments, the subject to be immunized may have CLD. In some embodiments, the subject to be immunized may be a child (e.g., an infant or pediatric subject) who has CLD.

Children with CHD include those having hemodynamically significant CHD, which may adversely affect pulmonary blood flow. Children with hemodynamically significant CHD have a greater rate of RSV-related hospitalization. In some embodiments, the subject to be immunized may have CHD. In some embodiments, the subject to be immunized may be a child (e.g., an infant or pediatric subject) who has CHD.

In some embodiments, the subject to be immunized may have Down's syndrome. Children with Down's syndrome have been reported to have a significantly higher risk of severe RSV infection than children without Down's syndrome (Beckhaus et al., Pediatrics (2018) 142 (3): e20180225). In some embodiments, the subject to be immunized may be a child (e.g., an infant or pediatric subject) who has Down's syndrome.

In some embodiments, the subject to be immunized may have cystic fibrosis. In some embodiments, the subject to be immunized may be a child (e.g., an infant or pediatric subject) who has cystic fibrosis.

In some embodiments, the subject to be immunized may be immunocompromised. In some embodiments, the subject to be immunized may be a child (e.g., an infant or pediatric subject) who is immunocompromised. In some embodiments, the subject to be immunized may be immunodeficient (including combined, antibody, or other etiology for the immunodeficiency). In some embodiments, the subject to be immunized may be a child (e.g., an infant or pediatric subject) who is immunodeficient. In some embodiments, the subject to be immunized may have a primary immunodeficiency. In some embodiments, the subject to be immunized may be a child (e.g., an infant or pediatric subject) who has a primary immunodeficiency. In some embodiments, the subject to be immunized may have a human immunodeficiency virus infection. In some embodiments, the subject to be immunized may be a child (e.g., an infant or pediatric subject) who has a human immunodeficiency virus infection. In some embodiments, the subject to be immunized may have a history of organ or bone marrow transplantation. In some embodiments, the subject to be immunized may be a child (e.g., an infant or pediatric subject) who has a history of organ or bone marrow transplantation. In some embodiments, the subject to be immunized receives immunosuppressive chemotherapy. In some embodiments, the subject to be immunized may be a child (e.g., an infant or pediatric subject) who immunized receives immunosuppressive chemotherapy. In some embodiments, the subject to be immunized receives systemic high-dose corticosteroid therapy. In some embodiments, the subject to be immunized may be a child (e.g., an infant or pediatric subject) who receives systemic high-dose corticosteroid therapy. In some embodiments, the subject to be immunized receives other immunosuppressive therapy. In some embodiments, the subject to be immunized may be a child (e.g., an infant or pediatric subject) who receives other immunosuppressive therapy. In some embodiments, the subject to be immunized may have a congenital airway anomaly. In some embodiments, the subject to be immunized may be a child (e.g., an infant or pediatric subject) who has a congenital airway anomaly.

In some embodiments, the subject is one who undergoes a heart surgery after having been administered a first dose of nirsevimab before or during their first RSV season, and the subject is administered a second dose of nirsevimab after the surgery. In some embodiments, the heart surgery is cardiac surgery with cardiopulmonary bypass. In some embodiments, the subject is administered the second dose of nirsevimab within 90 days of having been administered the first dose of nirsevimab, and the second dose of nirsevimab is 50 mg if the subject is <5 kg at the time of administration of the second dose and is 100 mg if the subject is ≥5 kg at the time of administration of the second dose. In some embodiments, the subject is administered the second dose of nirsevimab more than 90 days after receiving the first dose of nirsevimab, and the second dose of nirsevimab is 50 mg (regardless of the subject's weight). In some embodiments, the subject is administered the second dose of nirsevimab as soon as the subject is stable after surgery.

In some embodiments, the subject is one who undergoes a heart surgery after having been administered a first dose of nirsevimab before or during their second RSV season, and the subject is administered a second dose of nirsevimab after the surgery. In some embodiments, the heart surgery is cardiac surgery with cardiopulmonary bypass. In some embodiments, the subject is administered the second dose of nirsevimab within 90 days of having been administered the first dose of nirsevimab, and the second dose of nirsevimab is 200 mg. In some embodiments, the subject is administered the second dose of nirsevimab more than 90 days after receiving the first dose of nirsevimab, and the second dose of nirsevimab is 100 mg. In some embodiments, the subject is administered the second dose of nirsevimab as soon as the subject is stable after surgery.

RSV infections can be diagnosed and monitored by a variety of known tests. These tests may be performed on upper and lower respiratory specimens and include, for example, real-time reverse transcriptase-polymerase chain reaction (rRT-PCR), antigen testing, viral culture, serology. Some tests can differentiate between RSV subtypes (A and B).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein.

In order that this invention may be better understood, the following representative embodiments and examples are set forth. These representative embodiments and examples are for purposes of illustration only and are not to be construed as limiting the scope of the invention in any manner.

### Representative Embodiments of the Disclosure

1. A method of preventing a respiratory syncytial virus (RSV) lower respiratory tract infection (LRTI) in an infant or pediatric subject in need thereof, comprising administering to the subject a single dose of nirsevimab at a timepoint that is outside the RSV season, wherein the administration occurs not more than once per year.

2. The method of embodiment 1, 91, or 94, wherein the subject is administered nirsevimab intramuscularly.

3. The method of embodiment 1, 91, or 94, wherein the subject is administered nirsevimab subcutaneously.

4. The method of any one of embodiments 1-3 or 91, wherein the subject is an infant born outside the RSV season and the timepoint of nirsevimab administration is from birth to a time that is before the start of the infant's first RSV season.

5. The method of any one of embodiments 1˜4 or 91, wherein the RSV season is about five months long.

6. The method of any one of embodiments 1˜4 or 91, wherein the RSV season is longer than about five months.

7. The method of any one of embodiments 1-6 or 91, wherein the subject is administered a single dose of nirsevimab about two weeks before the beginning of the RSV season.

8. The method of any one of embodiments 1-6 or 91, wherein the subject is administered a single dose of nirsevimab about three weeks before the beginning of the RSV season.

9. The method of any one of embodiments 1-6 or 91, wherein the subject is administered a single dose of nirsevimab about four weeks before the beginning of the RSV season.

10. The method of any one of embodiments 1-6 or 91, wherein the subject is administered a single dose of nirsevimab about 1 month before the beginning of the RSV season.

11. The method of any one of embodiments 1-6 or 91, wherein the subject is administered a single dose of nirsevimab about 2 months before the beginning of the RSV season.

12. The method of any one of embodiments 1-6 or 91, wherein the subject is administered a single dose of nirsevimab about 3 months before the beginning of the RSV season.

13. The method of any one of embodiments 1-6 or 94, wherein the subject is administered a single dose of nirsevimab about one week following the end of the last RSV season and wherein the last RSV season is longer than about nine months.

14. The method of any one of embodiments 1-6 or 94, wherein the subject is administered a single dose of nirsevimab about two weeks following the end of the last RSV season and wherein the last RSV season is longer than about nine months.

15. The method of any one of embodiments 1-6 or 94, wherein the subject is administered a single dose of nirsevimab about three weeks following the end of the last RSV season and wherein the last RSV season is longer than about nine months.

16. The method of any one of embodiments 1-6 or 94, wherein the subject is administered a single dose of nirsevimab about four weeks following the end of the last RSV season and wherein the last RSV season is longer than about eight months.

17. The method of any one of embodiments 1-6 or 94, wherein the subject is administered a single dose of nirsevimab about 1 month following the end of the last RSV season and wherein the last RSV season is longer than about eight months.

18. The method of any one of embodiments 1-6 or 94, wherein the subject is administered a single dose of nirsevimab about 2 months following the end of the last RSV season and wherein the last RSV season is longer than about seven months.

19. The method of any one of embodiments 1-6 or 94, wherein the subject is administered a single dose of nirsevimab about 3 months following the end of the last RSV season and wherein the last RSV season is longer than about six months.

20. The method of any one of embodiments 1-6 or 94, wherein the subject is administered a single dose of nirsevimab about 4 months following the end of the last RSV season and wherein the last RSV season is longer than about five months.

21. The method of any one of embodiments 1-6 or 94, wherein the subject is administered a single dose of nirsevimab about 5 months following the end of the last RSV season and wherein the last RSV season is longer than about four months.

22. The method of any one of embodiments 1-6 or 94, wherein the subject is administered a single dose of nirsevimab about 6 months following the end of the last RSV season and wherein the last RSV season is longer than about three months.

23. The method of any one of embodiments 1-6 or 94, wherein the subject is administered a single dose of nirsevimab about 7 months following the end of the last RSV season and wherein the last RSV season is longer than about two months.

24. The method of any one of embodiments 1-12 or 91, wherein the beginning of the RSV season is defined by the first two consecutive weeks during which the average percentage of positive RSV tests over the two week period exceeds a threshold, wherein the threshold is from 3%-13%.

25 The method of embodiment 24, wherein the threshold is 3%.

26. The method of embodiment 24, wherein the threshold is 5%.

27. The method of embodiment 24, wherein the threshold is 7%,

28. The method of embodiment 24, wherein the threshold is 10%.

29. The method of embodiment 24, wherein the threshold is 13%.

30. The method of any one of embodiments 13-23 or 94, wherein the end of the last RSV season is defined by the first week during which the average percentage of positive RSV tests over the one week period is below a threshold, wherein the threshold is from 3%-13%.

31. The method of embodiment 30, wherein the threshold is 3%.

32. The method of embodiment 30, wherein the threshold is 5%.

33. The method of embodiment 30, wherein the threshold is 7%.

34. The method of embodiment 30, wherein the threshold is 10%.

35. The method of embodiment 30, wherein the threshold is 13%.

36. The method of any one of embodiments 24-35, wherein the test is a PCR test.

37. The method of any one of embodiments 24-35, wherein the test is an antigen test.

38. The method of any one of embodiments 1-12 or 91, wherein the beginning of the RSV season is defined by the first two consecutive weeks during which the average percentage of positive PCR RSV tests exceeds 3%.

39. The method of any one of embodiments 1-12 or 91, wherein the beginning of the RSV season is defined by the first two consecutive weeks during which the average percentage of positive antigen tests exceeds 10%.

40. The method of any one of embodiments 13-23 or 94, wherein the end of the last RSV season is defined by the first week during which the average percentage of positive PCR-tests is below 3%.

41. The method of any one of embodiments 13-23 or 94, wherein the end of the last RSV season is defined by the first week during which the average percentage of positive antigen tests is below 10%.

42. The method of any one of embodiments 1-12 or 91, wherein the beginning of the RSV season is determined by a meteorological season, wherein the meteorological season is fall (autumn).

43. The method of any one of embodiments 1-12 or 91, wherein the beginning of the RSV season is predicted by a local, state, regional, or national health authority.

44. The method of any one of embodiments 13-23 or 94, wherein the end of the RSV season is declared by a local, state, regional, or national health authority.

45. A method of preventing a respiratory syncytial virus (RSV) lower respiratory tract infection (LRTI) in an infant or pediatric subject in need thereof, comprising intramuscularly administering to the subject a single dose of nirsevimab per RSV season before the beginning of the RSV season, at the beginning of the RSV season or during the RSV season, wherein the RSV season is longer than about five months, optionally wherein the RSV season is about six, about seven, about eight, about nine, or about ten months long.

46. A method of preventing a respiratory syncytial virus (RSV) lower respiratory tract infection (LRTI) in an infant or pediatric subject in need thereof, comprising intramuscularly administering to the subject a single dose of nirsevimab, wherein the single dose provides protection against RSV infection and/or against RSV disease for more than five months, preferably for at least six, seven, eight, nine, ten, eleven, or twelve months.

47. The method of any one of embodiments 1-46, 91, or 94, wherein the subject is a healthy late preterm or term infant born ≥35 weeks 0 days gestational age (GA) and is one year of age or younger.

48. The method of embodiment 47, wherein the subject is <5 kg and receives a 50 mg dose of nirsevimab.

49. The method of embodiment 47, wherein the subject is ≥5 kg and receives a 100 mg dose of nirsevimab.

50. The method of any one of embodiments 1-46, 91, or 94, wherein the subject is a healthy preterm infant born with a gestational age of 29 weeks 0 days through 34 weeks 6 days and is one year of age or younger.

51. The method of embodiment 50, wherein the subject is <5 kg and receives a 50 mg dose of nirsevimab.

52. The method of embodiment 50, wherein the subject is ≥5 kg and receives a 100 mg dose of nirsevimab.

53. The method of any one of embodiments 1-46, 91, or 94, wherein the subject is at a high risk of developing RSV LRTI.

54. The method of any one of embodiments 1-46, 91, or 94, wherein the subject is at a high risk of developing an RSV infection.

55. The method of embodiments 53 or 54, wherein the subject is a pediatric subject.

56. The method of any one of embodiments 53-55, wherein the subject is born with a gestational age less than 29 weeks; and/or has chronic lung disease (CLD), congenital heart disease (CHD), a suppressed immune system, a compromised immune system, an immunodeficiency, a neuromuscular disorder, Down's syndrome, a congenital airway anomaly, and/or cystic fibrosis.

57. The method of embodiment 56, wherein the subject is <5 kg, and wherein the subject is one year of age or younger and receives a 50 mg dose of nirsevimab.

58. The method of embodiment 56, wherein the subject is ≥5 kg, and wherein the subject is one year of age or younger and receives a 100 mg dose of nirsevimab.

59. The method of embodiment 56, wherein the subject is ≥5 kg, and wherein the subject is entering their second or later year of life and receives a 200 mg dose of nirsevimab.

60. The method of embodiment 56, wherein the subject is a pediatric patient born with a gestational age less than 29 weeks; and/or has chronic lung disease or congenital heart disease.

61. The method of embodiment 60, wherein the subject is ≥5 kg, and wherein the subject is entering their second or later year of life and receives a 200 mg dose of nirsevimab.

62. The method of any one of embodiments 53-61, wherein the subject is a pediatric patient entering their first or second RSV season.

63. The method of any one of embodiments 1-62, 91, or 94, wherein the administration occurs before the subject's first RSV season.

64. A method of preventing a respiratory syncytial virus (RSV) lower respiratory tract infection (LRTI) in an infant or pediatric subject, comprising intramuscularly administering to the subject a single dose of nirsevimab, wherein the administration occurs not more than once per year.

65. A method of preventing a respiratory syncytial virus (RSV) lower respiratory tract infection (LRTI) in an infant or pediatric subject in need thereof, comprising intramuscularly administering to the subject a single dose of nirsevimab, wherein the administration occurs not more than once per season.

66. The method of embodiment 64 or 65, wherein administration occurs outside of the RSV season.

67. The method of embodiment 64 or 65, wherein administration occurs during the RSV season.

68. A method of obtaining protection from a respiratory syncytial virus (RSV) lower respiratory tract infection (LRTI) and/or RSV disease in an infant or pediatric subject, comprising intramuscularly administering to the subject a single dose of nirsevimab, wherein the single dose provides protection for more than five to at least eight months, optionally about 12 months.

69. A method of obtaining protection from a respiratory syncytial virus (RSV) lower respiratory tract infection (LRTI) and/or RSV disease in an infant or pediatric subject, comprising intramuscularly administering to the subject a single dose of nirsevimab, wherein the single dose provides protection for at least one RSV season.

70. The method of any one of embodiments 1-69, 91, or 94, wherein the single dose contains 50-200 mg nirsevimab.

71. The method of any one of embodiments 1-70, 91, or 94, wherein the amount of nirsevimab in the single dose is
50 mg if the subject is an infant weighing <5 kg at the time of administration,
100 mg if the subject is an infant weighing ≥5 kg at the time of administration, or
200 mg if the subject is a pediatric subject entering their second RSV season.

72. The method of embodiment 71, wherein the pediatric subject entering their second RSV season is at a high risk of RSV infection.

73. The method of any one of embodiments 1-72, 91, or 94, where preventing RSV LRTI comprises preventing medically attended RSV LRTI, optionally bronchiolitis or pneumonia.

74. The method of any one of embodiments 1-73, 91, or 94, wherein preventing RSV LRTI comprises preventing RSV-associated hospitalization.

75. The method of any one of embodiments 1-73, 91, or 94, wherein preventing RSV LRTI comprises preventing severe RSV LRTI.

76. The method of any one of embodiments 1-73, 91, or 94, wherein preventing RSV LRTI comprises preventing very severe RSV LRTI.

77. The method of any one of embodiments 1-73, 91, or 94, wherein preventing RSV LRTI comprises preventing all-cause LRTI.

78. The method of any one of embodiments 1-77, 91, or 94, wherein preventing RSV LRTI comprises preventing all-cause LRTI hospitalization.

79. A method of preventing very severe respiratory syncytial virus (RSV) infection in an infant or pediatric subject, comprising administering to the subject a single dose of nirsevimab, wherein the single dose is effective to prevent very severe RSV infection for more than five months.

80. A method of preventing all-cause lower respiratory tract infection (LRTI) or all-cause LRTI hospitalization in an infant or pediatric subject, comprising administering to the subject a single dose of nirsevimab, wherein the single dose is effective to prevent all-cause LRTI or all-cause hospitalization for more than five months.

81. A method of preventing very severe RSV LRTI in an infant or pediatric subject in need thereof, comprising administering to the subject a single dose of nirsevimab at a timepoint that is outside the RSV season, wherein the administration occurs not more than once per year.

82. The method of any one of embodiments 1-81, 91, or 94, wherein the single dose of nirsevimab is provided in a pharmaceutical composition comprising:
100 mg/mL nirsevimab,
30 mM histidine/histidine-HCl,
80 mM arginine-HCl,
120 mM sucrose, and
0.02%-0.04% (w/v) polysorbate 80, pH 6.0.

83. The method of any one of embodiments 1-82, 91, or 94, wherein the amount of nirsevimab in the single dose is effective to yield serum AUC_{0-∞}» of greater than 12.8 day mg/mL in the subject.

84. The method of any one of embodiments 1-83, 91, or 94, wherein the amount of nirsevimab in the single dose is effective to yield elevated RSV neutralizing antibody (Nab) levels (e.g., >5-fold higher, >7-fold higher) in the subject at about 12 months after administration compared to the subject's RSV Nab levels at the time of administration.

85. The method of any one of embodiments 1-84, 91, or 94, wherein the amount of nirsevimab in the single dose is effective to yield elevated RSV Nab levels (e.g., >3-fold higher) in the subject at about 12 months after administration compared to RSV Nab levels of a subject with a confirmed RSV infection and who was not administered nirsevimab.

86. The method of any one of embodiments 1-85, 91, or 94, wherein the amount of nirsevimab in the single dose is effective to reduce the subject's antibiotic use compared to the antibiotic use of a subject who was not administered nirsevimab.

87. The method of any one of claim 1-86, 91, or 94, wherein the single dose of nirsevimab is administered intramuscularly or subcutaneously.

88. A method of preventing RSV LRTI in a subject at high risk of developing an RSV infection, comprising administering to the subject a first dose of nirsevimab before the subject's first RSV season and a second dose of nirsevimab before the subject's second RSV season, wherein the amount of nirsevimab in the first dose is
50 mg if the subject weighs <5 kg at the time of administration, and
100 mg if the subject weighs ≥5 kg at the time of administration;
   and wherein the amount of nirsevimab in the second dose is 200 mg.

89. A method of preventing RSV LRTI in a subject undergoing a heart surgery, wherein the method comprises:
a) administering to the subject a first dose of nirsevimab before the subject's first RSV season, wherein
   i) the amount of nirsevimab in the first dose is 50 mg if the subject weighs <5 kg at the time of administration of the first dose; or
   ii) the amount of nirsevimab in the first dose is 100 mg if the subject weighs ≥5 kg at the time of administration of the first dose; and
b) administering to the subject a second dose of nirsevimab after the heart surgery, wherein
   i) the amount of nirsevimab in the second dose is 50 mg if the subject weighs <5 kg at the time of administration of the second dose and the second dose is administered within 90 days of administration of the first dose; or
   ii) the amount of nirsevimab in the second dose is 100 mg if the subject weighs ≥5 kg at the time of administration of the second dose and the second dose is administered within 90 days of administration of the first dose; or
   iii) the amount of nirsevimab in the second dose is 50 mg if the second dose is administered more than 90 days after administration of the first dose,
optionally wherein the second dose is administered as soon as the subject is stable after the surgery, optionally wherein the heart surgery is a cardiac surgery with cardiopulmonary bypass.

90. The method of any one of embodiments 1-89, 91, or 94, wherein a dose of nirsevimab is administered at birth.

91. A method of preventing respiratory syncytial virus (RSV) lower respiratory tract infection (LRTI) in an infant or pediatric subject, comprising administering to the subject a single dose of nirsevimab before the beginning of the RSV season, wherein the single dose is effective to prevent RSV LRTI for more than five months.

92. The method of embodiment 91, wherein the subject is administered the single dose of nirsevimab about two weeks, about three weeks, about four weeks, about one month, about two months, or about three months before the beginning of the RSV season.

93. The method of embodiment 91 or 92, wherein the beginning of the RSV season is defined by the first two consecutive weeks during which the average percentage of positive RSV tests over the two-week period exceeds a threshold, wherein the threshold is from 3%-13%, optionally wherein (i) the RSV tests are PCR tests and the threshold is 3% or (ii) the RSV tests are antigen tests and the threshold is 10%.

94. A method of preventing respiratory syncytial virus (RSV) lower respiratory tract infection (LRTI) in an infant or pediatric subject, comprising administering to the subject a single dose of nirsevimab following the end of the last RSV season, wherein the single dose is effective to prevent RSV LRTI throughout the next RSV season.

95. The method of embodiment 94, wherein the subject is administered the single dose of nirsevimab about one week, about two weeks, about three weeks, about four weeks, about one month, about two months, about three months, about four months, about five months, about six months, or about seven months following the end of the last RSV season.

96. The method of embodiment 94 or 95, wherein the end of the last RSV season is defined by the first week during which the average percentage of positive RSV tests over the one-week period is below a threshold, wherein the threshold is from 3%-13%, optionally wherein (i) the RSV tests are PCR tests and the threshold is 3% or (ii) the RSV tests are antigen tests and the threshold is 10%.

97. A pharmaceutical composition comprising nirsevimab for use in the method of any one of embodiments 1-96.

98. Use of nirsevimab in the manufacture of a medicament for use in the method of any one of embodiments 1-96.

99. An article of manufacture comprising nirsevimab for use in the method of any one of embodiments 1-96.

100. The article of manufacture of embodiment 99, comprising a single-unit container or a multi-unit container, wherein each unit is about 50, 100, or 200 mg of nirsevimab.

101. The article of manufacture of embodiment 99 or 100, wherein the container is a vial or a pre-filled syringe or injector.

102. A pharmaceutical composition comprising a single dose of nirsevimab for use in preventing respiratory syncytial virus (RSV) lower respiratory tract infection (LRTI) in an infant or pediatric subject, wherein the pharmaceutical composition is to be administered to the subject before the beginning of the RSV season and is effective to prevent RSV LRTI for more than five months.

103. The pharmaceutical composition of embodiment 102, wherein the pharmaceutical composition is to be administered to the subject about two weeks, about three weeks, about four weeks, about one month, about two months, or about three months before the beginning of the RSV season.

104. The pharmaceutical composition of embodiment 102 or 103, wherein the beginning of the RSV season is defined by the first two consecutive weeks during which the average percentage of positive RSV tests over the two-week period exceeds a threshold, wherein the threshold is from 3%-13%, optionally wherein (i) the RSV tests are PCR tests and the threshold is 3% or (ii) the RSV tests are antigen tests and the threshold is 10%.

105. A pharmaceutical composition comprising a single dose of nirsevimab for use in preventing respiratory syncytial virus (RSV) lower respiratory tract infection (LRTI) in an infant or pediatric subject, wherein the pharmaceutical composition is to be administered to the subject following the end of the last RSV season and is effective to prevent RSV LRTI throughout the next RSV season.

106. The pharmaceutical composition of embodiment 105, wherein the pharmaceutical composition is to be administered to the subject about one week, about two weeks, about three weeks, about four weeks, about one month, about two months, about three months, about four months, about five months, about six months, or about seven months following the end of the last RSV season.

107. The pharmaceutical composition of embodiment 105 or 106, wherein the end of the last RSV season is defined by the first week during which the average percentage of positive RSV tests over the one-week period is below a threshold, wherein the threshold is from 3%-13%, optionally wherein (i) the RSV tests are PCR tests and the threshold is 3% or (ii) the RSV tests are antigen tests and the threshold is 10%.

108. The pharmaceutical composition of any one of embodiments 102-107, wherein the subject is at a high risk of developing an RSV infection.

109. The pharmaceutical composition of embodiment 108, wherein the subject is born with a gestational age less than 29 weeks; and/or has chronic lung disease (CLD), congenital heart disease (CHD), a suppressed immune system, a compromised immune system, an immunodeficiency, a neuromuscular disorder, Down's syndrome, a congenital airway anomaly, and/or cystic fibrosis.

110. A pharmaceutical composition comprising a single dose of nirsevimab for use in preventing very severe respiratory syncytial virus (RSV) infection in an infant or pediatric subject, wherein the pharmaceutical composition is effective to prevent very severe RSV infection for more than five months.

111. A pharmaceutical composition comprising a single dose of nirsevimab for use in preventing all-cause lower respiratory tract infection (LRTI) or all-cause LRTI hospitalization in an infant or pediatric subject, wherein the pharmaceutical composition is effective to prevent all-cause LRTI or all-cause hospitalization for more than five months.

112. Use of nirsevimab in the manufacture of a medicament for preventing respiratory syncytial virus (RSV) lower respiratory tract infection (LRTI) in an infant or pediatric subject, wherein the medicament is to be administered to the subject in a single dose before the beginning of the RSV season and is effective to prevent RSV LRTI for more than five months.

113. The use of embodiment 112, wherein the medicament is to be administered to the subject about two weeks, about three weeks, about four weeks, about one month, about two months, or about three months before the beginning of the RSV season.

114. The use of embodiment 112 or 113, wherein the beginning of the RSV season is defined by the first two consecutive weeks during which the average percentage of positive RSV tests over the two-week period exceeds a threshold, wherein the threshold is from 3%-13%, optionally wherein (i) the RSV tests are PCR tests and the threshold is 3% or (ii) the RSV tests are antigen tests and the threshold is 10%.

115. Use of nirsevimab in the manufacture of a medicament for preventing respiratory syncytial virus (RSV) lower respiratory tract infection (LRTI) in an infant or pediatric subject, wherein the medicament is to be administered to the subject in a single dose following the end of the last RSV season and is effective to prevent RSV LRTI throughout the next RSV season.

116. The use of embodiment 115, wherein the medicament is to be administered to the subject about one week, about two weeks, about three weeks, about four weeks, about one month, about two months, about three months, about four months, about five months, about six months, or about seven months following the end of the last RSV season.

117. The use of embodiment 115 or 116, wherein the end of the last RSV season is defined by the first week during which the average percentage of positive RSV tests over the one-week period is below a threshold, wherein the threshold is from 3%-13%, optionally wherein (i) the RSV tests are PCR tests and the threshold is 3% or (ii) the RSV tests are antigen tests and the threshold is 10%.

118. The use of any one of embodiments 112-117, wherein the subject is at a high risk of developing an RSV infection.

119. The pharmaceutical composition of embodiment 118, wherein the subject is born with a gestational age less than 29 weeks; and/or has chronic lung disease (CLD), congenital heart disease (CHD), a suppressed immune system, a compromised immune system, an immunodeficiency, a neuromuscular disorder, Down's syndrome, a congenital airway anomaly and/or cystic fibrosis.

120. Use of nirsevimab in the manufacture of a medicament for preventing very severe respiratory syncytial virus (RSV) infection in an infant or pediatric subject, wherein the medicament is to be administered to the subject in a single dose and is effective to prevent very severe RSV infection for more than five months.

121. Use of nirsevimab in the manufacture of a medicament for preventing all-cause lower respiratory tract infection (LRTI) or all-cause LRTI hospitalization in an infant or pediatric subject, wherein the medicament is to be administered to the subject in a single dose and is effective to prevent all-cause LRTI or all-cause hospitalization for more than five months.

122. An article of manufacture comprising a single dose of nirsevimab for use in preventing respiratory syncytial virus (RSV) lower respiratory tract infection (LRTI) in an infant or pediatric subject, wherein the single dose is to be administered to the subject before the beginning of the RSV season and is effective to prevent RSV LRTI for more than five months.

123. The article of manufacture of embodiment 122, wherein the single dose is to be administered to the subject about two weeks, about three weeks, about four weeks, about one month, about two months, or about three months before the beginning of the RSV season.

124. The article of manufacture of embodiment 122 or 123, wherein the beginning of the RSV season is defined by the first two consecutive weeks during which the average percentage of positive RSV tests over the two-week period exceeds a threshold, wherein the threshold is from 3%-13%, optionally wherein (i) the RSV tests are PCR tests and the threshold is 3% or (ii) the RSV tests are antigen tests and the threshold is 10%.

125. An article of manufacture comprising a single dose of nirsevimab for use in preventing respiratory syncytial virus (RSV) lower respiratory tract infection (LRTI) in an infant or pediatric subject, wherein the single dose is to be administered to the subject following the end of the last RSV season and is effective to prevent RSV LRTI throughout the next RSV season.

126. The article of manufacture of embodiment 125, wherein the single dose is to be administered to the subject about one week, about two weeks, about three weeks, about four weeks, about one month, about two months, about three months, about four months, about five months, about six months, or about seven months following the end of the last RSV season.

127. The article of manufacture of embodiment 125 or 126, wherein the end of the last RSV season is defined by the first week during which the average percentage of positive RSV tests over the one-week period is below a threshold, wherein the threshold is from 3%-13%, optionally wherein (i) the RSV tests are PCR tests and the threshold is 3% or (ii) the RSV tests are antigen tests and the threshold is 10%.

128. The article of manufacture of any one of embodiments 122-127, wherein the subject is at a high risk of developing an RSV infection.

129. The article of manufacture of embodiment 128, wherein the subject is born with a gestational age less than 29 weeks; and/or has chronic lung disease (CLD), congenital heart disease (CHD), a suppressed immune system, a compromised immune system, an immunodeficiency, a neuromuscular disorder, Down's syndrome, a congenital airway anomaly, and/or cystic fibrosis.

130. An article of manufacture comprising a single dose of nirsevimab for use in preventing very severe respiratory syncytial virus (RSV) infection in an infant or pediatric subject, wherein the single dose is effective to prevent very severe RSV infection for more than five months.

131. An article of manufacture comprising a single dose of nirsevimab for use in preventing all-cause lower respiratory tract infection (LRTI) or all-cause LRTI hospitalization in an infant or pediatric subject, wherein the single dose is effective to prevent all-cause LRTI or all-cause hospitalization for more than five months.

### EXAMPLES

### Example 1: Safety and Efficacy of Nirsevimab Against Respiratory Syncytial Virus in Healthy Late Preterm and Term Infants

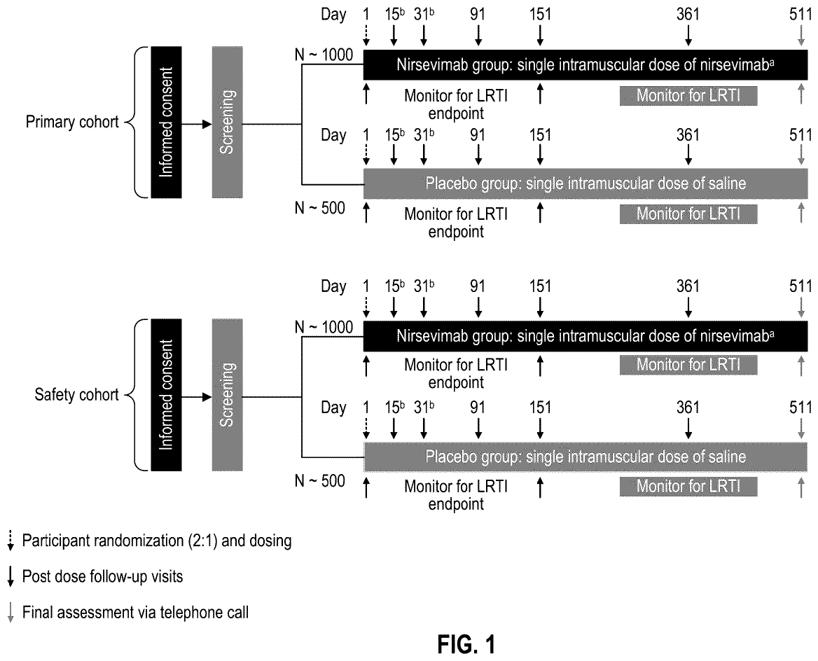

This Example describes a clinical trial protocol for a Phase 3, randomized, double-blind, placebo-controlled study to evaluate the safety and efficacy of nirsevimab against RSV in healthy late preterm and term infants. This is a single-dose study to determine if nirsevimab will prevent medically attended RSV-confirmed LRTI in healthy infants entering their first RSV season (FIG. 1).

### Primary Objective and Associated Endpoint

The primary objective is to assess the efficacy of nirsevimab when administered as a single fixed intramuscular (IM) dose to infants ≥35 weeks 0 days GA and entering their first RSV season, in reducing medically attended LRTI due to reverse-transcriptase-polymerase chain reaction (RT-PCR)-confirmed RSV, compared to placebo through 150 days after administration. The endpoint for primary efficacy is the incidence of medically attended LRTI (inpatient and outpatient) due to RT-PCR confirmed RSV through 150 days after dosing (i.e., during a 5-month RSV season). After RSV detection by RT-PCR, RSV A, and RSV B subtypes will be determined by genotypic analysis of sequence changes in the mature F protein from all RSV-positive isolates/samples compared with contemporary RSV A and RSV B reference strains. The case definition of medically attended RSV LRTI for the primary endpoint is provided in Table 1 below (one item from each column is required to meet the case definition of RSV LRTI). Subgroup analyses of the primary endpoint were prespecified according to hemisphere, age at randomization, sex, race, weight, and gestational age.

TABLE 1
Elements to Evaluate for Case Definition of RSV LRTI

| | | Medical Significance |
|---|---|---|
| | | Objective Measures of Clinical |
| RSV | Lower Respiratory Tract | Severity (must have ≥1) |
| RSV PCR | ≥1 Documented physical | Increased respiratory rate^{a} |
| positive | examination findings | |
| | localizing to LRT: | |
| | Rhonchi | |
| | Rales | |
| | Crackles | |
| | Wheeze | |
| | | Hypoxemia in room air: |
| | | O₂ <95% at ≤1800 m |
| | | O₂ <92% at >1800 m |
| | | Clinical signs of severe respiratory |
| | | disease: |
| | | New-onset apnea |
| | | Retractions |
| | | Grunting |
| | | Nasal flaring |
| | | Acute hypoxic or ventilatory |
| | | failure |
| | | Dehydration due to respiratory |
| | | distress requiring IV hydration |
| | | Intercostal, subcostal, or |
| | | supraventricular retractions |

^{a}≥60 breaths/min for <2-month-old; ≥50 breaths/min for 2-6-month old, ≥40 breaths/min for 6-24-month old. LRT, lower respiratory tract; IV, intravenous; PCR, polymerase chain reaction. See Villafana et al., Expert Rev Vac. (2017) 16: 1-13.

### Secondary Objectives and Endpoints

### Efficacy

A secondary objective is to assess the efficacy of nirsevimab in reducing hospitalizations due to RT-PCR-confirmed RSV, compared to placebo. The associated endpoint for secondary efficacy is the incidence of hospitalization due to this condition during the same period. All cases were confirmed by central laboratory testing of RSV using real-time RT-PCR.

### Safety

Another secondary objective is to evaluate the safety and tolerability of nirsevimab when administered as a single fixed IM dose, compared to placebo. The associated endpoint is safety and tolerability of nirsevimab as assessed by the occurrence of treatment-emergent adverse events (TEAEs), treatment-emergent serious adverse events (TESAEs), adverse event of special interest (AESIs), and new onset chronic diseases (NOCDs).

### Pharmacokinetics (PK)

Another secondary objective is to evaluate single-dose serum concentrations of nirsevimab. The associated endpoint is the summary of nirsevimab serum concentrations and estimated PK parameters (apparent clearance and AUC_{0-∞}s). The pharmacokinetics of nirsevimab were determined as described previously (Domachowske et al., Pediatr Infect Dis J. (2018) 37:886-92).

### Anti-drug Antibody

Another secondary objective is to evaluate anti-drug antibody (ADA) responses to nirsevimab in serum. The associated endpoint is the incidence of ADA to nirsevimab in serum. A positive anti-nirsevimab antibody response was defined as a titer of ≥50, as previously described (Griffin et al., N Engl J Med. (2020) 383:415-25; Domachowske, supra).

### Exploratory Objectives and Endpoints

### Healthcare Resource Utilization and Caregiver Burden

An exploratory objective is to assess healthcare resource utilization and caregiver burden for nirsevimab recipients compared to placebo recipients. The associated endpoints are:
Magnitude of healthcare resource utilization: e.g., number of admissions to hospitals and intensive care units and duration of stay; number of subjects who require respiratory support and supplemental oxygen and the duration of use; number and type of outpatient visits (e.g., emergency room, urgent care, outpatient clinic); and number of prescription and over-the-counter medications and duration of use; and
Caregiver burden for subjects with medically attended LRTI caused by RT-PCR-confirmed RSV: caregiver missed work days; and subject absence from day care.

### RSV Neutralizing Antibody

Another exploratory objective is to determine anti-RSV neutralizing antibody levels in serum afforded by a single dose of nirsevimab compared to maternal RSV neutralizing antibody levels and those elicited following infection in the placebo group. The associated endpoint is anti-RSV neutralizing antibody levels (IU/mL) in serum for nirsevimab recipients compared to placebo recipients.

### RSV Serology

Another exploratory objective is to evaluate exposure to RSV by measuring sero-responses to different RSV proteins. The associated endpoints are:
Antibody levels to RSV pre-F, post-F, Ga, Gb, and N at different time points; and
Changes in antibody levels (sero-response) indicating exposure to RSV.

### RSV Resistance Monitoring

Another exploratory objective is to characterize resistance to nirsevimab through genotypic and phenotypic analyses. The associated endpoint is genotypic analysis and susceptibility of RSV variants to neutralization by nirsevimab.

RSV LRTI after Day 151

Another exploratory objective is to assess the incidence of medically attended LRTI due to RT-PCR-confirmed RSV, compared to placebo after Day 151. The associated endpoint is incidence of medically attended LRTI (inpatient and outpatient) due to RT-PCR-confirmed RSV from Day 152 to Day 361.

### Study Design

This study is to determine if nirsevimab will prevent medically attended RSV-confirmed LRTI in healthy infants entering their first RSV season. The population to be enrolled is healthy late preterm and term infants born ≥35 weeks 0 days GA entering their first RSV season who would not receive RSV prophylaxis based on the American Academy of Pediatrics (AAP) or other local or national guidelines. Briefly, a total of approximately 3,000 infants will be enrolled. The subjects will be randomly assigned in a 2:1 ratio to receive a single IM dose of nirsevimab (N=2,000) or placebo (N=1,000). The nirsevimab dose level will be stratified by body weight at time of dosing: 50 mg nirsevimab for infants <5 kg of body weight or 100 mg nirsevimab for infants ≥5 kg of body weight. Subjects in the placebo group will receive a corresponding volume of normal saline, i.e., 0.5 mL if body weight <5 kg or 1.0 mL if body weight ≥5 kg. Randomization will be stratified by hemisphere (northern hemisphere (NH), southern hemisphere (SH)) and by subject age at the time of randomization (≤3 months, >3 to ≤6 months, >6 months). Enrollment of infants >6 months of age will be limited to approximately 500.

The study will comprise 2 cohorts: a primary cohort (N=˜ 1,500) and a complementary safety cohort (hereafter referred to as the safety cohort; N=˜ 1,500) for a total of approximately 3,000 subjects. The primary cohort included participants enrolled from 2019 across 150 sites (20 countries) in the NH and from 2020 across 10 sites (1 country) in the SH. The safety cohort will include subjects enrolled after the NH2020 enrollment season. Given the largely reduced circulation of RSV due to the coronavirus disease 2019 (COVID-19) pandemic related measures, the efficacy analyses performed in the primary analysis for the primary cohort will serve the purpose of evaluating the efficacy of nirsevimab.

All subjects will be followed for approximately 510 days after dosing. Subjects will be monitored throughout the study for LRTI. All subjects seeking medical attention for a respiratory illness (in either the inpatient or outpatient setting) will be evaluated for the occurrence of LRTI. All subjects found to have an LRTI and all subjects who require hospitalization for a respiratory infection, even if there is not a diagnosis of LRTI, should have respiratory samples obtained and respiratory assessment forms completed. Samples should be collected for all of these events (even those not meeting the protocol definition of LRTI). Subjects who have a primary hospitalization for a respiratory infection (i.e., upper or lower tract) or a respiratory deterioration during a hospitalization, or who seek outpatient medical attention (including ER visits) for a lower respiratory illness, will be assessed clinically for the presence of LRTI and for RSV by central laboratory diagnostic testing of respiratory secretions.

In addition to the clinical assessment of LRTI, there is a protocol definition using objective criteria for the determination of a medically attended protocol-defined LRTI (Table 1).

Testing for RSV will be performed centrally using the United States Food and Drug Administration-cleared and European Conformity-marked in vitro diagnostic real-time RT-PCR assay (Lyra RSV+human metapneumovirus [hMPV] assay; Quidel Corporation, San Diego, CA). A diagnosis of RSV LRTI requires having a respiratory sample positive for RSV by the central laboratory RT-PCR.

The study will be conducted over 5 respiratory virus seasons (3 northern hemisphere (NH) and 2 southern hemisphere (SH) seasons) to better characterize RSV cases over multiple seasons.

Blood samples for pharmacokinetics (PK) and anti-drug antibody (ADA) will be collected at screening or Day 1 predose, on Days 31, 151, and 361, and from subjects hospitalized for a respiratory infection through Day 361. In Japan, the Day 15 visit will be replaced by visit on Day 8 for blood sample collection (laboratory parameters). Blood samples will also be collected at visits on Days 31 and 151 in Japan. In Europe, the Day 31 assessment for RSV serology will be removed to limit the amount of blood drawn. Safety assessments will be performed through Day 361. The dose level will be stratified by body weight at time of dosing.

### Inclusion Criteria

Subjects must meet all of the following criteria:
Healthy infants in their first year of life and born ≥35 weeks 0 days GA (infants who have an underlying illness such as cystic fibrosis or Down syndrome with no other risk factors are eligible);
Infants who are entering their first RSV season at the time of screening; and
The subject is available to complete the follow-up period, which will be 17 months after receipt of study drug.

### Exclusion Criteria

Any of the following would exclude the subject from participation in the study:
Meets national or other local criteria to receive commercial palivizumab;
Any fever (≥100.4° F. (≥38.0° C.), regardless of route) or acute illness within 7 days prior to randomization;
Any history of LRTI or active LRTI prior to, or at the time of, randomization;
Known history of RSV infection or active RSV infection prior to, or at the time of, randomization;
Any drug therapy (chronic or other) within 7 days prior to randomization or expected receipt during the study with the exception of: a) multivitamins and iron; and b) infrequent use of over-the-counter (OTC) medications for the systemic treatment of common childhood symptoms (e.g., pain relievers) that may be permitted according to the judgment of the investigator;
Any current or expected receipt of immunosuppressive agents including steroids (except for the use of topical steroids according to the judgment of the investigator);
History of receipt of blood, blood products, or immunoglobulin products, or expected receipt through the duration of the study;
Receipt of any investigational drug;
Known renal impairment;
Known hepatic dysfunction including known or suspected active or chronic hepatitis infection;
History of CLD/bronchopulmonary dysplasia;
Clinically significant congenital anomaly of the respiratory tract;
Chronic seizure or evolving or unstable neurologic disorder;
CHD, except for children with uncomplicated CHD (e.g., patent ductus arteriosus, small septal defect);
Prior history of a suspected or actual acute life-threatening event;
Known immunodeficiency, including human immunodeficiency virus (HIV);
Mother with HIV infection (unless the child has been proven to be not infected);
Any known allergy, including to immunoglobulin products, or history of allergic reaction;
Receipt of palivizumab or other RSV mAb or any RSV vaccine, including maternal RSV vaccination;
Receipt of any monoclonal or polyclonal antibody (for example, hepatitis B immune globulin, IV immunoglobulin);
Any condition that, in the opinion of the investigator, would interfere with evaluation of the investigational product or interpretation of subject safety or study results; or
Concurrent enrollment in another interventional study.

### Statistical Methods General Considerations

There will be 2 study cohorts: a primary cohort and a safety cohort. The primary cohort will include subjects from the NH2019, SH2020, and NH2020 enrollment seasons (enrollment was paused after one subject from NH2020 was enrolled due to the impact of the COVID-19 pandemic). The safety cohort will include subjects enrolled after the NH2020 enrollment season. The Intent-to-treat (ITT) Population is defined as all subjects who are randomized. Subjects will be included in the treatment group corresponding to their randomized treatment. All analyses, with the exception of safety, will be performed on the ITT Population unless otherwise specified. Subjects in the ITT Population and from the primary cohort will be ITT Population 1 (ITT1). Subjects in the ITT Population and from the safety cohort will be ITT Population 2 (ITT2).

The As-treated Population will include all subjects who are randomized and who receive any amount of investigational product. Subjects will be included in the treatment group corresponding to the treatment actually received. All safety analyses will be performed on the As-treated Population. Subjects in the As-treated Population and from the primary cohort will be As-treated Population 1 (AT1). Subjects in the As-treated Population and from the safety cohort will be As-treated Population 2 (AT2).

### Statistical Analyses

There are 3 planned analyses for this study: the primary analysis, safety analysis, and final analysis. Efficacy analyses will be performed in the intent-to-treat population (all randomized participants) and safety analyses will be based on the as-treated population (participants who received any investigational product). The sample size of 3000 participants was selected for safety database consideration and provided >99% power for the primary efficacy endpoint. A sample size of approximately 1,500 participants for the primary efficacy analysis has ≥99% power to detect 70% relative risk reduction, at a two-sided significance level of 0.05, under the assumption of an 8% event rate in the placebo group.

The primary analysis will be conducted after all randomized subjects (except for one subject enrolled in the NH2020 season) from the primary cohort have been followed through Day 361 and will be the primary analysis for which the study is designed to assess efficacy. For the primary analysis, all efficacy, pharmacokinetics (PK), ADA, and safety data collected for the primary cohort through at least Day 361 will be analyzed. The safety analysis will be conducted when all subjects from the safety cohort have been followed through Day 151. For the safety analysis, in addition to the analyses conducted during the primary analysis based on the primary cohort, all available efficacy, PK, ADA, RSV neutralizing antibody, RSV serology, and safety data collected for the safety cohort will be analyzed (only descriptive summaries will be provided for the efficacy data collected for the safety cohort). The final analysis will be conducted when all subjects have completed the last visit of the study (i.e., Day 511). Given the largely reduced circulation of RSV due to the COVID-19 pandemic related measures, the efficacy analyses performed in the primary analysis for the primary cohort will serve the purpose of evaluating the efficacy of nirsevimab in the study population.

Although efficacy data will also be collected for the safety cohort, only descriptive summaries will be provided and there is no intent to pool the efficacy data from the safety cohort with that from the primary cohort. Both the primary and the safety cohorts, individually and combined, will serve the purpose of evaluating the safety of nirsevimab.

For participants without an RSV LRTI who are not followed through 150 days postdose, their event status will be considered missing and imputed with the observed event rate in the placebo group, with repeated imputation.

### Primary Efficacy Analysis

The incidence of RSV LRTI (inpatient and outpatient) during 5 months of the RSV season will be based on RSV test results (performed centrally via RT-PCR) and objective clinical LRTI criteria and will be presented by treatment group. For subjects with multiple medically attended RSV LRTI events, only the first occurrence will be used in the primary analysis.

The primary efficacy analysis of the primary endpoint will be conducted on ITT1. RSV LRTI that occurs through 150 days post dose will contribute to the primary efficacy analysis. For subjects who do not have a medically attended RSV LRTI and are not followed through 150 days post dose, their event status will be imputed assuming the observed placebo RSV LRTI rate conditional on stratification factors using multiple imputation techniques and will be described in the SAP. A Poisson regression model with robust variance will be used as the primary efficacy analysis model to compare the incidence of medically attended RSV LRTI between nirsevimab and placebo, including treatment group, age at the time of randomization (i.e., ≤3 months, >3 to ≤6 months, >6 months), and dichotomous temperate hemispheres (NH and SH) as covariates. In addition, the 2-sided p-value and corresponding 2-sided 95% confidence interval (CI) on the relative risk will be provided from the model. RRR is defined as (1-Pn/Ps) where Pn is the incidence of RSV LRTI through 150 days post dose in the nirsevimab group and Ps is the incidence of RSV LRTI through 150 days post dose in the placebo group generated by the model. Statistical significance will be achieved if the 2-sided p-value is ≤0.05.

During blinded data review prior to database lock for the primary analysis, it was decided to drop the stratification factor hemisphere from the full model due to no incidence of medically attended RSV LRTI events through 150 days post dose for SH in the primary cohort, which would cause a known convergence or estimation issue. Similar consideration also applies to other analyses for the primary efficacy endpoint, where hemisphere will be dropped from the corresponding models.

### Additional Analyses of the Primary Endpoint

A Cochran-Mantel-Haenszel approach stratified by age group at the time of randomization (i.e., ≤3 months, >3 to ≤6 months, >6 months) will be used to compare the incidence of RSV LRTI through 150 days post dose between treatment groups as a secondary analysis for the primary endpoint. The additional analyses will be conducted on ITT1. In addition, a time-to-event analysis assessing time to first RSV LRTI may be performed as a supplementary analysis. An analysis may also include all RSV positive LRTI endpoints, using results from either the central laboratory or local laboratory. Different approaches to handle missing data (i.e., early discontinuation and no RSV LRTI prior to discontinuation) may be considered for supplementary analyses. Additional analyses may be performed to adjust duration of efficacy follow-up and to assess the efficacy within subgroups. These analyses will be described in the SAP. The incidence of medically attended RSV LRTI through 150 days post dose will also be summarized by treatment group on ITT2.

### Secondary Endpoint Analyses

For efficacy analysis, the incidence of RSV LRTI hospitalization through 150 days post dose will be presented by treatment group. Similar methods as described above for the primary efficacy endpoint will be used to assess efficacy on RSV LRTI hospitalization on ITT1. The incidence of RSV LRTI hospitalization through 150 days post dose will also be summarized by treatment group on ITT2.

As described above, analysis of the primary endpoint will be performed using a Poisson regression model with robust variance. To control for the overall type I error, a hierarchical approach will be used. The secondary endpoint will be tested only if statistical significance for the primary endpoint was shown. That is, the secondary hypothesis will be tested at a significance level of 0.05 only if the treatment effect on the primary efficacy endpoint is demonstrated at the significance level of 2-sided 0.05. With that, the overall Type I error is controlled at 0.05. Therefore, no further multiplicity adjustment is necessary.

For safety analyses, they will be conducted on the overall As-treated Population, AT1, and AT2. Safety of nirsevimab will primarily be assessed by the occurrence of TEAEs and TESAEs. Adverse events will be graded according to the current version of the National Cancer Institute Common Terminology Criteria for Adverse Events where applicable for pediatric assessments. Adverse events will be coded by Medical Dictionary for Regulatory Activities and the type, incidence, severity, and relationship to investigational product will be summarized by treatment group. Other safety assessments will include: (1) occurrence of AESIs to include targeted AEs of hypersensitivity (including anaphylaxis), thrombocytopenia, and immune complex disease (e.g., vasculitis, endocarditis, neuritis, glomerulonephritis) following investigational product administration; and (2) occurrence of NOCDs following investigational product administration.

For PK analysis, following a single dose of nirsevimab, individual nirsevimab serum concentration data will be tabulated by treatment group along with descriptive statistics. PK parameters, e.g., Cₘₐₓ, AUC, apparent clearance, and terminal half-life, will be estimated using non-compartmental analysis, if data permit.

For ADA analysis, the incidence of ADA to nirsevimab will be assessed and summarized by number and percentage of subjects that are ADA positive by treatment group. The ADA titer will be listed by subject at different time points. The impact of ADA on PK, efficacy, and association with TEAEs and TESAEs will be assessed. These summaries will be conducted on the overall As-treated Population, AT1, and AT2, unless specified otherwise.

### Exploratory Endpoint Analyses

For analysis of healthcare resource utilization (HRU) and caregiver burden, the magnitude of HRU (e.g., number of admissions to hospitals and ICUs and duration of stay; number of subjects who require respiratory support and supplemental oxygen and the duration of use; number and types of outpatient visits, e.g., ER, urgent care, outpatient clinic; and number of prescription and over-the-counter medications and duration of use) will be summarized overall by treatment group, and for the following subgroups: subjects with at least one medically attended LRTI caused by RT-PCR-confirmed RSV, subjects with medically attended LRTI not caused by RSV, and subjects with non-protocol defined LRTIs, which may be further broken down by RSV status. These summaries will be conducted on ITT1 and ITT2 (if data permit).

Caregiver burden (e.g., caregiver missed work days, subject absence from day care) for subjects with medically attended LRTI caused by RT-PCR-confirmed RSV will be summarized by treatment group on ITT1 and ITT2 (if data permit).

### RSV Neutralizing Antibody and RSV Serology

For analysis of RSV neutralizing antibody levels afforded by nirsevimab, nirsevimab will be compared to maternal RSV neutralizing antibody levels and those elicited following infection in the placebo group. RSV sero-responses will be evaluated as a measure of RSV exposure in the placebo and nirsevimab groups.

### Monitoring RSV Resistance to Nirsevimab

Genotypic analysis of the full-length mature F protein will be conducted on all RSV-positive isolates confirmed centrally using the Lyra RSV+hMPV real-time RT-PCR assay manufactured by Quidel Corporation. RSV genotypic analysis will report amino acid changes in the mature F protein sequence compared to contemporary RSV A and RSV B reference strains. Phenotypic analyses will report changes in susceptibility of engineered recombinant RSV variants to nirsevimab and palivizumab neutralization compared to laboratory-derived reference viruses.

RSV LRTI Occurring from Day 152 to Day 361

The incidence of medically attended RSV LRTI (inpatient and outpatient) from Day 152 to Day 361 will be based on RSV test results (performed centrally via RT-PCR) and objective clinical LRTI criteria and will be summarized by treatment group on ITT1 and ITT2.

### Example 2: Nirsevimab Protects Healthy Late Preterm and Term Infants Against Respiratory Syncytial Virus

This Example describes the results from the Phase 3, randomized, double-blind, placebo-controlled study to evaluate the safety and efficacy of a single dose of nirsevimab against medically attended RSV-confirmed LRTI in healthy late preterm and term infants entering their first RSV season.

In this study, infants (gestational age ≥35 weeks) were randomized 2:1 to receive a single intramuscular injection of nirsevimab or placebo at the start of the RSV season. The primary efficacy endpoint was incidence of medically attended RSV LRTI through 150 days; the secondary endpoint was incidence of RSV-associated hospitalization through 150 days. The results of this study show that a single dose of nirsevimab administered before the RSV season protected healthy late preterm and term infants from medically attended RSV LRTI. Importantly, the results surprisingly show that the protective effect of a single dose of nirsevimab persisted for greater than five months, as many as twelve months. Accordingly, a single dose of nirsevimab, whether given within or outside the RSV season, is sufficient to provide protection against RSV infections for at least one five-month RSV season (e.g., for two RSV seasons). Therefore, the extended period of protection provided by a single dose of nirsevimab is able to protect subjects born outside of the RSV season, subjects who are experiencing an interrupted RSV season, and/or subjects experiencing RSV seasons longer than five months (e.g., six, seven, eight, nine or ten months).

### Methods

The clinical trial was carried out in accordance with the protocol set forth in Example 1 above. Further details are as follows.

### Participants

Healthy infants born late preterm or at term (gestational age ≥35 weeks 0 days at birth), ≤1 year of age, and entering their first RSV season were eligible for participation. Potential participants were excluded if they met national or other local criteria to receive commercial palivizumab, had any fever or acute illness within 7 days before randomization, or had RSV infection before, or at the time of, randomization.

### Trial Design

Participants were randomized 2:1 to receive one intramuscular injection of 50 mg or 100 mg (if <5 kg or ≥5 kg weight at dosing, respectively) of nirsevimab or saline placebo. Randomization was stratified by hemisphere (Northern or Southern) and by age (≤3 months, >3 months to ≤6 months, or >6 months). Medically attended respiratory illnesses were recorded throughout the study. The primary cohort included participants enrolled from 2019 across 150 sites (20 countries) in the Northern Hemisphere and from 2020 across 10 sites (1 country) in the Southern Hemisphere. See also FIG. 1.

### Endpoints

The primary efficacy endpoint was the incidence of medically attended RSV LRTI through 150 days after administration of nirsevimab or placebo; the secondary efficacy endpoint was the incidence of hospitalization due to this condition during the same period. All cases were confirmed by central laboratory testing of RSV using real-time, reverse-transcriptase-polymerase chain reaction (RT-PCR). The case definition of medically attended RSV LRTI for the primary endpoint is provided in Table 1, supra.

Subgroup analyses of the primary endpoint were prespecified according to hemisphere, age at randomization, sex, race, weight, and gestational age. Adverse events were graded by severity according to the National Cancer Institute Common Terminology Criteria for Adverse Events and coded by Medical Dictionary for Regulatory Activities. Hypersensitivity, including anaphylaxis, immune complex disease, and thrombocytopenia were designated as adverse events of special interest.

The pharmacokinetics (PK) of nirsevimab were determined as described in Domachowske et al., Pediatr Infect Dis J. (2018) 37:886-92. Serum samples were collected pre-dose, on Days 15 (replaced by Day 8 in some cases), 31, 151, and 361 post-dose and when participants were hospitalized for respiratory illnesses. Antidrug antibodies were assessed; a positive anti-nirsevimab antibody response was defined as a titer of ≥50, as described in Griffin et al., N Engl J Med. (2020) 383:415-25, and Domachowske, supra.

### Statistical Analyses

All analyses were based on the primary cohort. Efficacy analyses were performed in the intent-to-treat population (all randomized participants); safety analyses were based on the as-treated population (participants who received any investigational product). The sample size of 3,000 participants was selected for safety database consideration and provided >99% power for the primary efficacy endpoint. A sample size of approximately 1,500 participants for the primary efficacy analysis had ≥99% power to detect 70% relative risk reduction, at a two-sided significance level of 0.05, under the assumption of an 8% event rate in the placebo group.

Analysis of the primary endpoint was performed using a Poisson regression model with robust variance. To control for the overall type I error, a hierarchical approach was used; the secondary endpoint was tested only if statistical significance for the primary endpoint was shown.

For participants without an RSV LRTI who were not followed through 150 days post-dose, their event status was considered missing and was imputed with the observed event rate in the placebo group, with repeated imputation.

### Additional Analyses for Primary and Secondary Efficacy Endpoints

Follow-up after nirsevimab or placebo occurred by telephone (every 2 weeks through 150 days post-dose, monthly from 150-360 days post-dose and every 2 weeks 361-510 days post-dose) and in-person during trial site visits (days 8, 15, 31, 91, 151, and 361).

For medically attended lower respiratory tract events, RSV status was determined by central real-time, reverse-transcriptase-polymerase chain reaction (RT-PCR). After RSV detection by RT-PCR, RSV A and RSV B subtypes were determined by genotypic analysis of sequence changes in the mature F protein from all RSV-positive isolates/samples compared with contemporary RSV A and RSV B reference strains.

Pooled analyses of the RSV LRTI hospitalization endpoint were prespecified under a multiplicity-protected hierarchical testing strategy. The rationale for the pooled analysis, which consists of all intent-to-treat (ITT) participants from the Phase 2b trial D5290C00003 and ITT participants from the primary cohort of the present trial, was to assess the overall efficacy of RSV hospitalization in the target population (preterm and term infants). The pooled analysis consisting of the 860 ITT participants weighing <5 kg in the Phase 2b trial and ITT participants in the primary cohort of the present trial was conducted to assess the efficacy in all participants under the clinically efficacious exposure suggested by pharmacokinetic analysis. Combining these participants for analysis was justified based on the similar trial designs and disease similarity between the infant pediatric populations. Statistical testing of the null hypothesis that the incidence of RSV LRTI hospitalization between nirsevimab and placebo groups is the same would only be performed if the primary efficacy analysis had achieved a P-value that was ≤0.05.

More specifically, after the significance of the primary efficacy endpoint was demonstrated, the secondary efficacy endpoint would first be tested by pooling all ITT participants from the Phase 2b trial D5290C00003 and ITT participants from the primary cohort of the present study. If the significance were demonstrated (at 2-sided 0.05), the secondary efficacy endpoint would be further tested from pooling the 860 participants weighing <5 kg on Day 1 (i.e., 290 participants randomized to placebo and 570 participants randomized to nirsevimab) in the Phase 2b trial and ITT participants from the primary cohort of the present trial. If the significance were again demonstrated (at 2-sided 0.05), the secondary efficacy endpoint would be tested using ITT participants from the primary cohort of the present trial alone.

A Cochran-Mantel-Haenszel test based on the observed data was used as the secondary analysis model for the primary and secondary efficacy endpoints. Kaplan-Meier curves were generated for time to first medically attended RSV LRTI, where the hazard ratio and the corresponding 95% CI were obtained from stratified proportional-hazard model with the stratification factors (age at randomization) as the strata. For the subgroup analysis on the primary endpoint, within each level of a subgroup, the relative risk reduction and its 95% CI (mid-P adjusted) were estimated based on exact conditional method using PROC GENMOD with no strata.

For each of the impact endpoints (i.e., all medically attended LRTI due to RSV on any test result, all medically attended LRTI due to RSV on central test result, all medically attended LRTI of any cause, any respiratory illness due to RSV on any test result, any respiratory illness due to RSV on central test result, and any respiratory illness of any cause), efficacy (relative risk reduction of nirsevimab versus placebo), and the 95% CI were estimated based on Poisson regression with robust variance with the term of treatment. The estimate of the number of cases averted (and associated 95% CI) over the RSV season was calculated from the seasonal difference in the estimated number of cases between nirsevimab and placebo and expressed per 1000 infants immunized using bootstrap. Only the participants from the Northern Hemisphere were included in the analysis. A pooled analysis of efficacy against RSV hospitalization with the trial in preterm infants was prespecified with a multiplicity-protected hierarchical testing strategy.

### Results

Of 1490 randomized participants, 1478 (99.2%) received nirsevimab (n=987) or placebo (n=491). The incidence of medically attended RSV LRTI was 1.2% (n=12) in the nirsevimab group and 5.0% (n=25) in the placebo group, corresponding to efficacy of 74.5% (95% CI 49.6, 87.1; p<0.0001). The incidence of RSV-associated hospitalization was lower in the nirsevimab group compared with the placebo group (0.6%, (n=6) vs. 1.6% (n=8); efficacy 62.1% (95% CI-8.6, 86.8)). The incidence of very severe medically attended RSV LRTI was lower in the nirsevimab group compared with the placebo group (0.5%, (n=5) vs. 1.4%, (n=7); efficacy 64.2% (95% CI-12.1, 88.6)). Very severe RSV LRTI was an exploratory endpoint defined as those cases of hospitalization for medically attended RSV LRTI that required supplemental oxygen or intravenous fluids. For every 1,000 infants immunized, the estimated number of cases averted of all-cause LRTI was 93.6 (95% CI 63.0, 124.0) and estimated averted hospitalizations for all-cause respiratory illness were 17.7 (95% CI 2.0, 33.0). Adverse events were similar among trial groups. Further details of the clinical study results are described below.

### Population

Healthy infants born late preterm or at term (gestational age ≥35 weeks 0 days at birth), ≤1 year of age, and entering their first RSV season were eligible for participation. Potential participants were excluded if they met national or other local criteria to receive commercial palivizumab, had any fever or acute illness within 7 days before randomization, or had RSV infection before, or at the time of, randomization. Between Jul. 23, 2019, and Nov. 30, 2019, 1027 participants were enrolled in the NH and followed through the 2019/20 RSV season. Between Jan. 8, 2020, and Mar. 15, 2020, 462 participants were enrolled in South Africa and were followed through the expected 2020 season. One participant was enrolled in Japan before enrollment was paused due to the COVID-19 pandemic. In total, 1,490 participants were randomized in the primary cohort, and 1,478 (99.2%) received injections (nirsevimab: n=987; placebo: n=491). Overall, 1,465 and 1,367 participants completed the 150-day and the 360-day follow-up periods, respectively.

The trial population was predominantly infants born at term (86%). The median age was 2.60 months (range 0.03 to 11.10 months). Baseline characteristics were similar between treatment groups as shown in the table below.

TABLE 2
Baseline Characteristics of the Participants (ITT Population)*

| | | Nirsevimab | | Placebo | | Total* | |
|---|---|---|---|---|---|---|---|
| Variable | | (N = 994) | | (N = 496) | | (N = 1490) | |
| Age group, no. (%) | | | | | | | |
| ≤3 | months | 577 | (58.0) | 285 | (57.5) | 862 | (57.9) |
| >3 to ≤6 months | | 317 | (31.9) | 162 | (32.7) | 479 | (32.1) |
| >6 | months | 100 | (10.1) | 49 | (9.9) | 149 | (10.0) |
| Total | | 994 | | 496 | | 1490 | |
| Gestational age group, no. (%) | | | | | | | |
| ≥35 to <37 weeks | | 132 | (13.3) | 76 | (15.4) | 208 | (14.0) |
| ≥37 | weeks | 861 | (86.7) | 419 | (84.6) | 1280 | (86.0) |
| Total | | 993 | | 495 | | 1488 | |
| Female sex, no. (%) | | 464 | (46.7) | 257 | (51.8) | 721 | (48.4) |
| Weight group, no. (%) | | | | | | | |
| <5 | kg | 403 | (40.6) | 192 | (38.7) | 595 | (40.0) |
| ≥5 | kg | 589 | (59.4) | 304 | (61.3) | 893 | (60.0) |

| Total | 992 | | 496 | | 1488 | |
|---|---|---|---|---|---|---|
| Race or ancestry, no. (%)^{†} | | | | | | |
| American Indian or Alaska Native | 57 | (5.8) | 26 | (5.2) | 83 | (5.6) |
| Asian | 36 | (3.6) | 18 | (3.6) | 54 | (3.6) |
| Black or African American | 286 | (28.9) | 136 | (27.4) | 422 | (28.4) |
| Native Hawaiian/other Pacific Islander | 6 | (0.6) | 5 | (1.0) | 11 | (0.7) |
| White | 524 | (52.9) | 272 | (54.8) | 796 | (53.5) |
| Other/Multiple categories checked | 82 | (8.3) | 39 | (7.9) | 121 | (8.1) |
| Total | 991 | | 496 | | 1487 | |
| Hemisphere - no. (%) | | | | | | |
| Northern | 686 | (69.0) | 342 | (69.0) | 1028 | (69.0) |
| Southern | 308 | (31.0) | 154 | (31.0) | 462 | (31.0) |

*The “total” row includes the number of participants with non-missing data for the corresponding characteristic and was used as the denominator for calculating percentages for all categories.
^{†}Each race category counts participants who selected only that category. “Other/Multiple Categories Checked” counts participants who indicated a category other than those listed or checked more than one category.

### Efficacy

Medically attended RSV LRTI occurred in 1.2% (n=12/994) of participants randomized to receive nirsevimab and 5.0% (n=25/496) of participants who received placebo. This corresponded to an efficacy for nirsevimab of 74.5% (95% CI 49.6, 87.1; p<0.0001 by both Poisson regression and Cochran-Mantel-Haenszel test; Table 3), thus meeting the primary endpoint.

TABLE 3
Medically Attended LRTI and Hospitalization for RSV
LRTI Through 150 Days Post-Dose (ITT population)

| | Nirsevimab | | Placebo | | | | |
|---|---|---|---|---|---|---|---|
| | (N = 994) | | (N = 496) | | Efficacy* | | |
| End Points and Analyses | n (%) | | | | (95% CI) | | P-Value |
| Medically attended RSV LRTI | | | | | | | |
| Poisson regression with robust | 12 | (1.2) | 25 | (5.0) | 74.5 | (49.6, 87.1) | <0.0001 |
| variance | | | | | | | |
| Observed events | 15 | (1.5) | 6 | (1.2) | | | |
| Participants requiring imputation^{†} | 12 | (1.2) | 25 | (5.0) | | | <0.0001 |
| Cochran-Mantel-Haenszel test: | | | | | 76.1 | (52.7, 87.9) | |
| observed events | | | | | | | |
| Hospitalization for RSV LRTI | | | | | | | |
| Poisson regression with robust | | | | | 62.1 | (−8.6, 86.8) | 0.0708 |
| variance | | | | | | | |
| Observed events | 6 | (0.6) | 8 | (1.6) | | | |
| Participants with imputation of | 15 | (1.5) | 6 | (1.2) | | | |
| data^{†} | | | | | | | |
| Cochran-Mantel-Haenszel test: | 6 | (0.6) | 8 | (1.6) | 62.6 | (−7.3, 86.9) | 0.0571 |
| observed events | | | | | | | |

*Efficacy (relative risk reduction of nirsevimab versus placebo), the 95% CI, and P-value were estimated based on Poisson regression with robust variance (including stratification factor [age at randomization] as a covariate) obtained after missing data imputation.
^{†}Participants who had no events and were not followed through 150 days post-dose.

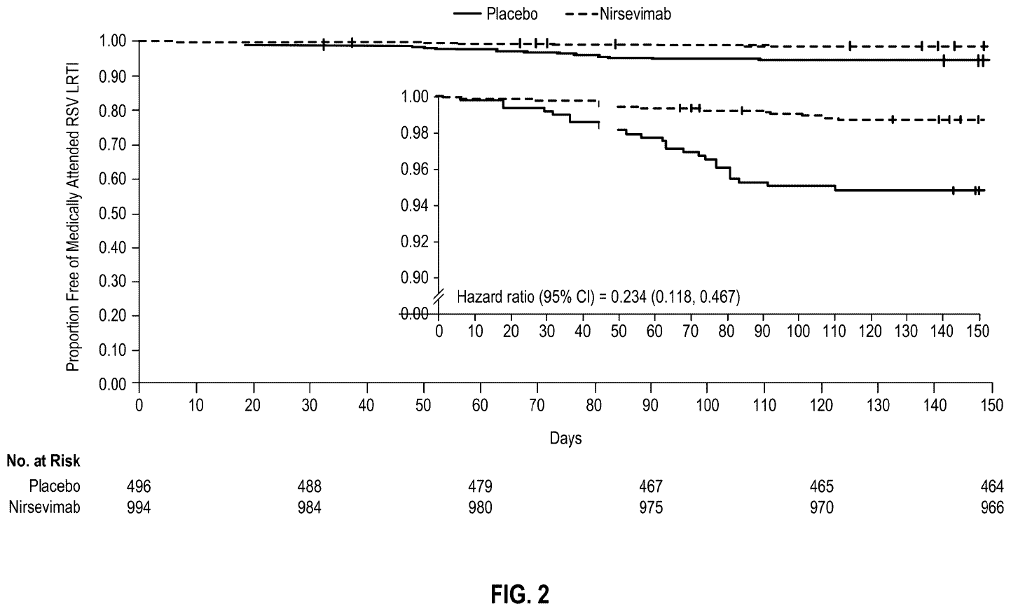

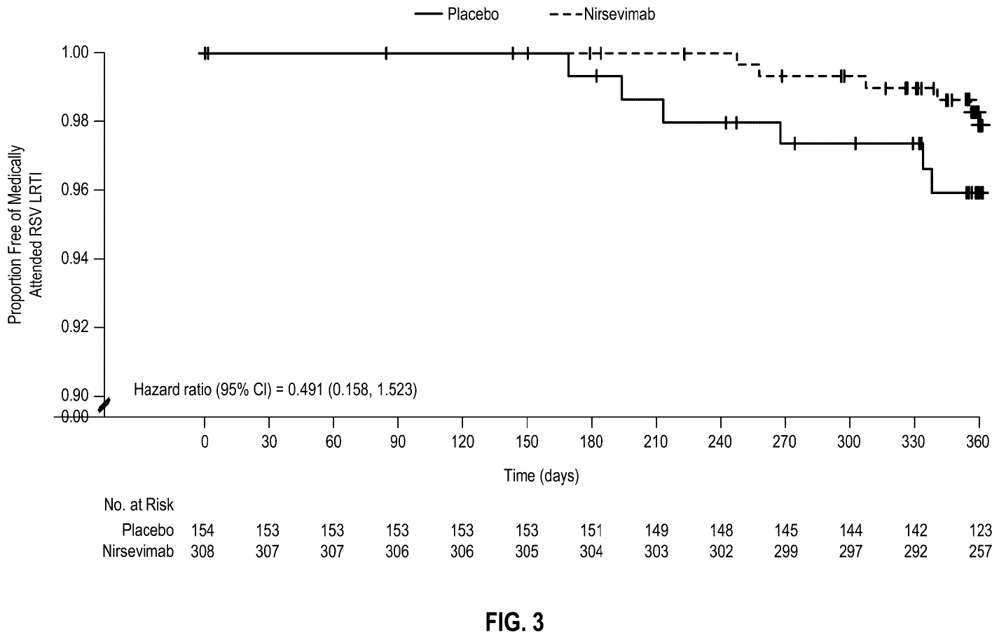

Time-to-event analysis confirmed that infants who received nirsevimab had a lower risk of medically attended RSV LRTI than infants who received placebo (hazard ratio, 0.23; 95% CI 0.12, 0.47) (FIG. 2). Of the RSV LRTI that occurred, all 12 in the nirsevimab group were RSV A, while in the placebo group, 21 were RSV A and 4 were RSV B. There was no contribution from South Africa to the primary efficacy estimate due to a reduction in RSV in South Africa during the COVID-19 pandemic (Tempia et al., Eurosurveillance (2021) (in press)). But unseasonal RSV transmission began after Day 151 with 12 cases occurring up to Day 361 (nirsevimab: 6/308 (1.9%) participants versus placebo 6/154 (3.9%) participants). A Kaplan-Meier plot of these data is provided in FIG. 3. One case occurred in the NH after Day 151, on Day 165, in the nirsevimab arm. The data in FIG. 3 show that the protective effect of a single dose of 50 mg nirsevimab persisted beyond the length of a typical RSV season—about five months or 150 days. The protective effect, as indicated by reduced medically attended RSV-associated LRTI, was evident for more than 240 days (about eight months), up to the end of the 360 days (about twelve months) study period (FIG. 3).

During the 150 days, hospitalization for RSV LRTI occurred in 6/994 (0.6%) participants in the nirsevimab group and 8/496 (1.6%) in the placebo group, corresponding to an estimate of efficacy of 62.1% (95% CI-8.6, 86.8; P=0.0708, Poisson regression) (Table 3). Health care usage associated with these cases is provided in Table 4.

TABLE 4
Healthcare Usage Associated with RSV LRTI Hospitalization

| | Placebo | Nirsevimab |
|---|---|---|
| Healthcare Usage | (n = 8) | (n = 6) |
| Days hospitalized, mean (SD) | 4 (2.2) | 7.2 (4.6) |
| Required oxygen, n | 6 | 4 |
| Required CPAP or HFNC, n | 1 | 1 |
| Required IV fluids, n | 1 | 1 |

CPAP: continuous positive airway pressure; HFNC: high flow nasal cannula; SD: standard deviation.

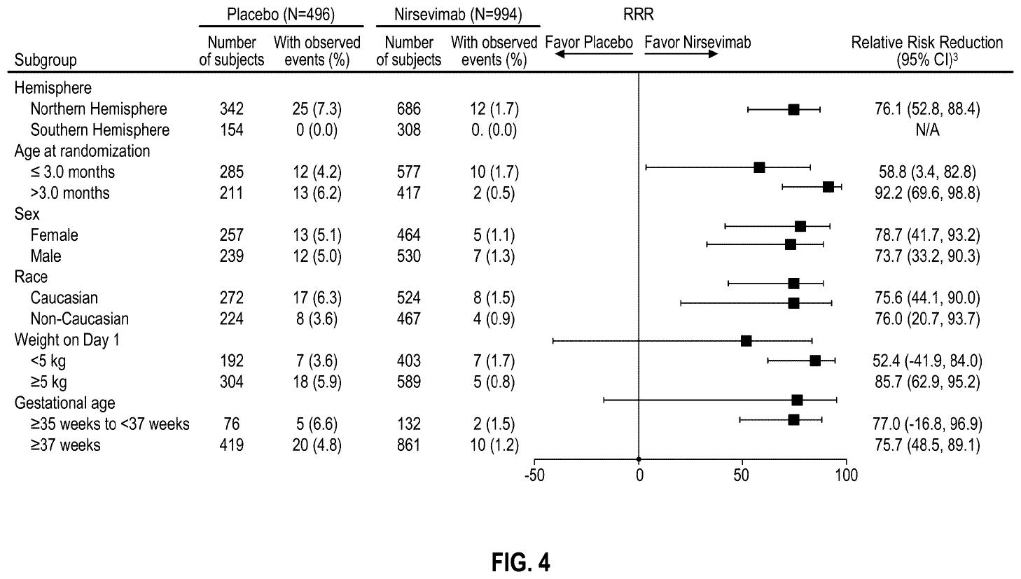

Subgroup analyses according to hemisphere, age at randomization, sex, race, weight, and gestational age showed consistent efficacy favoring nirsevimab. However, trends of lower efficacy were observed in those of younger age (≤3.0 months vs. >3.0 months) and lower weight (<5 kg vs. ≥5 kg; FIG. 4).

Impact was assessed in the NH, where a typical RSV season occurred prior to the COVID-19 pandemic becoming established. A demonstrable impact was seen on all cause LRTI and hospitalization for any respiratory illness: for 1,000 infants immunized, the number of cases averted for LRTI of any cause was estimated to be 93.6 (95% CI. 63.0, 124.0) and the number of hospitalizations averted for respiratory illness of any cause was estimated to be 17.7 (95% CI 2.0, 33.0). The impact of nirsevimab in the ITT population from the Northern Hemisphere (NH) is summarized in the table below.

TABLE 5
Impact of Nirsevimab in ITT population from the Northern Hemisphere

| | | Efficacy* | | Cases Averted | |
|---|---|---|---|---|---|
| | | Relative Risk | | per 1000 | |
| | % Affected | Reduction (95% | | Immunized^{†}, | |
| Medical Events | (Placebo) | CI) | | n (95% CI) | |
| All medically attended LRTI due | 10.8 | 77.0 | (59.8, 86.8) | 83.4 | (62.0, 105.0) |
| to RSV on any test result^{‡§} | | | | | |
| All medically attended LRTI due | 9.6 | 77.2 | (58.7, 87.5) | 74.7 | (53.0, 95.0) |
| to RSV on central test result^{‡} | | | | | |
| All medically attended LRTI of | 18.1 | 51.5 | (32.6, 65.2) | 93.6 | (63.0, 124.0) |
| any cause^{‡} | | | | | |
| Hospitalization for any | 3.2 | 59.0 | (2.1, 82.9) | 19.0 | (5.5, 32.0) |
| respiratory illness due to RSV on | | | | | |
| any test result^{§} | | | | | |
| Hospitalization for any | 2.6 | 61.1 | (−3.7, 85.4) | 16.1 | (4.5, 28.0) |
| respiratory illness due to RSV on | | | | | |
| central test result^{¶} | | | | | |
| Hospitalization for any | 4.1 | 42.8 | (−15.8, 71.7) | 17.7 | (2.0, 33.0) |
| respiratory illness of any cause^{¶} | | | | | |

*Efficacy (Relative risk reduction of nirsevimab versus placebo) and the 95% CI were estimated based on Poisson regression with robust variance with the term of treatment.
^{†}Number of cases averted was calculated from the difference in the estimated number of cases between nirsevimab and placebo and expressed per 1,000 infants immunized. The 95% CI was estimated by bootstrapping, using the 2.5 and 97.5 percentiles of 1,000 replicates
obtained by sampling participants.
^{‡}All is used to indicate a medically attended lower respiratory tract infection that does and does not meet the definition applied in the primary endpoint.
^{§}Any test result refers to either the central reference test for the trial or a local test performed in the context of clinical care.
^{¶}Any respiratory illness includes both upper and lower respiratory tract infections.

### Pharmacokinetics

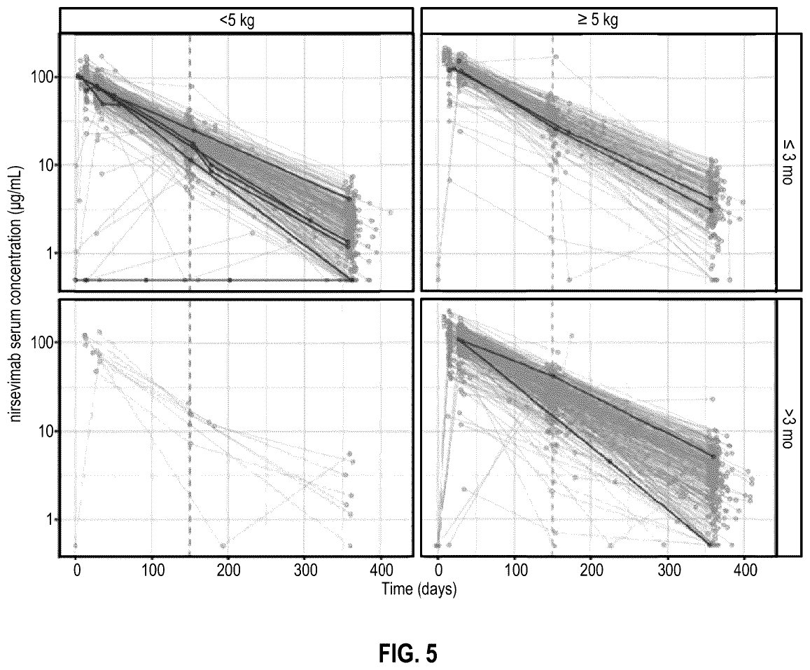

Serum concentrations of nirsevimab decreased in proportion to the concentration beyond Day 31 without signs of nonlinearity (FIG. 5). The mean (SD) half-life of nirsevimab was 66.9 (10.9) days and was similar for both weight subgroups. On Day 151, mean (SD) nirsevimab serum concentrations were 19.6 (7.7) and 31.1 (13.7) μg/mL in the <5 kg and ≥5 kg subgroups, respectively. Four participants in the nirsevimab group had no quantifiable serum concentrations at any timepoint; dosing errors were not reported but may have occurred.

### Antidrug Antibodies

Postbaseline antidrug antibodies were detected in 58 of 951 (6.1%) and 5 of 473

(1.1%) of the participants with available results to Day 361 who received nirsevimab or placebo, respectively. During the 150-day period after administration of the dose, 2 of 12 nirsevimab recipients who had a medically attended RSV LRTI had antidrug antibodies detected by laboratory testing with a titer of 400 on Days 151 and 361. Nirsevimab recipients who were positive for antidrug antibodies post baseline had a similar safety profile to nirsevimab recipients without antidrug antibodies.

### Safety and Adverse Event Profile

The types and frequencies of adverse events occurring during the trial were similar in both groups. Treatment-emergent adverse events (TEAEs) 360 days post-dose that occurred in the as-treated population are summarized in Table 6.

Most adverse events occurring during treatment were grade 1 or 2 in severity. Adverse events of grade 3 severity or higher were reported in 3.6% (36/987) of those who received nirsevimab and 4.3% (21/491) of those who received placebo. The incidence of adverse events within 1 day of dose administration in the nirsevimab group was low (1.8% of participants in the nirsevimab group and 0.6% of participants in the placebo group). These adverse events were all grade 1 severity and were managed by parents at home with treatments available over the counter. The incidence of adverse events within 7 days of dose administration was similar in both groups (13.4% participants in the nirsevimab group and 12.8% of participants in the placebo group; Table 6). In this period, the incidence of adverse events in the System Organ Class of general disorders and administration-site conditions was low, occurring in 0.4% (2/491) of participants in the placebo group and 0.6% (6/987) of participants in the nirsevimab group: these were pyrexia (3 participants), discomfort (2 participants) and local injection site pain or swelling (3 participants).

TABLE 6
TEAEs Through 360 Days Post-dose in the As-Treated Population

| | Nirsevimab | | Placebo | | Total | |
|---|---|---|---|---|---|---|
| | (N = 987) | | (N = 491) | | (N = 1478) | |
| Variable | Participants,* n (%) | | | | | |
| At least one event | 863 | (87.4) | 426 | (86.8) | 1289 | (87.2) |
| Occurring ≤ 1 day after the dose | 18 | (1.8) | 3 | (0.6) | 21 | (1.4) |
| Occurring ≤ 3 days postdose | 56 | (5.7) | 23 | (4.7) | 79 | (5.3) |
| Occurring ≤ 7 days after the dose | 132 | (13.4) | 63 | (12.8) | 195 | (13.2) |
| At least one event considered related to trial | 10 | (1.0) | 7 | (1.4) | 17 | (1.2) |
| drug | | | | | | |
| At least one event of ≥ grade 3 severity^{†} | 36 | (3.6) | 21 | (4.3) | 57 | (3.9) |
| Any adverse event with outcome death (grade | 3 | (0.3) | 0 | | 3 | (0.2) |
| 5 severity) | | | | | | |
| At least one serious^{‡} event | 67 | (6.8) | 36 | (7.3) | 103 | (7.0) |
| Considered related to trial drug | 0 | | 0 | | 0 | |
| At least one adverse event of special interest^{§} | 1 | (0.1) | 0 | | 1 | (0.1) |
| At least one event related to COVID-19 | 7 | (0.7) | 7 | (1.4) | 14 | (0.9) |
| At least one COVID-19 confirmed^{¶} event | 6 | (0.6) | 6 | (1.2) | 12 | (0.8) |
| At least one COVID-19 suspected event | 1 | (0.1) | 1 | (0.2) | 2 | (0.1) |

*Participants with multiple events in the same category are counted once in that category. Participants with events in more than one category are counted once in each of those categories.
^{†}Grade 3: Severe; Grade 4: Life-threatening; Grade 5: Fatal.
^{‡}Serious adverse event criteria: death, life-threatening, required inpatient hospitalization, prolongation of existing hospitalization, persistent or significant disability/incapacity, and important medical event.
^{§}Based on investigator assessment. Adverse events of special interest were hypersensitivity, immune complex disease, and thrombocytopenia.
^{¶}COVID-19 confirmed events include COVID-19 positive asymptomatic and symptomatic events. Events that occurred after 360 days post-dose were excluded. One participant was ongoing and had not reached Day 361 at the time of the data cut-off.

Serious adverse events were reported in 6.8% (67/987) of the participants who received nirsevimab and 7.3% (36/491) of those who received placebo. The investigators considered none to be related to the investigational product. A single adverse event of special interest was reported in one participant in the nirsevimab group who had a generalized macular rash without any systemic features 6 days after dosing, and this event to be treatment-related. No anaphylaxis or other serious hypersensitivity reactions were reported.

Three deaths occurred through Day 361 (all in the nirsevimab group). One death of unknown cause occurred on Day 140 in a participant with failure to thrive. An underlying chronic illness undiagnosed before death was suspected based on reported adverse events of recurrent vomiting, hypoglycemia, and anemia. Two deaths (on Days 143 and 338) were attributed to gastroenteritis in participants who did not have a healthcare visit for the illness. No deaths were known to be due to RSV or considered by the investigator to be related to nirsevimab. The above study demonstrates that a single dose of the monoclonal antibody, nirsevimab, provides substantial protection against medically attended RSV LRTI when given to healthy late preterm and term infants before the RSV season. Efficacy against all medically attended LRTI of any cause has been observed. Furthermore, nirsevimab was well-tolerated: only 1% of participants reported an adverse event related to treatment and local reactogenicity and fever occurred rarely.

The nirsevimab 90% effective concentration determined preclinically was 6.8 μg/mL (Zhu et al., supra). The PK data herein support that protective antibody levels extend to 150 days post-dose across age/body weight subgroups. The data from South Africa are supportive of continuing protection beyond five months, the typical length of an RSV season.

In conclusion, this trial demonstrated that nirsevimab, a monoclonal antibody against RSV with an extended half-life, is efficacious for preventing medically attended RSV LRTI in healthy late preterm and term infants. Use of nirsevimab could mitigate the substantial burden of disease and potentially prevent long-term consequences associated with RSV illness in the general infant population.

### Further Analysis of Pooled Data

Table 7, below, shows demographics and baseline characteristics of subjects in the Phase IIb and Phase III (MELODY) studies, with 786 subjects in the placebo group and 1564 subjects in the nirsevimab-treated group. The studies evaluated an ethnically diverse population, the median subject age was 2 months (ranging from 1 day to 11 months of age), and the demographics and baseline characteristics were balanced between treatment arms.

TABLE 7
Demographics and Baseline Characteristics for Further Analysis of Pooled Data

| | | |
|---|---|---|
| | Placebo | Nirsevimab |
| Characteristic | (n = 786) | (n = 1564) |

Infants per trial (proposed dose), n (%)

| | | | | |
|---|---|---|---|---|
| Phase 2b <5 kg | 290 | (36.9) | 570 | (36.4) |
| MELODY | 496 | (63.1) | 994 | (63.6) |
| Female, n (%) | 397 | (50.5) | 736 | (47.1) |
| Age, median months (range) | 2.00 | (0.03-10.97) | 2.02 | (0.03-11.10) |
| ≤3.0 months, n (%) | 531 | (67.6) | 1066 | (68.2) |
| >3.0 to ≤6.0 months, n (%) | 204 | (26.0) | 398 | (25.4) |
| >6.0 months, n (%) | 51 | (6.5) | 100 | (6.4) |

Weight group on Day 1, n (%)

| | | | | | |
|---|---|---|---|---|---|
| <5 | kg | 482 | (61.3) | 973 | (62.3) |
| ≥5 | kg | 304 | (38.7) | 589 | (37.7) |

Gestational age group, n (%)

| ≥29 to ≤32 weeks | | 116 | (14.8) | 219 | (14.0) |
|---|---|---|---|---|---|
| >32 to <35 weeks | | 175 | (22.3) | 344 | (22.0) |
| ≥35 to <37 weeks | | 76 | (9.7) | 139 | (8.9) |
| ≥37 | weeks | 419 | (53.3) | 861 | (55.1) |

Race or ethnic group, n (%)

| | | | | |
|---|---|---|---|---|
| American Indian or Alaska Native | 26 | (3.3) | 57 | (3.7) |
| Asian | 24 | (3.1) | 39 | (2.5) |
| Black or African American | 176 | (22.4) | 406 | (26.0) |
| Native Hawaiian or other Pacific Islander | 8 | (1.0) | 12 | (0.8) |
| White | 478 | (60.8) | 919 | (58.9) |
| Other | 70 | (8.9) | 109 | (7.0) |
| Multiple categories | 4 | (0.5) | 18 | (1.2) |

TABLE 8
Indicators of disease severity in cases of medically attended RSV LRTI through 150 days post-dose

| | | | | MA RSV LRTI with | | | | | |
|---|---|---|---|---|---|---|---|---|---|
| | MA RSV LRTI | | | hospitalization | | | MA RSV LRTI (very severe) | | |
| | Placebo | Nirsevimab | Total | Placebo | Nirsevimab | Total | Placebo | Nirsevimab | Total |
| n (%) | (n = 51) | (n = 19) | (N = 70) | (n = 21) | (n = 9) | (N = 30) | (n = 18) | (n = 5) | (N = 23) |
| Increased | 30 | 11 | 41 | 14 | 5 | 19 | 13 | 2 | 15 |
| respiratory | (58.8) | (57.9) | (58.6) | (66.7) | (55.6) | (63.3) | (72.2) | (40.0) | (65.2) |
| rate | | | | | | | | | |
| Hypoxemia | 20 | 5 | 25 | 11 | 3 | 14 | 10 | 3 | 13 |
| | (39.2) | (26.3) | (35.7) | (52.4) | (33.3) | (46.7) | (55.6) | (60.0) | (56.5) |
| O₂ <92% | 10 | 4 | 14 | 7 | 3 | 10 | 7 | 3 | 110 |
| | (19.6) | (21.1) | (20.0) | (33.3) | (33.3) | (33.3) | (38.9) | (60.0) | (43.5) |
| O₂ <90% | 5 | 2 | 7 | 5 | 2 | 7 | 5 | 2 | 7 |
| | (9.8) | (10.5) | (10.0) | (23.8) | (22.2) | (23.3) | (27.8) | (40.0) | (30.4) |
| Acute HVF | 3 | 1 | 4 | 3 | 1 | 4 | 3 | 1 | 4 |
| | (5.9) | (5.3) | (5.7) | (14.3) | (11.1) | (13.3) | (16.7) | (20.0) | (17.4) |
| NOA | 3 | 0 | 3 | 3 | 0 | 3 | 3 | 0 | 3 |
| | (5.9) | (0.0) | (4.3) | (14.3) | (0.0) | (10.0) | (16.7) | (0.0) | (13.0) |
| Nasal flaring | 12 | 6 | 18 | 9 | 4 | 13 | 8 | 2 | 10 |
| | (23.5) | (31.6) | (25.7) | (42.9) | (44.4) | (43.3) | (44.4) | (40.0) | (43.5) |
| Retractions | 42 | 12 | 54 | 21 | 7 | 28 | 18 | 4 | 22 |
| | (82.4) | (63.2) | (77.1) | (100.0) | (77.8) | (93.3) | (100.0) | (80.0) | (95.7) |
| Grunting | 3 | 5 | 8 | 2 | 3 | 5 | 2 | 3 | 5 |
| | (5.9) | (26.3) | (11.4) | (9.5) | (33.3) | (16.7) | (11.1) | (60.0) | (21.7) |
| Dehydration | 3 | 1 | 4 | 3 | 1 | 4 | 3 | 1 | 4 |
| | (5.9) | (5.3) | (5.7) | (14.3) | (11.1) | (13.3) | (16.7) | (20.0) | (17.4) |

| | | | | MA respiratory illness (all | | |
|---|---|---|---|---|---|---|
| | MA LRTI (all cause) | | | cause) with hospitalization | | |
| | Placebo | Nirsevimab | Total | Placebo | Nirsevimab | Total |
| n (%) | (n = 149) | (n = 191) | (n = 340) | (n = 51) | (n = 57) | (N = 108) |
| Increased | 63 | 92 | 155 | 25 | 34 | 59 |
| respiratory | (42.3) | (48.2) | (45.6) | (49.0) | (59.6) | (54.6) |
| rate | | | | | | |
| Hypoxemia | 33 | 26 | 59 | 18 | 19 | 37 |
| | (22.1) | (13.6) | (17.4) | (35.3) | (33.3) | (34.3) |
| O₂ <92% | 17 | 15 | 32 | 11 | 15 | 26 |
| | (11.4) | (7.9) | (9.4) | (21.6) | (26.3) | (24.1) |
| O₂ <90% | 10 | 12 | 22 | 8 | 13 | 21 |
| | (6.7) | (6.3) | (6.5) | (15.7) | (22.8) | (19.4) |
| Acute HVF | 5 | 7 | 12 | 6 | 8 | 14 |
| | (3.4) | (3.7) | (3.5) | (11.8) | (14.0) | (13.0) |
| NOA | 7 | 2 | 9 | 7 | 2 | 9 |
| | (4.7) | (1.0) | (2.6) | (13.7) | (3.5) | (8.3) |
| Nasal flaring | 18 | 25 | 43 | 13 | 14 | 27 |
| | (12.1) | (13.1) | (12.6) | (25.5) | (24.6) | (25.0) |
| Retractions | 64 | 70 | 134 | 35 | 20 | 75 |
| | (43.0) | (36.6) | (39.4) | (68.6) | (70.2) | (69.4) |
| Grunting | 7 | 5 | 12 | 4 | 2 | 6 |
| | (4.7) | (2.6) | (3.5) | (7.8) | (3.5) | (5.6) |
| Dehydration | 6 | 6 | 12 | 7 | 6 | 13 |
| | (4.0) | (3.1) | (3.5) | (13.7) | (10.5) | (12.0) |

For infants with multiple events, only the first event is included in the analysis.
HVF = acute hypoxic or ventilatory failure; LRTI = lower respiratory tract infection; MA = medically attended; NOA = new onset apnea; RSV = respiratory syncytial virus.

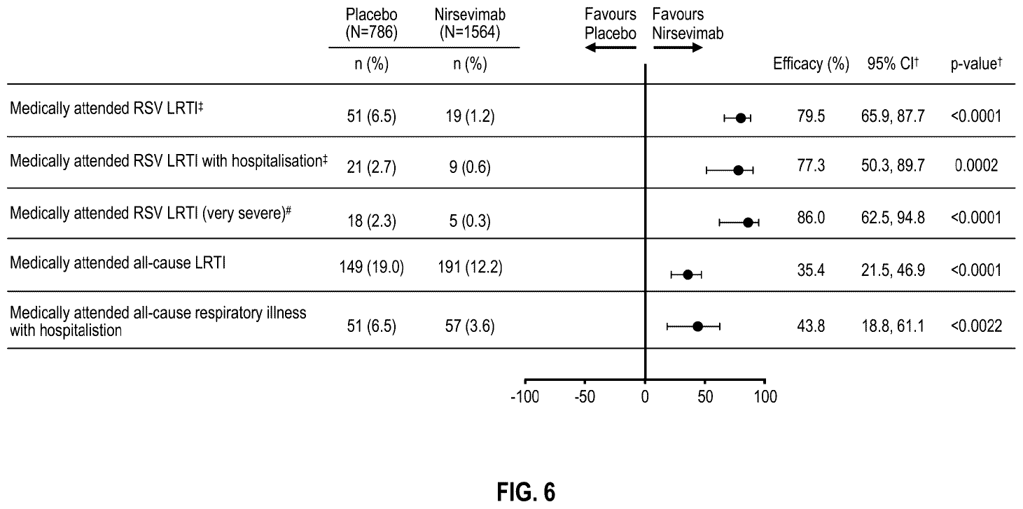

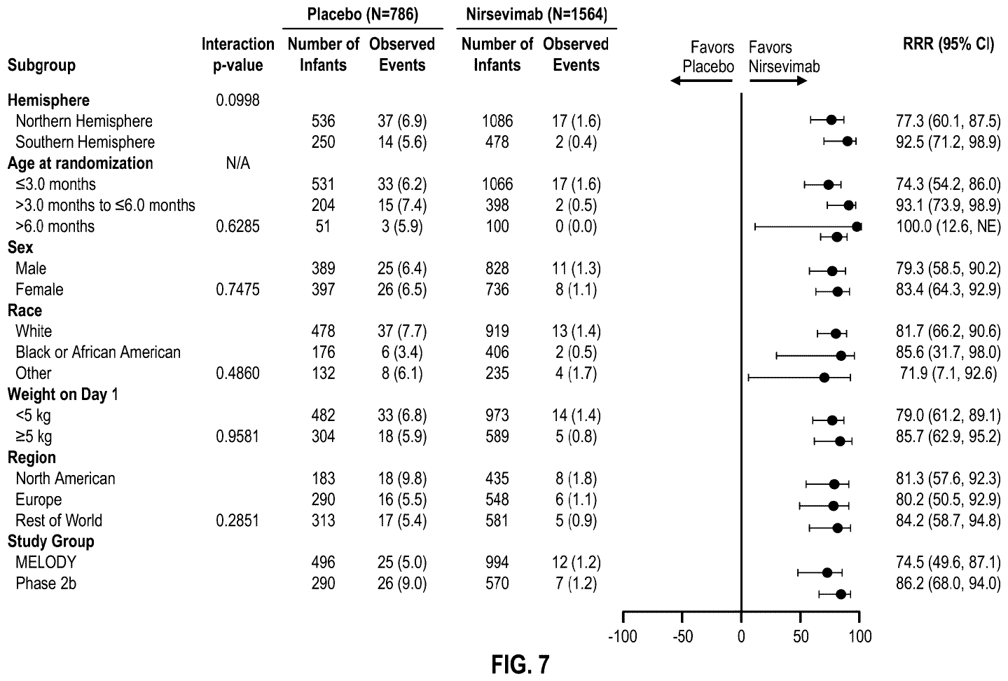

Nirsevimab showed consistent efficacy across medically attended RSV lower respiratory tract infection of different severities (FIG. 6 and Table 8) and across subgroups (FIG. 7). Further, nirsevimab showed efficacy against all-cause medically attended lower respiratory tract infection and against all-cause respiratory illness with hospitalization. See FIG. 6. While the demonstrable efficacy against all-cause LRTI does not preclude ‘replacement’ of RSV by another pathogen, it does provide reassurance that any potential for ‘replacement’ is outweighed by the overall treatment benefit of nirsevimab.

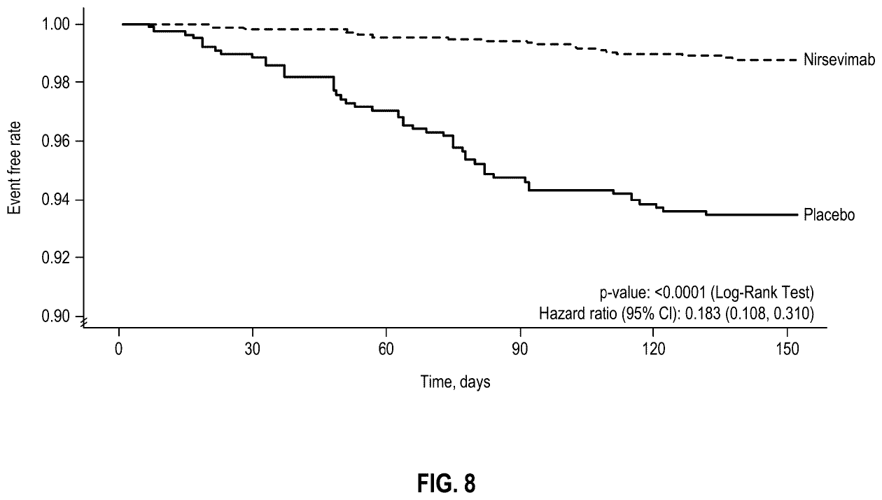

As shown in FIG. 8, nirsevimab's efficacy against medically attended lower respiratory tract infection was consistent over 150 days (5 months).

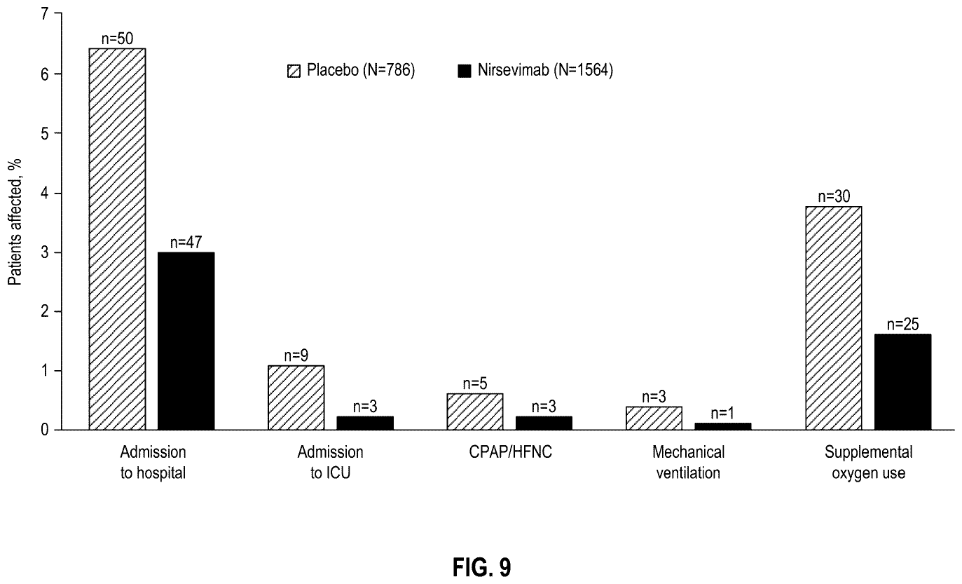

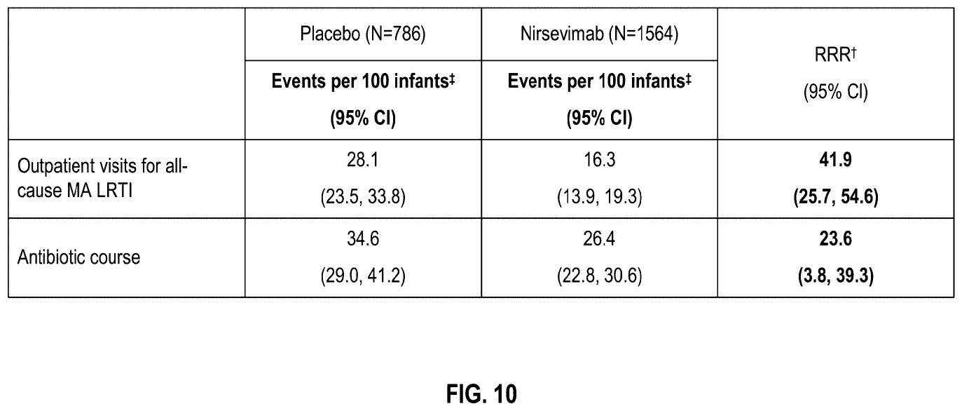

Subjects' inpatient health care resource utilization trended lower with nirsevimab than with placebo. Measures of inpatient health care utilization included hospital admission, intensive care unit (ICU) admission, continuous positive airway pressure (CPAP)/high-flow nasal cannula (HFNC), mechanical ventilation, and supplemental oxygen use. See FIG. 9. Nirsevimab treatment was also associated with fewer outpatient visits and less antibiotic use compared to placebo. See FIG. 10.

These results are consistent with results described above that nirsevimab is a long-acting antibody that offers protection against RSV over at least a season with a single dose, with efficacy across disease severities of RSV lower respiratory tract infection, against all-cause medically attended lower respiratory tract infection, and against hospitalization due to all-cause lower respiratory tract infection. Nirsevimab also has associated benefits of reducing inpatient care, outpatient visits, and antibiotic usage.

### Example 3: Nirsevimab for the Prevention of RSV: Neutralising Antibody Levels Following a Single Dose

Two global, placebo-controlled studies showed that nirsevimab reduced medically attended RSV LRTI versus placebo throughout the RSV season (Phase III: MELODY, healthy term and late preterm infants, 74.5%; Phase IIb: healthy preterm infants, 70.1% (95% CI, 52.3 to 81.2 p<0.001)). RSV neutralising antibodies (RSV Nab) were measured from these studies through Day 361.

As described above, infants were randomised 2:1 to receive one intramuscular injection of nirsevimab or placebo, before their first RSV season. Serum samples collected pre- and post-dose were tested in a validated RSV neutralisation assay; RSV Nab levels are reported in international units (IU)/mL.

RSV Nab levels were quantitated by interpolating from a serially diluted pooled serum Reference Standard Curve calibrated to the 1st International Standard for antiserum to RSV-NIBSC 16/284 (also referred to as the WHO RSV A Reference standard).

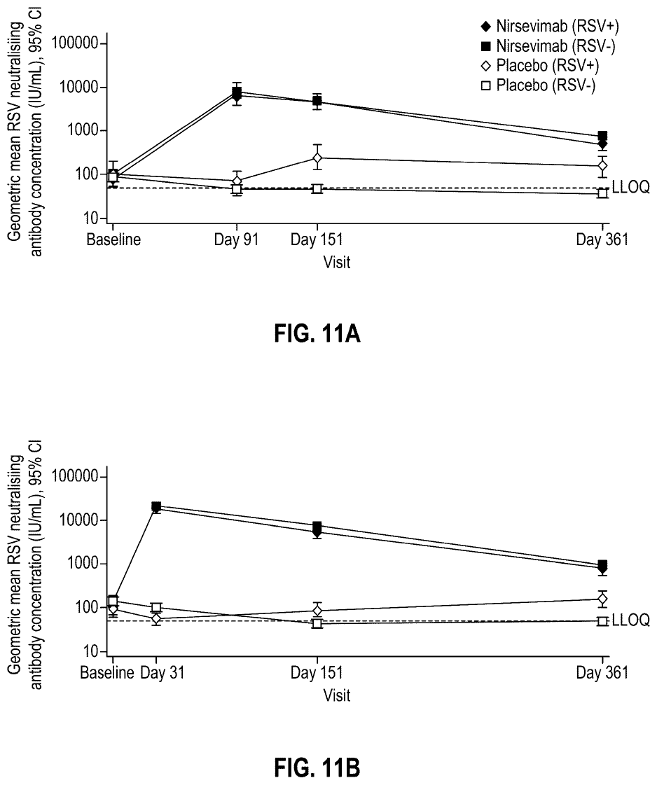

Overall, 1402 infants from MELODY and 741 infants from Phase IIb had available data. Baseline geometric mean RSV Nab levels were similar in both studies (MELODY, 134 IU/mL; Phase IIb, 87 IU/mL). At Day 151, nirsevimab recipients exhibited RSV Nab levels approximately 50-fold higher (MELODY, 6901 IU/mL; Phase IIb, 4799 IU/mL) versus baseline, with highest levels sampled at Day 31 in MELODY (19711 IU/mL) and at Day 91 in Phase IIb (8479 IU/mL); levels remained >5-fold higher through Day 361 (MELODY, 978 IU/mL; Phase IIb, 739 IU/mL). At Day 361, placebo recipients with no confirmed RSV infection during the studies had RSV Nab levels of 38-48 IU/mL; nirsevimab recipients had RSV Nab levels of 757-982 IU/mL, >19-fold higher than placebo recipients with no confirmed RSV infection. See FIGS. 11A-11B.

In conclusion, following immunisation with nirsevimab, RSV Nab levels at Day 151 were approximately 50-fold higher than baseline levels. RSV Nab levels remained high through Day 361, suggesting protection beyond Day 151.

### Further Analysis of Nab Data

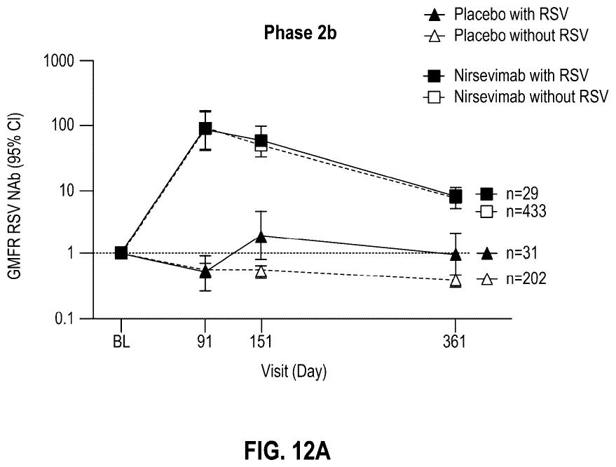

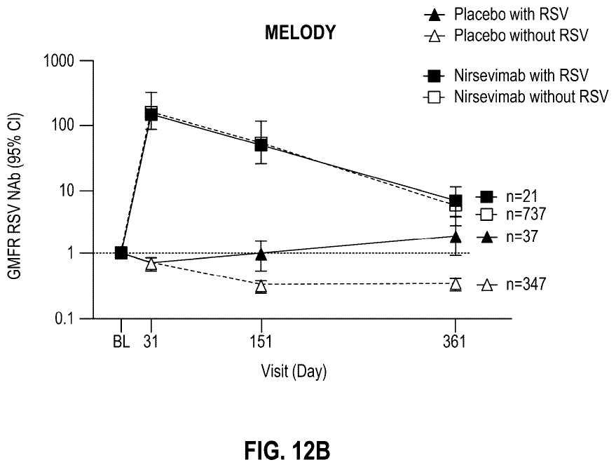

An updated analysis of data from the 1402 infants from MELODY and the 741 infants from Phase IIb was performed. At Day 151, nirsevimab recipients exhibited RSV Nab levels approximately 50-fold higher (MELODY, 6901 IU/mL; Phase IIb, 4799 IU/mL) versus baseline, with the highest levels sampled at Day 31 in MELODY (19737 IU/mL), and at Day 91 in Phase IIb (8479 IU/mL). RSV Nab levels remained >7-fold higher than baseline through Day 361 (MELODY, 978 IU/mL; Phase IIb, 739 IU/mL). At Day 361, placebo recipients with no confirmed RSV infection during the studies had low RSV Nab levels of 38-48 IU/mL compared with 151-162 IU/mL in recipients with confirmed RSV infection; nirsevimab recipients had RSV Nab levels of 757-979 IU/mL, >19-fold higher than placebo recipients with no confirmed RSV infection and >3 fold higher than placebo recipients with a confirmed RSV infection. See FIGS. 12A-12B.

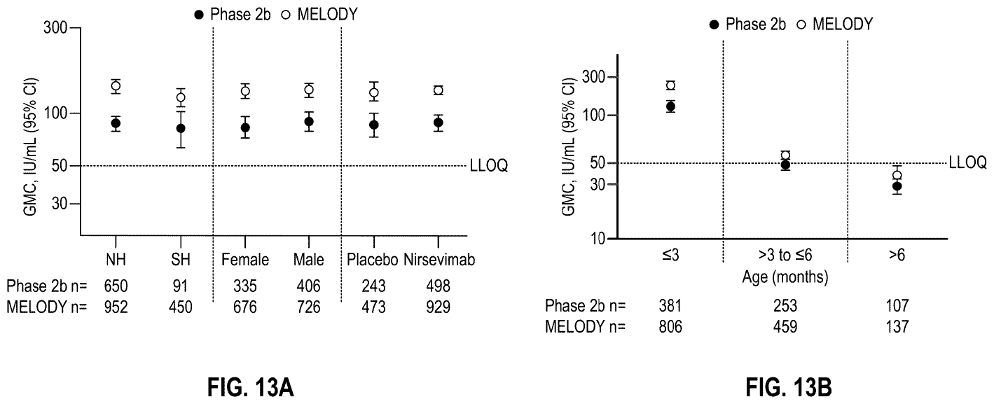

Within each study, similar levels of RSV Nab were observed at baseline, regardless of hemisphere, gender, or treatment group. Comparing the two studies, lower RSV Nab levels were observed in preterm infants from the Phase IIb study versus late-preterm and term infants in MELODY. Infants >6 months of age at baseline had the lowest RSV Nab levels. See FIGS. 13A-13B.

### Example 4: Pooled Efficacy of Nirsevimab Against RSV LRTI in Preterm and Term Infants

Nirsevimab reduced medically attended (MA) respiratory syncytial virus (RSV) lower respiratory tract infection (LRTI) incidence in two double-blind, placebo-controlled studies (Phase IIb [NCT02878330]: very and moderately preterm infants ≥29 to <35 weeks gestational age [wkGA], efficacy 70.1%; Phase III: MELODY [NCT03979313], healthy term and late preterm infants ≥35 wkGA, efficacy 74.5%). This Example reports a pooled efficacy analysis of nirsevimab in term and preterm infants >29 wkGA through Day 151.

Infants were randomised 2:1 to receive either an intramuscular injection of nirsevimab (<5 kg, 50 mg; ≥5 kg, 100 mg) or placebo, before their first RSV season. Data were pooled from the Phase IIb and MELODY studies for those infants under the optimized dosing regimen (i.e., infants <5 kg at dosing and receiving the 50 mg dose from Phase IIb and all infants in MELODY) to evaluate efficacy (relative risk reduction versus placebo) against varying severities of MA RSV LRTI, including hospitalization due to RSV LRTI.

Only infants <5 kg were included from Phase IIb, as infants received 50 mg regardless of age and weight in this study. MA RSV LRTI was defined as RSV PCR positive on central laboratory testing, a sign of LRT involvement of at least one of rhonchi, rales, crackles or wheeze, and at least one sign of severity including increased respiratory rate (≥60 breaths/min, age <2 months; >50 breaths/min, age 2-6 months; >40 breaths/min, age >6 months), hypoxemia at room air (O₂ saturation <95% at ≤1800 m, <92% at >1800 m) or clinical signs of respiratory distress (new onset apnea, retractions, grunting, nasal flaring, acute hypoxic or ventilatory failure, dehydration due to respiratory distress).

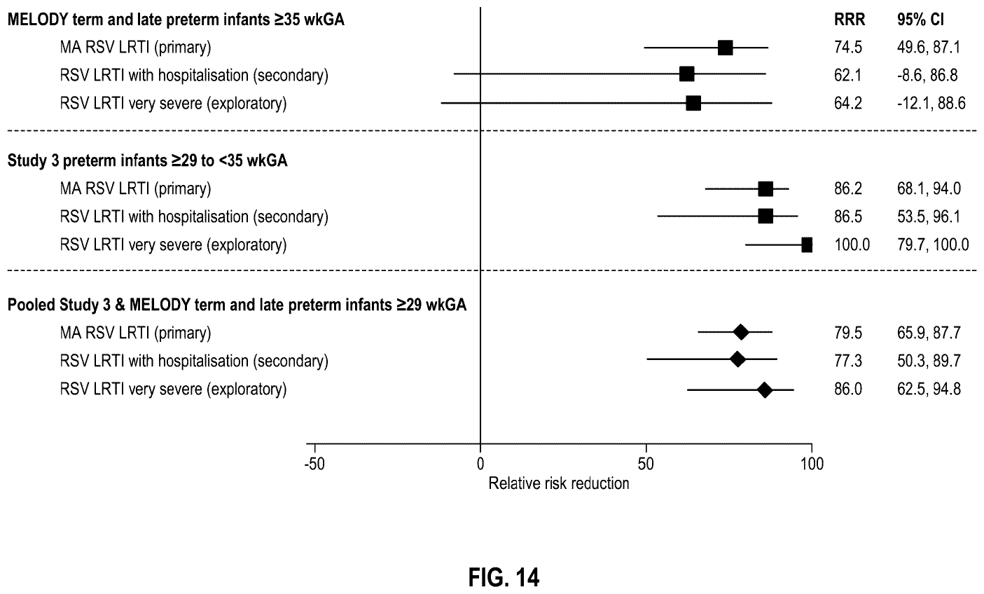

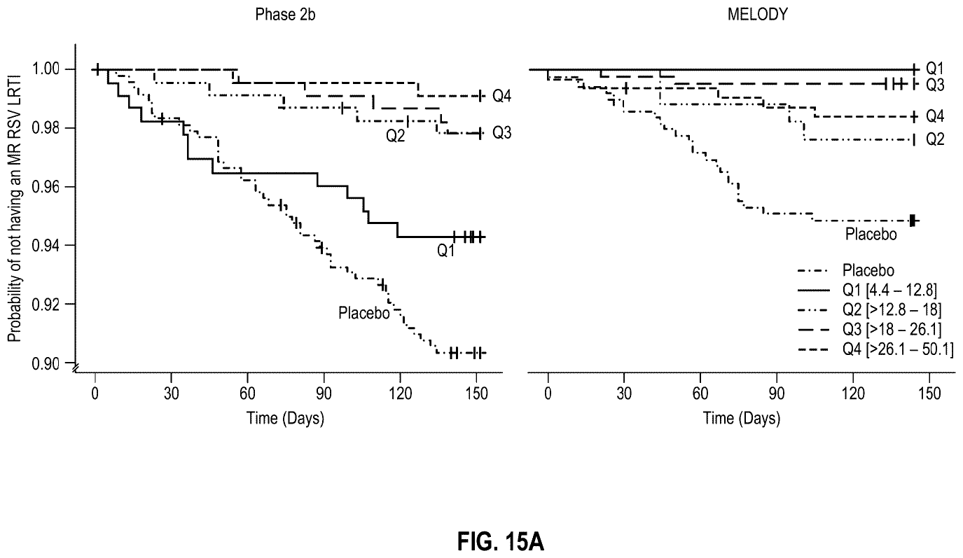

Overall, 860 infants from Phase IIb (median age at randomisation: 1.60 [range 0.1-6.4] months; female: 47.6%) and 1490 infants from MELODY (median age at randomisation: 2.60 [0.03-11.10] months; female: 48.4%) were included. Demographics were comparable across studies, except for gestational age (GA) and age at randomisation. Nirsevimab had an efficacy of 79.5% against MA RSV LRTI, 77.3% against RSV LRTI hospitalisation and 86.0% against very severe RSV LRTI through Day 151 (FIG. 14). Consistent efficacy was observed across the following subgroups: age at randomisation, sex, ancestry, weight, and geographical region, and across endpoints of differing disease severity.

In conclusion, in a pooled analysis of two randomised, placebo-controlled studies, prophylaxis with nirsevimab demonstrated consistent efficacy across severities of RSV LRTI through Day 151.

### Example 5: Population Pharmacokinetics and Exposure-Response of Nirsevimab Against Respiratory Syncytial Virus in Infants, Including Infants at Higher Risk of Severe Disease

In two global, pivotal, placebo-controlled studies, nirsevimab reduced RSV-confirmed medically attended lower respiratory tract infection (LRTI) versus placebo over the RSV season (Phase III NCT03979313: MELODY, healthy term and late preterm infants, 74.5%; Phase IIb NCT02878330): healthy preterm infants, 70.1%). A third randomized, pivotal, palivizumab-controlled study (Phase II/III NCT03959488: MEDLEY) evaluated nirsevimab in infants at higher risk of severe RSV disease, including extremely preterm infants (gestational age <29 weeks), infants with chronic lung disease (CLD) of pre-maturity, and/or congenital heart disease (CHD). Infants in MEDLEY received either one dose of nirsevimab (infants weighing <5 kg, 50 mg; ≥5 kg, 100 mg), then four once-monthly placebo doses, or five once-monthly palivizumab doses (15 mg/kg).

Efficacy in the MEDLEY study was established based on pharmacokinetic extrapolation. Extrapolation relies on the assumptions of similar exposure-response across pediatric populations, justified based on the nirsevimab mechanism of action (binding to RSV preventing viral entry), no endogenous targets, and comparable viral etiology. The analyses described below were performed to support the extrapolation of efficacy from Phase IIb and MELODY to the MEDLEY study.

Nirsevimab was administered as a single IM injection. The MELODY and MEDLEY studies applied weight-band dosing (<5 kg, 50 mg; ≥5 kg, 100 mg); in Phase IIb all infants received 50 mg. Pharmacokinetic data, pooled across studies, were analyzed using a population pharmacokinetic approach. An efficacy exposure target was defined based on exposure-response analysis of the primary endpoint (RSV MALRTI through 150 days post dose), pooled from the Phase IIb and MELODY studies. Individual exposures in MEDLEY subjects were compared versus the exposure target, with the goal of >80% of infants above target to conclude successful extrapolation.

The nirsevimab pharmacokinetic model, including effects of body weight and post-menstrual age, adequately described the data. No difference in pharmacokinetics was found in infants with CLD or CHD. The exposure target for efficacy was determined to be area under the curve (AUC)>12.8 day·mg/mL.

In the overall MEDLEY population 94.3% (558/592) of infants had exposures above the target, and the corresponding numbers in the subgroups of specific interest were: infants with CLD of pre-maturity 94.1% (128/136), infants with CHD 80.3% (53/66), and extreme preterm infants <29 weeks GA without CLD or CHD 93.6% (44/47).

In conclusion, nirsevimab provides protection against RSV disease in infants at higher risk for severe RSV disease.

### Methods for Determining Exposure Target for Efficacy

Exposure-response for medically attended RSV LRTI through 150 days post-dose was assessed based on pooled data from the Phase 2b trial and MELODY (populations as treated with nirsevimab or placebo) to define an exposure target for efficacy. Time-to-first event was evaluated by means of a Cox proportional hazards model, stratified by study and age group at dosing (<3.0 months, ≥3.0-6.0 months, and >6.0 months), with area under the concentration-time curve (AUC; derived from individual estimates of clearance at baseline from the population PK model) as the exposure metric. AUCs were divided into four bins based on quartiles of exposures defined from the Phase 2b trial.

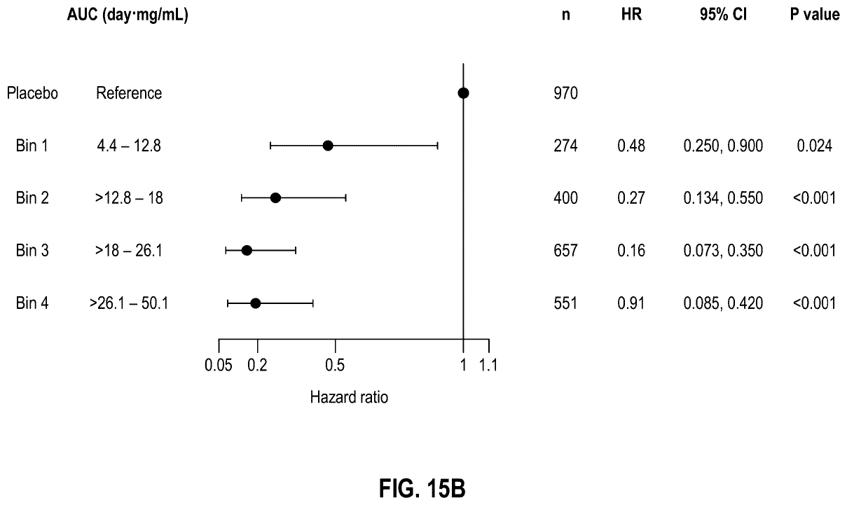

A Kaplan-Meier curve for time to first medically attended RSV LRTI through day 151 by study and exposure bin or placebo is shown in FIG. 15A. The exposure-response analysis showed that an AUC above the first quartile (>12.8 mg·days/mL) provides significant protection from medically attended RSV LRTI versus placebo (p<0.001), with hazard ratios <0.3 (FIGS. 15A and 15B). The hazard ratio for exposures below the first quartile was lower (0.48), supporting AUC >12.8 mg·days/mL as the exposure target (FIG. 15B).

### Example 6: Lack of Temporal Prevalence and Geographic Distribution of Nirsevimab Escape Variants Among Global RSV Strains Since 1956

The nirsevimab binding site has been historically well conserved, but the paucity of recent prospective genomic data limits investigation of temporal evolution and transmission patterns of potential escape variants. This Example reports temporal prevalence, geographic diversity, and resistance profiles of global RSV isolates containing nirsevimab binding site substitutions through 2021.

RSV-positive samples, primarily from infants, were collected and sequenced as part of ongoing INFORM-RSV (global) and OUTSMART-RSV (USA) molecular epidemiology studies (2015-2021). Additional RSV F protein sequences were obtained from NCBI GenBank (1956-2016). Identified RSV F protein substitutions in the nirsevimab binding site (AA 62-69 and AA 196-212) compared to 2013 NLD reference strains were evaluated in a recombinant RSV neutralization susceptibility assay.

Overall, 2,385 published RSV F sequences from 37 countries (RSV A: N=1,525; RSV B: N=860) and 5,675 prospective RSV F sequences from 17 countries (RSV A: N=2,875; RSV B: N=2,800) have been collected and analyzed. More than 98% of amino acids in the nirsevimab binding site have remained highly conserved at all 25 positions in RSV A and at 23 of 25 positions in RSV B during nirsevimab clinical development from 2016 to 2021. In 2015, nirsevimab binding site polymorphisms I206M: Q209R (that maintain susceptibility to nirsevimab neutralization) emerged among circulating RSV B strains. RSV B F variants with reduced susceptibility to nirsevimab neutralization were periodically detected in several different countries at low frequencies (<1.0%), including: L203I (USA, 1993; 3005-fold), K65Q: K68N (KEN, 2012; 1239-fold), K68Q: S211N (NLD and TWN, 2005-2007; 35.7-fold), N201S (ZAF, 2017; 126.7-fold), K68Q: I206M: Q209R (JPN, 2018; 46.4-fold), N201T: 1206M: Q209R (USA, 2018; >417.8-fold), and K68N (CAN, 2019; 29.9-fold). Nirsevimab neutralized all other RSV A and B F protein variants containing binding site substitutions identified in both northern and southern hemispheres.

In conclusion, since 1956, the nirsevimab binding site has remained highly conserved among circulating RSV strains. Nirsevimab escape variants have been rare and have not increased in geotemporal frequency.

### Example 7. Pooled Analysis of Nirsevimab Resistance Through 150 Days Post-Dose in Preterm and Term Infants

In two global, placebo-controlled studies, nirsevimab, a monoclonal antibody to the RSV prefusion (F) protein with extended half-life, reduced medically attended (MA) RSV LRTI versus placebo throughout the RSV season (Phase III: MELODY, healthy term and late preterm infants, 74.5%; Phase IIb: Study 3, healthy preterm infants, 70.1%). This Example summarizes resistance analyses of RT-PCR-confirmed RSV isolates from subjects through 150 days post dose.

Infants were randomized 2:1 to receive one intramuscular injection of nirsevimab or placebo, before their first RSV season. RT-PCR-confirmed RSV isolates were reflexed for genotypic analyses of RSV F and phenotypic analyses of identified substitutions in a recombinant RSV neutralization susceptibility assay.

In the pooled proposed dose analysis of Phase 2b (Study 3; 50 mg nirsevimab if <5 kg at dosing) and MELODY (50 or 100 mg nirsevimab if <5 kg or ≥5 kg at dosing, respectively), no subject with MA RSV LRTI had an RSV isolate containing nirsevimab resistance-associated substitutions in either treatment group (nirsevimab, RSV A: 0/14 and RSV B: 0/5; placebo, RSV A: 0/35 and RSV B: 0/16). In Study 3 (50 mg nirsevimab if ≥5 kg at dosing), 2/18 subjects in the nirsevimab group and 0/20 subjects in the placebo group with MA RSV LRTI had an RSV isolate harbouring nirsevimab binding site substitutions I64T+K68E+1206M+Q209R (>447-fold) or N208S (>387-fold) that conferred reduced susceptibility to nirsevimab neutralization (nirsevimab, RSV A: 0/9 and RSV B: 2/9; placebo, RSV A: 0/10 and RSV B: 0/10). Subjects with RSV isolates harboring F protein sequence variations that maintained susceptibility to nirsevimab neutralization were balanced between treatment groups with no association with RSV disease severity. No subjects with MA RSV LRTI (non-protocol defined) or hospitalization due to any RSV illness had an RSV isolate conferring nirsevimab resistance.

In conclusion, lack of nirsevimab resistance following immunization at the recommended dose supports efficacy and neutralization activity of nirsevimab against both RSV A and B strains throughout the RSV season.

### Example 8. Safety of Nirsevimab for the Prevention of Respiratory Syncytial Virus in Immunocompromised Children: The Phase 2 MUSIC Study

This Example reports the interim safety analysis of the MUSIC trial (NCT04484935), a 12-month Phase 2, open-label, uncontrolled, single-dose study to evaluate the safety and tolerability of nirsevimab, along with the pharmacokinetics and occurrence of antidrug antibodies, in immunocompromised children aged ≤24 months.

Children aged <12 months and entering their first RSV season received a single nirsevimab intramuscular (IM) injection of 50 mg if body weight <5 kg or 100 mg if body weight >5 kg; children aged ≥12 to <24 months and entering their second RSV season received a single nirsevimab IM injection of 200 mg. Subcategories of immunocompromised subjects at the time of informed consent were as follows: A) Primary immunodeficiency; B) Human immunodeficiency virus infection; C) History of organ or bone marrow transplantation; D) Receiving immunosuppressive chemotherapy; E) Receiving systemic high-dose corticosteroid therapy; or F) Receiving other immunosuppressive therapy. Adverse events (AEs), serious AEs (SAEs), AEs of special interest (AESIs; immediate hypersensitivity including anaphylaxis, immune complex disease, or thrombocytopenia), and new onset of chronic disease (NOCDs) were evaluated during 12 months of follow-up. Adverse events were graded by severity according to the National Cancer Institute Common Terminology Criteria for Adverse Events.

A total of 100 immunocompromised children aged ≤24 months were enrolled by 28 Feb. 2022 across Japan (n=26), Ukraine (n=21), USA (n=19), South Africa (n=14), Spain (n=10), Belgium (n=6), Poland (n=3), and the UK (n=1). Of these, a planned interim analysis was performed on 60 children who were enrolled by 31 Dec. 2021 and had follow-up data for ≥151 days post-dose (cut-off date 16 May 2022) or discontinued early. All children had serious, complex underlying medical conditions at baseline: 28 were in immunocompromised subcategory A, 1 was in subcategory B, 12 were in subcategory C, 9 were in subcategory D, 17 were subcategory E, and 9 were subcategory F. One child may have been in multiple immunocompromised subcategories.

All AEs assessed by the investigator to be related to treatment were Grade 1 or 2 in severity. No SAEs or NOCDs were attributed to nirsevimab by the investigator. No anaphylaxis was reported. No serious hypersensitivity reaction was reported. One death (cause unknown) occurred 124 days post-dose; this was determined to be unrelated to treatment by the investigator.

This interim analysis shows that a single IM dose of nirsevimab was well-tolerated and demonstrated a favorable safety profile in immunocompromised children aged ≤24 months. Once-per-RSV-season administration of nirsevimab has the potential to address the significant unmet medical needs to prevent RSV infection in high-risk, immunocompromised children.

### Example 9. Nirsevimab for the Prevention of RSV Disease in Healthy Late-Preterm and Term Infants: Follow-Up Through Second RSV Season

Nirsevimab is a highly neutralizing monoclonal antibody with extended half-life of approximately 70 days. It has been shown to protect term and late preterm infants through their first respiratory syncytial virus (RSV) season against medically attended (MA) RSV lower respiratory tract infections (LRTI) in the Phase 3 MELODY trial (≥35 weeks gestational age, efficacy 74.5%; NCT03979313).

Antibody-dependent enhancement (ADE) could theoretically occur when non-neutralizing antibodies or antibodies in sub-neutralizing concentrations bind to viral antigens without blocking or clearing infection. To address this concern, infants were followed through a second RSV season (510 days post dose) without redosing to evaluate the theoretical risk of ADE in the setting of low nirsevimab concentrations. This Example reports the incidence and disease severity of MA RSV LRTI during the second RSV season.

Infants were randomized 2:1 to receive one intramuscular injection of nirsevimab (infants <5 kg at dosing received 50 mg; infants ≥5 kg received 100 mg) or placebo before their first RSV season. Infants were followed for the detection of cases of MA RSV LRTI for a period of 17 months post-dose. Cases of MA RSV LRTI met predefined clinical criteria of disease severity and were confirmed by real-time reverse-transcriptase polymerase chain reaction. “All cause” referred to any medically attended LRTI or respiratory illness and includes cases of MA RSV LRTI.

Overall, 1490 infants were randomized and included in the intent-to-treat population (994 nirsevimab and 496 placebo) of which 1446 (964 nirsevimab and 482 placebo) were followed through the second season. In the first season, the incidence of MA RSV LRTI was 1.2% in nirsevimab recipients and 5.0% in placebo recipients. In the second season, the incidence was lower, occurring in 0.7% and 0.4% for recipients in the nirsevimab and placebo groups respectively (Table 9). There were no cases of MA RSV LRTI requiring hospitalisation in the second season. Also, the incidence of MA LRTI of any cause and hospitalisation for respiratory illness of any cause was balanced between treatment groups in the second season.

In conclusion, the incidence of MA RSV LRTI in the second RSV season was low and balanced across treatment groups. There was no evidence to support ADE in nirsevimab recipients.

TABLE 9
Incidence of different case definitions of RSV LRTI during two RSV seasons
of the MELODY study (through 511 days post-dose, ITT population)

| | RSV season one: to Day | | | | RSV season two: Days | | | |
|---|---|---|---|---|---|---|---|---|
| | 151 | | | | 361-511 | | | |
| | Placebo | | Nirsevimab | | Placebo | | Nirsevimab | |
| Definition, n (%) | (n = 496) | | (n = 994) | | (n = 482) | | (n = 964) | |
| MA RSV LRTI | 25 | (5.0) | 12 | (1.2) | 2 | (0.4) | 7 | (0.7) |
| MA RSV LRTI with | 8 | (1.6) | 6 | (0.6) | 0 | (0.0) | 0 | (0.0) |
| hospitalization | | | | | | | | |
| All MA LRTI (any cause)* | 78 | (15.7) | 96 | (9.7) | 22 | (4.6) | 37 | (3.8) |
| All MA respiratory illness with | 16 | (3.2) | 24 | (2.4) | 3 | (0.6) | 4 | (0.4) |
| hospitalization (any cause)* | | | | | | | | |

*Any medically attended LRTI or respiratory illness that did not meet the definition applied to MA RSV LRTI ITT = intent-to-treat; LRTI = lower respiratory tract infection; MA = medically attended; RSV = respiratory syncytial virus

### Example 10. The Safety and Efficacy of Nirsevimab on Medically Attended RSV Lower Respiratory Tract Infection in all Infants Enrolled to the Phase 3 MELODY Trial

The Phase 3 MELODY trial (n=1490) evaluated nirsevimab, a single-dose monoclonal antibody with an extended half-life and demonstrated efficacy of 74.5% (95% CI 49.6, 87.1; NCT03979313) against medically attended (MA) respiratory syncytial virus (RSV) lower respiratory tract infection (LRTI; primary endpoint) in late preterm and term infants. This Example summarizes safety and efficacy data of all 3012 infants enrolled in MELODY.

Infants were randomized 2:1 to receive one intramuscular injection of nirsevimab (50 mg if <5 kg at dosing; 100 mg if >5 kg at dosing) or placebo before their first RSV season. Enrolment began on 23 Jul. 2019 and, after pausing due to the COVID-19 pandemic, ended on 22 Oct. 2021. Adverse events (AEs) were monitored for 360 days post-dose in all treated infants. MA RSV LRTI incidence was monitored over 150 days post-dose in all randomized infants. Cases met predefined clinical criteria of disease severity and were laboratory-confirmed. The RSV G gene was sequenced and compared with RSV A or B reference strains for subtyping.

Overall, 2994 infants were dosed (1998 nirsevimab, 996 placebo). Most infants (nirsevimab 93.9%; placebo 93.5%) completed 150 days post-dose follow-up, of which almost half (nirsevimab 48.0%, placebo 48.1%) had completed 360 days at the time of this analysis.

The incidence of AEs was similar across treatment groups (Table 10). Four deaths had occurred at the time of this analysis, in nirsevimab recipients, and were assessed as unrelated to treatment; there were no deaths among placebo recipients.

TABLE 10
Incidence of adverse events through 360 days post-dose (safety population)*

| | Placebo | | Nirsevimab | |
|---|---|---|---|---|
| Event, n (%) | (N = 996) | | (N = 1998) | |
| ≥1 AE | 815 | (81.8) | 1673 | (83.7) |
| ≥1 treatment-related AE | 15 | (1.5) | 25 | (1.3) |
| ≥1 AE ≥ Grade 3 severity | 38 | (3.8) | 61 | (3.1) |
| ≥1 treatment-related AE ≥ Grade 3 severity | 1 | (0.1) | 1 | (0.1) |
| Deaths | 0 | (0.0) | 4 | (0.2)^{†} |
| ≥1 SAE | 74 | (7.4) | 125 | (6.3) |
| ≥1 treatment-related SAE | 1 | (0.1) | 0 | (0.0) |
| ≥1 SAE and/or ≥ Grade 3 severity AE | 76 | (7.6) | 132 | (6.6) |
| ≥1 AESI based on investigator assessment^{‡} | 0 | (0.0) | 4 | (0.2) |
| ≥1 AESI based on selected MedDRA preferred terms^{‡} | 230 | (23.1) | 468 | (23.4) |
| ≥1 treatment-related AESI based on selected MedDRA preferred | 2 | (0.2) | 10 | (0.5) |
| terms^{‡} | | | | |
| ≥1 treatment-related skin reaction | 3 | (0.3) | 12 | (0.6) |
| ≥1 NOCD | 2 | (0.2) | 3 | (0.2) |
| ≥1 treatment-related NOCD | 0 | (0.0) | 0 | (0.0) |
| ≥1 event related to COVID-19 | 44 | (4.4) | 72 | (3.6) |
| ≥1 COVID-19 confirmed event^{§} | 41 | (4.1) | 62 | (3.1) |
| ≥1 COVID-19 suspected event^{¶} | 3 | (0.3) | 10 | (0.5) |

*All patients who were randomized and received any treatment. Approximately 48% of children had completed 360 days post-dose follow-up at the time of this analysis.
^{†}1) Day 139, cause undetermined. Infant had suspected undiagnosed metabolic disease; 2) Day 334, acute gastroenteritis; 3) Day 144, acute gastroenteritis; 4) Day 290, skull base fracture following road traffic accident. All four deaths were assessed by the Investigator as being unrelated to treatment.
^{‡}Comprised hypersensitivity (including anaphylaxis), immune-complex disease, and thrombocytopenia.
^{§}Included positive asymptomatic or symptomatic cases
^{¶}Included those for which signs and symptoms were judged by the investigator to be highly suggestive of Covid 19 but for which results from a confirmatory diagnostic test were unavailable or were negative Severity was graded according to the National Cancer Institute Common Terminology Criteria for Adverse Events, version 5.0 and coded according to the MedDRA version 23.1.
AE, adverse event; AESI, AE of special interest; COVID-19, Coronavirus disease 2019; MedDRA, Medical Dictionary for Regulatory Activities; NOCD, new onset chronic disease; SAE, serious AE

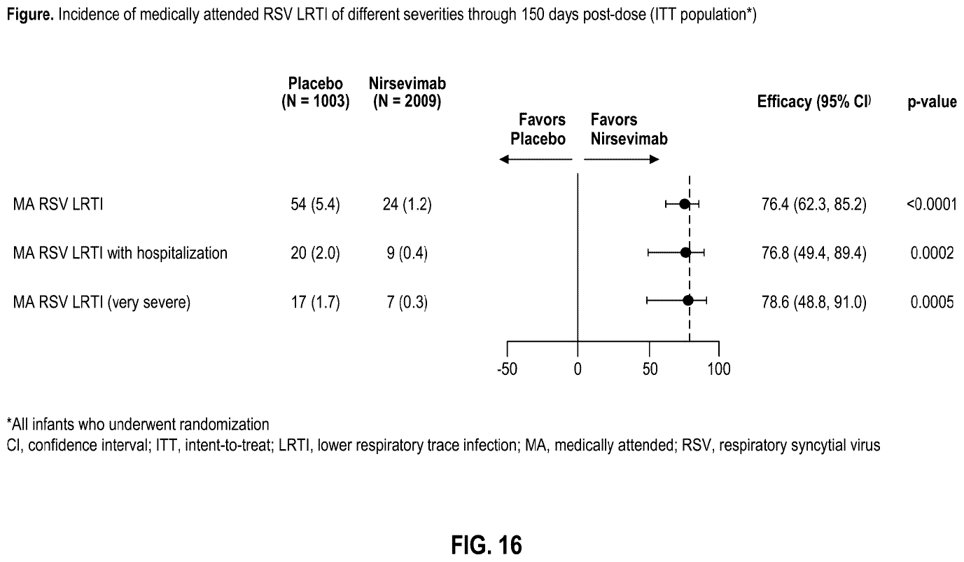

Nirsevimab demonstrated an efficacy of 76.4% (95% CI: 62.3, 85.2) against MA RSV LRTI, 76.8% (95% CI: 49.4, 89.4) against MA RSV LRTI with hospitalization, and 78.6% (95% CI: 48.8, 91.0) against very severe MA RSV LRTI compared with placebo (FIG. 16). A trend to reduction in incidence of RSV subtypes A and B was observed (Table 11).

TABLE 11
Incidence of MA RSV LRTI through 150 days post dose
by RSV subtype and reporting period (ITT population*)

| | Primary reporting period^{†} | | | | Secondary reporting period^{‡} | | | |
|---|---|---|---|---|---|---|---|---|
| | Placebo | | Nirsevimab | | Placebo | | Nirsevimab | |
| n (%) | (n = 496) | | (n = 994) | | (n = 507) | | (n = 1015) | |
| Total RSV | 25 | (5.0) | 12 | (1.2) | 29 | (5.7) | 12 | (1.2) |
| RSV subtype A | 21 | (4.2) | 12 | (1.2) | 4 | (0.8) | 2 | (0.2) |
| RSV subtype B | 4 | (0.8) | 0 | (0.0) | 25 | (4.9) | 10 | (1.0) |

*All infants who underwent randomization
^{†}Northern Hemisphere, 2019/2020; Southern Hemisphere, 2020.
^{‡}Southern Hemisphere, 2021; Northern Hemisphere, 2021/2022.
ITT, intent-to-treat population; LRTI, lower respiratory tract infection; MA, medically attended; RSV, respiratory syncytial virus

Thus, in the complete MELODY study population, a single dose of nirsevimab protected late preterm and term infants against MA RSV LRTI, associated hospitalisations, and severe disease over an entire RSV season.

### Example 11. Antiviral Resistance

This Example describes nirsevimab escape variants as measured in vitro and in vivo.

### In Cell Culture

Escape variants were selected following three passages in cell culture of RSV A2 and B9320 strains in the presence of nirsevimab. Recombinant RSV A variants that showed reduced susceptibility to nirsevimab included those with identified substitutions N671: N208Y (103-fold as compared to reference). Recombinant RSV B variants that showed reduced susceptibility to nirsevimab included those with identified substitutions N208D (>90,000-fold), N208S (>24,000-fold), K68N: N201S (>13,000-fold), or K68N: N208S (>90,000-fold). All resistance-associated substitutions identified among neutralization escape variants were located in the nirsevimab binding site (amino acids 62-69 and 196-212) and were shown to reduce binding affinity to RSV F protein.

### In Surveillance Trials

In prospective, observational, global molecular epidemiology studies (OUTSMART-RSV and INFORM-RSV) genetic diversity of RSV F protein sequences have remained low (most amino acids in both RSV A and RSV B>99% conserved) and prevalence of variants harboring nirsevimab resistance-associated substitutions has been rare (<1%). Since 2015, most amino acid residues in the nirsevimab binding site have remained highly conserved (>99%) at all positions in RSV A and 22 of the 25 positions in RSV B. Co-occurring mutations 1206M: Q209R in the binding site that have become prevalent in RSV B since 2017 retain full susceptibility to nirsevimab (1206M: Q209R, 0.23-fold change). The S211N substitution which has expanded in prevalence also retains susceptibility to nirsevimab, both individually (1.2-fold change) and as co-occurring substitutions (I206M: Q209R: S211N, 0.5-fold change).

### In Clinical Trials

In MELODY, MEDLEY, MUSIC, and subjects who received the recommended dose of 50 mg nirsevimab if <5 kg weight in D5290C00003, no subject with medically attended RSV lower respiratory tract infection (MA RSV LRTI) or any RSV case definition had an RSV isolate containing a consensus nirsevimab resistance associated substitution in any treatment group.

In D5290C00003 (subjects who received a single dose of 50 mg nirsevimab), 2 of 40 subjects with RSV infections corresponding to any case definition had a variant containing nirsevimab resistance associated substitutions. RSV B variants occurred in two subjects receiving below the recommended nirsevimab dose and harboured I64T: K68E: 1206M: Q209R co-occurring substitutions or the N208S substitution that showed reduced susceptibility to nirsevimab (IC50>ULOQ). Resistance associated substitutions were not identified as major variants in any sampling timepoints in MELODY, MEDLEY, or MUSIC studies, including post day 361 when nirsevimab titers have waned.

Minimal data are available that show that variants resistant to nirsevimab could have cross-resistance to other monoclonal antibodies targeting the F protein of RSV; palivizumab retained full neutralisation potency against resistant associated substitutions identified in D5290C00003. Nirsevimab retained activity against recombinant RSV harbouring palivizumab resistance associated substitutions identified in molecular epidemiology studies and in neutralisation escape variants of palivizumab.

### Example 12. Immunogenicity

This Example summarizes measurement of anti-drug antibodies in Phase 2b, MELODY, and MEDLEY trials.

In Phase 2b and MELODY (Primary cohort) anti-nirsevimab antibodies were detected in 84/1498 (5.6%) infants who received a single dose of nirsevimab at the recommended dosing regimen during the 361 days post dosing period, and 68/1423 (4.8%) tested positive for anti-drug antibodies (ADA) against the YTE domain. In MELODY 14/896 (1.6%) of subjects tested positive for nirsevimab neutralising antibodies. For subjects receiving a single dose of nirsevimab in their first RSV season in MEDLEY, anti-nirsevimab antibodies were detected in 32/587 (5.5%) of infants during the 361 days post dosing period. Nirsevimab neutralising antibodies were detected in 2/564 (0.4%) of infants and 31/564 (5.5%) of infants tested positive for ADA against the YTE domain. Of 180 subjects who received a second dose of nirsevimab in their second RSV season, 8 subjects (4.4%) were ADA positive through 360 days post dose of the first RSV season and none had detectable ADA through 150 days of the second RSV season. None of the 10 ADA positive subjects in the second RSV season were positive in the first RSV season. In the second RSV season, 8 subjects had anti-YTE ADA and one of the subjects also had neutralising antibodies. For subjects receiving nirsevimab in their first or second RSV season in MUSIC, anti-nirsevimab antibodies were detected in 0/60 (0.0%) of children during the 151 days post-dosing period. Nirsevimab neutralising antibodies were detected in 0/60 (0.0%) of children and 2/60 (3.3%) of children tested positive for ADA against the YTE domain.

The development of ADA against nirsevimab appears to have no clinically relevant effect on its clearance (up to 5 months), efficacy, or safety.

LIST OF SEQUENCES

| SEQ ID | | |
|---|---|---|
| NO | Description | Sequence |
| 1 | Heavy chain | QVQLVQSGAE VKKPGSSVMV SCQASGGLLE |
| | | 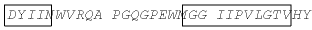 |
| | | 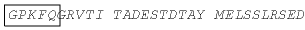 |
| | | 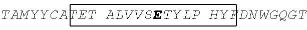 |
| | | LVTVSSASTK GPSVFPLAPS SKSTSGGTAA |
| | | LGCLVKDYFP EPVTVSWNSG ALTSGVHTFP |
| | | AVLQSSGLYS LSSVVTVPSS SLGTQTYICN |
| | | VNHKPSNTKV DKRVEPKSCD KTHTCPPCPA |
| | | PELLGGPSVF LFPPKPKDTL YITREPEVTC |
| | | VVVDVSHEDP EVKENWYVDG VEVHNAKTKP |
| | | REEQYNSTYR VVSVLTVLHQ DWLNGKEYKC |
| | | KVSNKALPAP IEKTISKAKG QPREPQVYTL |
| | | PPSREEMTKN QVSLTCLVKG FYPSDIAVEW |
| | | ESNGQPENNY KTTPPVLDSD GSFFLYSKLT |
| | | VDKSRWQQGN VFSCSVMHEA LHNHYTQKSL SLSPGK |
| 2 | V_{H} | QVQLVQSGAE VKKPGSSVMV SCQASGGLLE |
| | | DYIINWVRQA PGQGPEWMGG IIPVLGTVHY |
| | | GPKFQGRVTI TADESTDTAY MELSSLRSED |
| | | TAMYYCATET ALVVSETYLP HYFDNWGQGT |
| | | LVTVSS |
| 3 | HCDR1 | DYIIN |
| 4 | HCDR2 | GIIPVLGTVHYGPKFQG |
| 5 | HCDR3 | ETALVVSETYLPHYFDN |
| 6 | Light chain | DIQMTQSPSS LSAAVGDRVT ITCQASQDIV |
| | | NYLNWYQQKP GKAPKLLIYV ASNLETGVPS |
| | | RFSGSGSGTD FSLTISSLQP EDVATYYCQQ |
| | | YDNLPLTFGG GTKVEIKRTV AAPSVFIFPP |
| | | SDEQLKSGTA SVVCLLNNFY PREAKVQWKV |
| | | DNALQSGNSQ ESVTEQDSKD STYSLSSTLT |
| | | LSKADYEKHK VYACEVTHQG |
| | | LSSPVTKSEN RGEC |
| 7 | V_{L} | DIQMTQSPSS LSAAVGDRVT ITCQASQDIV |
| | | NYLNWYQQKP GKAPKLLIYV ASNLETGVPS |
| | | RFSGSGSGTD FSLTISSLQP EDVATYYCQQ |
| | | YDNLPLTFGG GTKVEIK |
| 8 | LCDR1 | QASQDIVNYLN |
| 9 | LCDR2 | VASNLET |
| 10 | LCDR3 | QQYDNLPLT |
| 11 | Heavy chain | QVQLVQSGAE VKKPGSSVMV SCQASGGLLE |
| | without C- | DYIINWVRQA PGQGPEWMGG IIPVLGTVHY |
| | terminal | GPKFQGRVTI TADESTDTAY MELSSLRSED |
| | lysine | TAMYYCATET ALVVSETYLP HYFDNWGQGT |
| | | LVTVSSASTK GPSVFPLAPS SKSTSGGTAA |
| | | LGCLVKDYFP EPVTVSWNSG ALTSGVHTFP |
| | | AVLQSSGLYS LSSVVTVPSS SLGTQTYICN |
| | | VNHKPSNTKV DKRVEPKSCD KTHTCPPCPA |
| | | PELLGGPSVF LFPPKPKDTL YITREPEVTC |
| | | VVVDVSHEDP EVKENWYVDG VEVHNAKTKP |
| | | REEQYNSTYR VVSVLIVLHQ DWLNGKEYKC |
| | | KVSNKALPAP IEKTISKAKG QPREPQVYTL |
| | | PPSREEMTKN QVSLTCLVKG FYPSDIAVEW |
| | | ESNGQPENNY KTTPPVLDSD GSFFLYSKLT |
| | | VDKSRWQQGN VFSCSVMHEA LHNHYTQKSL SLSPG |

1. A method of preventing respiratory syncytial virus (RSV) lower respiratory tract disease (LRTD) in a subject from birth, comprising administering to the subject a single dose of nirsevimab, wherein the single dose is effective to prevent RSV LRTD for more than five months.

2. The method of claim 1, wherein the subject is administered the single dose of nirsevimab about two weeks, about three weeks, about four weeks, about one month, about two months, or about three months before the beginning of the RSV season.

3. The method of claim 2, wherein the beginning of the RSV season is defined by the first two consecutive weeks during which the average percentage of positive RSV tests over the two-week period exceeds a threshold, wherein the threshold is from 3%-13%, optionally wherein (i) the RSV tests are PCR tests and the threshold is 3% or (ii) the RSV tests are antigen tests and the threshold is 10%.

4. The method of claim 1, wherein the subject is administered the single dose of nirsevimab following the end of the last RSV season, wherein the single dose is effective to prevent RSV LRTD throughout the next RSV season.

5. The method of claim 4, wherein the subject is administered the single dose of nirsevimab about one week, about two weeks, about three weeks, about four weeks, about one month, about two months, about three months, about four months, about five months, about six months, or about seven months following the end of the last RSV season.

6. The method of claim 4, wherein the end of the last RSV season is defined by the first week during which the average percentage of positive RSV tests over the one-week period is below a threshold, wherein the threshold is from 3%-13%, optionally wherein (i) the RSV tests are PCR tests and the threshold is 3% or (ii) the RSV tests are antigen tests and the threshold is 10%.

7. The method of claim 1, wherein the subject is at a high risk of developing an RSV infection.

8. The method of claim 7, wherein the subject is born with a gestational age less than 29 weeks; and/or has chronic lung disease (CLD), congenital heart disease (CHD), a suppressed immune system, a compromised immune system, an immunodeficiency, a neuromuscular disorder, Down's syndrome, a congenital airway anomaly, and/or cystic fibrosis.

9. The method of claim 1, wherein the amount of nirsevimab in the single dose is effective to yield serum AUC_{0-∞} of greater than 12.8 day·mg/mL in the subject.

10. The method of claim 1, wherein
a) the amount of nirsevimab in the single dose is effective to yield elevated RSV neutralizing antibody (Nab) levels in the subject at about 12 months after administration compared to the subject's RSV Nab levels at the time of administration; and/or
b) the amount of nirsevimab in the single dose is effective to yield elevated RSV Nab levels in the subject at about 12 months after administration compared to RSV Nab levels of an individual with a confirmed RSV infection and who was not administered nirsevimab.

11. The method of claim 1, wherein the amount of nirsevimab in the single dose is
50 mg if the subject weighs <5 kg at the time of administration, or
100 mg if the subject weight ≥5 kg at the time of administration.

12. The method of claim 1, wherein preventing RSV LRTD comprises preventing
a) medically attended RSV LRTD;
b) RSV-associated hospitalization;
c) severe RSV infection; and/or
d) very severe RSV infection.

13. (canceled)

14. The method of claim 1, wherein the method further prevents all-cause lower respiratory tract infection (LRTI) or all-cause LRTI hospitalization in the subject.

15. The method of claim 1, wherein the amount of nirsevimab in the single dose is effective to reduce the subject's antibiotic use compared to the antibiotic use of an individual who was not administered nirsevimab.

16. The method of claim 1, wherein the single dose of nirsevimab is administered to the subject intramuscularly or subcutaneously.

17. A method of preventing respiratory syncytial virus (RSV) lower respiratory tract disease (LRTD) in a subject at high risk of developing an RSV infection, comprising administering to the subject a first dose of nirsevimab before the subject's first RSV season and a second dose of nirsevimab before the subject's second RSV season, wherein the amount of nirsevimab in the first dose is
50 mg if the subject weighs <5 kg at the time of administration, and
100 mg if the subject weighs ≥5 kg at the time of administration;
and wherein the amount of nirsevimab in the second dose is 200 mg, wherein the first dose of nirsevimab is effective to prevent RSV LRTD in the subject for more than five months.

18. A method of preventing respiratory syncytial virus (RSV) lower respiratory tract disease (LRTD) in a subject undergoing a heart surgery, wherein the method comprises:
A)
a) administering to the subject a first dose of nirsevimab before or during the subject's first RSV season, wherein
i) the amount of nirsevimab in the first dose is 50 mg if the subject weighs <5 kg at the time of administration of the first dose; and
ii) the amount of nirsevimab in the first dose is 100 mg if the subject weighs >5 kg at the time of administration of the first dose; and
b) administering to the subject a second dose of nirsevimab after the heart surgery, wherein
i) the amount of nirsevimab in the second dose is 50 mg if the subject weighs <5 kg at the time of administration of the second dose and the second dose is administered within 90 days of administration of the first dose;
ii) the amount of nirsevimab in the second dose is 100 mg if the subject weighs >5 kg at the time of administration of the second dose and the second dose is administered within 90 days of administration of the first dose; and
iii) the amount of nirsevimab in the second dose is 50 mg if the second dose is administered more than 90 days after administration of the first dose, or
B)
a) administering to the subject a first dose of nirsevimab after the subject's first season and before or during the subject's second RSV season, wherein the amount of nirsevimab in the first dose is 200 mg; and
b) administering to the subject a second dose of nirsevimab after the heart surgery, wherein
i) the amount of nirsevimab in the second dose is 200 mg if the second dose is administered within 90 days of administration of the first dose; and
ii) the amount of nirsevimab in the second dose is 100 mg if the second dose is administered more than 90 days after administration of the first dose,
optionally wherein the second dose is administered as soon as the subject is stable after the surgery; further optionally wherein the heart surgery is a cardiac surgery with cardiopulmonary bypass.

19. The method of claim 1, wherein the single dose of nirsevimab is administered to the subject at or shortly after birth.

20.-22. (canceled)

23. The method of claim 1, wherein the single dose of nirsevimab is effective to prevent RSV LRTD in the subject for at least eight months.

24. The method of claim 1, wherein the single dose of nirsevimab is effective to prevent RSV LRTD in the subject for at least twelve months.


SEQUENCE LISTING (11 sequences)

SEQ ID NO: 1 - protein, 456 aa, synthetic construct
QVQLVQSGAEVKKPGSSVMVSCQASGGLLEDYIINWVRQAPGQGPEWMGGIIPVLGTVHYGPKFQGRVTITADESTDTAYMELSSLRSEDTAMYYCATETALVVSETYLPHYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 126 aa, synthetic construct [sequence omitted]

SEQ ID NO: 3 - protein, 5 aa, synthetic construct
DYIIN

SEQ ID NO: 4 - protein, 17 aa, synthetic construct
GIIPVLGTVHYGPKFQG

SEQ ID NO: 5 - protein, 17 aa, synthetic construct
ETALVVSETYLPHYFDN

SEQ ID NO: 6 - protein, 214 aa, synthetic construct
DIQMTQSPSSLSAAVGDRVTITCQASQDIVNYLNWYQQKPGKAPKLLIYVASNLETGVPSRFSGSGSGTDFSLTISSLQPEDVATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, synthetic construct [sequence omitted]

SEQ ID NO: 8 - protein, 11 aa, synthetic construct
QASQDIVNYLN

SEQ ID NO: 9 - protein, 7 aa, synthetic construct
VASNLET

SEQ ID NO: 10 - protein, 9 aa, synthetic construct
QQYDNLPLT

SEQ ID NO: 11 - protein, 455 aa, synthetic construct
QVQLVQSGAEVKKPGSSVMVSCQASGGLLEDYIINWVRQAPGQGPEWMGGIIPVLGTVHYGPKFQGRVTITADESTDTAYMELSSLRSEDTAMYYCATETALVVSETYLPHYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG